SEQUENCE LISTING (116 sequences)

SEQ ID NO: 1 - protein, 575 aa, Homo sapiens
MSAATHSPMMQVASGNGDRDPLPPGWEIKIDPQTGWPFFVDHNSRTTTWNDPRVPSEGPKETPSSANGPSREGSRLPPAREGHPVYPQLRPGYIPIPVLHEGAENRQVHPFHVYPQPGMQRFRTEAAAAAPQRSQSPLRGMPETTQPDKQCGQVAAAAAAQPPASHGPERSQSPAASDCSSSSSSASLPSSGRSSLGSHQLPRGYISIPVIHEQNVTRPAAQPSFHQAQKTHYPAQQGEYQTHQPVYHKIQGDDWEPRPLRAASPFRSSVQGASSREGSPARSSTPLHSPSPIRVHTVVDRPQQPMTHRETAPVSQPENKPESKPGPVGPELPPGHIPIQVIRKEVDSKPVSQKPPPPSEKVEVKVPPAPVPCPPPSPGPSAVPSSPKSVATEERAAPSTAPAEATPPKPGEAEAPPKHPGVLKVEAILEKVQGLEQAVDNFEGKKTDKKYLMIEEYLTKELLALDSVDPEGRADVRQARRDGVRKVQTILEKLEQKAIDVPGQVQVYELQPSNLEADQPLQAIMEMGAVAADKGKKNAGNAEDPHTETQQPEATAAATSNPSSMTDTPGNPAAP

SEQ ID NO: 2 - DNA, 1728 bp, Homo sapiens
  source 1..1728: mol_type = genomic DNA
atgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtag

SEQ ID NO: 3-10 - skipped (8 entries)

SEQ ID NO: 11 - DNA, 10 bp, unidentified
  source 1..10: mol_type = genomic DNA
  misc_feature 1..10: note = kozak sequence
gccaccatgg

SEQ ID NO: 12 - skipped

SEQ ID NO: 13 - RNA, 13 nt, unidentified
  source 1..13: mol_type = genomic RNA
  misc_feature 1..13: note = kozak sequence
gccgccrccatgg

SEQ ID NO: 14 - RNA, 10 nt, unidentified
  source 1..10: mol_type = genomic RNA
  misc_feature 1..10: note = kozak sequence
gacaccatgg

SEQ ID NO: 15 - DNA, 141 bp, Adeno-associated virus
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 16 - DNA, 168 bp, Adeno-associated virus
  source 1..168: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 17 - DNA, 170 bp, Adeno-associated virus
  source 1..170: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 18 - DNA, 145 bp, Adeno-associated virus
  source 1..145: mol_type = genomic DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 19 - DNA, 141 bp, Adeno-associated virus
  source 1..141: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 168 bp, Adeno-associated virus
  source 1..168: mol_type = genomic DNA
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 21 - DNA, 145 bp, Adeno-associated virus
  source 1..145: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 22 - DNA, 124 bp, Synthetic construct
  source 1..124: mol_type = other DNA
  misc_feature 1..124: note = vector filler sequence
gcggcaattcagtcgataactataacggtcctaaggtagcgatttaaatacgcgctctcttaaggtagccccgggacgcgtcaattgactacaaaccgagtatctgcagagggccctgcgtatg

SEQ ID NO: 23 - DNA, 84 bp, synthetic construct
  source 1..84: mol_type = other DNA
  misc_feature 1..84: note = vector filler sequence
cttctgaggcggaaagaaccagatcctctcttaaggtagcatcgagatttaaattagggataacagggtaatggcgcgggccgc

SEQ ID NO: 24 - DNA, 63 bp, synthetic construct
  source 1..63: mol_type = other DNA
  misc_feature 1..63: note = vector filler sequence
gttacccaggctggagtgcagtggcacatttctgctcactgcaacctcctcctccctgggttc

SEQ ID NO: 25 - DNA, 573 bp, synthetic construct
  misc_feature 1..573: note = CAG promoter in part Human betaherpesvirus 5
  source 1..573: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 26-30 - skipped (5 entries)

SEQ ID NO: 31 - DNA, 771 bp, synthetic construct
  source 1..771: mol_type = other DNA
acccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 32 - DNA, 544 bp, Homo sapiens
  source 1..544: mol_type = genomic DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcag

SEQ ID NO: 33 - DNA, 502 bp, Homo sapiens
  source 1..502: mol_type = genomic DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagt

SEQ ID NO: 34 - DNA, 5464 bp, Mus musculus
  source 1..5464: mol_type = genomic DNA
ggtaccggatcctgcaaggtcacacaagggtctccacccaccaggtgccctagtctcaatttcagtttccatgccttgttctcacaatgctggcctccccagagctaatttggactttgtttttatttcaaaagggcctgaatgaggagtagatcttgtgctacccagctctaagggtgcccgtgaagccctcagacctggagcctttgcaacagccctttaggtggaagcagaataaagcaattttccttaaagccaaaatcctgcctctagactcttcttctctgacctcggtccctgggctctagggtggggaggtggggcttggaagaagaaggtggggaagtggcaaaagccgatccctagggccctgtgaagttcggagccttccctgtacagcactggctcatagatcctcctccagccaaacatagcaagaagtgatacctcctttgtgacttccccaggcccagtacctgtcaggttgaaacaggatttagagaagcctctgaactcacctgaactctgaagctcatccaccaagcaagcacctaggtgccactgctagttagtatcctacgctgataatatgcagagctgggccacagaagtcctggggtgtaggaactgaccagtgacttttcagtcggcaaaggtatgaccccctcagcagatgtagtaatgtccccttagatcccatcccaggcaggtctctaagaggacatgggatgagagatgtagtcatgtggcattccaaacacagctatccacagtgtcccttgccccttccacttagccaggaggacagtaaccttagcctatctttcttcctccccatcctcccaggacacaccccctggtctgcagtattcatttcttccttcacgtcccctctgtgacttccatttgcaaggcttttgacctctgcagctgctggaagatagagtttggccctaggtgtggcaagccatctcaagagaaagcagacaacagggggaccagattttggaaggatcaggaactaaatcactggcgggcctgggggtagaaaaaagagtgagtgagtccgctccagctaagccaagctagtccccgagatactctgccacagctgggctgctcggggtagctttaggaatgtgggtctgaaagacaatgggattggaagacatctctttgagtctcccctcaaccccacctacagacacactcgtgtgtggccagactcctgttcaacagccctctgtgttctgaccactgagctaggcaaccagagcatgggccctgtgctgaggatgaagagttggttaccaatagcaaaaacagcaggggagggagaacagagaacgaaataaggaaggaagaaggaaaggccagtcaatcagatgcagtcagaagagatgggaagccaacacacagcttgagcagaggaaacagaaaagggagagattctgggcataaggaggccacagaaagaagagcccaggccccccaagtctcctctttataccctcatcccgtctcccaattaagcccactcttcttcctagatcagacctgagctgcagcgaagagacccgtagggaggatcacactggatgaaggagatgtgtggagaagtccagggaacctaagagccagagcctaaaagagcaagagataaaggtgcttcaaaggtggccaggctgtgcacacagagggtcgaggactggtggtagagcctcaagataaggatgatgctcagaatgggcggggggggggattctgggggggggagagagaaggtgagaaggagcctggaacagagaatctggaagcgctggaaacgataccataaagggaagaacccaggctacctttagatgtaaatcatgaaagacagggagaagggaagctggagagagtagaaggaccccggggcaagacattgaagcaaggacaagccaggttgagcgctccgtgaaatcagcctgctgaaggcagagccctggtatgagcaccagaacagcagaggctagggttaatgtcgagacagggaacagaaggtagacacaggaacagacagagacgggggagccaggtaacaaaggaatggtccttctcacctgtggccagagcgtccatctgtgtccacatactctagaatgttcatcagactgcagggctggcttgggaggcagctggaaagagtatgtgagagccaggggagacaagggggcctaggaaaggaagaagagggcaaaccaggccacacaagagggcagagcccagaactgagttaactccttccttgttgcatcttccataggaggcagtgggaactctgtgaccaccatcccccatgagcccccactacccataccaagtttggcctgagtggcattctaggttccctgaggacagagcctggcctttgtctcttggacctgacccaagctgacccaatgttctcagtaccttatcatgccctcaagagcttgagaaccaggcagtgacatattaggccatgggctaaccctggagcttgcacacaggagcctcaagtgacctccagggacacagctgcagacaggtggcctttatccccaaagagcaaccatttggcataggtggctgcaaatgggaatgcaaggttgaatcaggtcccttcaagaatactgcatgcaagacctaagacccctggagagaggggtatgctcctgcccccacccaccataaggggagtgaactatcctagggggctggcgaccttggggagacaccacattactgagagtgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgtctcctgaacacaatgtctacttagttataacaggcatgacctgctaaagacccaacatctacgacctctgaaaagacagcagccctggaggacaggggttgtctctgagccttgggtgcttgatggtgccacaaaggagggcatgagtgtgagtataaggccccaggagcgttagagaagggcacttgggaaggggtcagtctgcagagcccctatccatggaatctggagcctggggccaactggtgtaaatctctgggcctgccaggcattcaaagcagcacctgcatcctctggcagcctggggaggcggaagggagcaaccccccacttataccctttctccctcagccccaggattaacacctctggccttcccccttcccacctcccatcaggagtggagggttgcagagggagggtaaaaacctacatgtccaaacatcatggtgcacgatatatggatcagtatgtgtagaggcaagaaaggaaatctgcaggcttaactgggttaatgtgtaaagtctgtgtgcatgtgtgtgtgtctgactgaaaacgggcatggctgtgcagctgttcagttctgtgcgtgaggttaccagactgcaggtttgtgtgtaaattgcccaaggcaaagtgggtgaatcccttccatggtttaaagagattggatgatggcctgcatctcaaggaccatggaaaatagaatggacactctatatgtgtctctaagctaaggtagcaaggtctttggaggacacctgtctagagatgtgggcaacagagactacagacagtatctgtacagagtaaggagagagaggagggggtgtagaattctcttactatcaaagggaaactgagtcgtgcacctgcaaagtggatgctctccctagacatcatgactttgtctctggggagccagcactgtggaacttcaggtctgagagagtaggaggctcccctcagcctgaagctatgcagatagccagggttgaaagggggaagggagagcctgggatgggagcttgtgtgttggaggcaggggacagatattaagcctggaagagaaggtgacccttacccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtcctcctgtcacctccagagccaagggatcaaaggaggaggagccaggacaggagggaagtgggagggagggtcccagcagaggactccaaatttaggcagcaggcatatgggatgggatataaaggggctggagcactgagagctgtcagagatttctccaacccaggtaagagggagtttcgggtgggggctcttcacccacaccagacctctccccacctagaaggaaactgcctttcctggaagtggggttcaggccggtcagagatctgacagggtggccttccaccagcctgggaagttctcagtggcaggaggtttccacaagaaacactggatgccccttcccttacgctgtcttctccatcttcctcctggggatgctcctccccgtcttggtttatcttggctcttcgtcttcagcaagatttgccctgtgctgtccactccatctttctctactgtctccgtgccttgccttgccttcttgcgtgtccttcctttccacccatttctcacttcaccttttctccccttctcatttgtattcatccttccttccttccttccttccttccttccttccttccttccttcctttctcccttccttccttccttccttccttccttccttccttccttcctgtgtcagagtgctgagaatcacacctggggttcccacccttatgtaaacaatcttccagtgagccacagcttcagtgctgctgggtgctctcttaccttcctcaccccctggcttgtcctgttccatcctggtcaggatctctagattggtctcccagcctctgctactcctcttcctgcctgttcctctctctgtccagctgcgccactgtggtgcctcgttccagctgtggtccacattcttcaggattctctgaaaagttaaccaggtgagaatgtttcccctgtagacagcagatcacgattctcccggaagtcaggcttccagccctctctttctctgcccagctgcccggcactcttagcaaacctcaggcacccttaccccacatagacctctgacagagaagcaggcactttacatggagtcctggtgggagagccataggctacggtgtaaaagaggcagggaagtggtggtgtaggaaagtcaggacttcacatagaagcctagcccacaccagaaatgacagacagatccctcctatctcccccataagagtttgagtcgacccgcggccccgaattg

SEQ ID NO: 35 - DNA, 413 bp, Gallus gallus
  source 1..413: mol_type = genomic DNA
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 36 - DNA, 1090 bp, Homo sapiens
  source 1..1090: mol_type = genomic DNA
ctctcagccctggaagtccttgctcacagccgaggcgccgagagcgcttgctctgcccagatctgcgcgagtctggcgcccgcgctctgaacggcgtcgctgcccagcccccttccccgggaggtgggagcggccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttcctttgggtcagtgtcacctccaggatacagaca

SEQ ID NO: 37 - DNA, 253 bp, Homo sapiens
  source 1..253: mol_type = genomic DNA
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 38 - DNA, 281 bp, Gallus gallus
  source 1..281: mol_type = genomic DNA
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 39 - DNA, 220 bp, Human betaherpesvirus 5
  source 1..220: mol_type = genomic DNA
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 40 - DNA, 583 bp, Human betaherpesvirus 5
  source 1..583: mol_type = genomic DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 41 - DNA, 508 bp, Human betaherpesvirus 5
  source 1..508: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 42 - DNA, 573 bp, synthetic construct; identical to SEQ ID NO: 25
  misc_feature 1..573: note = CAG promoter in part Human betaherpesvirus 5
  source 1..573: mol_type = other DNA

SEQ ID NO: 43 - DNA, 580 bp, synthetic construct
  misc_feature 1..580: note = CAG promoter in part Human betaherpesvirus 5
  source 1..580: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 44 - DNA, 455 bp, Homo sapiens
  source 1..455: mol_type = genomic DNA
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 45-47 - skipped (3 entries)

SEQ ID NO: 48 - DNA, 281 bp, Homo sapiens
  source 1..281: mol_type = genomic DNA
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 49 - skipped

SEQ ID NO: 50 - DNA, 293 bp, Human betaherpesvirus 5
  source 1..293: mol_type = genomic DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 51 - DNA, 953 bp, Homo sapiens
  source 1..953: mol_type = genomic DNA
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 52 - DNA, 1068 bp, synthetic construct
  misc_feature 1..1068: note = Chicken beta-actin exon/intron plus rabbit globin intron
  source 1..1068: mol_type = other DNA
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 53 - DNA, 149 bp, synthetic construct
  source 1..149: mol_type = other DNA
ggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 54 - DNA, 126 bp, Homo sapiens
  source 1..126: mol_type = genomic DNA
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 55 - DNA, 121 bp, Human betaherpesvirus 5
  source 1..121: mol_type = genomic DNA
tcagatcgcctggagaggccatccacgctgttttgacctccatagtggacaccgggaccgatccagcctccgcggccgggaacggtgcattggaacgcggattccccgtgccaagagtgac

SEQ ID NO: 56 - DNA, 512 bp, synthetic construct
  misc_feature 1..512: note = adenovirus derived enhancer element
  source 1..512: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 57 - DNA, 956 bp, Homo sapiens
  source 1..956: mol_type = genomic DNA
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 58 - DNA, 939 bp, Homo sapiens
  source 1..939: mol_type = genomic DNA
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 59 - DNA, 83 bp, Homo sapiens
  source 1..83: mol_type = genomic DNA
tcagaagccccgggctcgtcagtcaaaccggttctctgtttgcactcggcagcacgggcaggcaagtggtccctaggttcggg

SEQ ID NO: 60 - DNA, 476 bp, Homo sapiens
  source 1..476: mol_type = genomic DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 61 - DNA, 196 bp, simian virus 40
  source 1..196: mol_type = genomic DNA
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 62 - DNA, 589 bp, synthetic construct
  misc_feature 1..589: note = mutated woodchuck hepatitis regulatory element
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 63 - DNA, 588 bp, synthetic construct
  misc_feature 1..588: note = mutated woodchuck hepatitis regulatory element
  source 1..588: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 64 - DNA, 755 bp, synthetic construct
  misc_feature 1..755: note = mutated woodchuck hepatitis regulatory element
  source 1..755: mol_type = other DNA
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 65 - DNA, 12 bp, synthetic construct
  misc_feature 1..12: note = CAAX motif
  source 1..12: mol_type = other DNA
tgtgtgataatg

SEQ ID NO: 66 - DNA, 810 bp, Homo sapiens
  source 1..810: mol_type = genomic DNA
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 67 - DNA, 726 bp, Hepatitis B virus
  source 1..726: mol_type = genomic DNA
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 68 - DNA, 755 bp, synthetic construct
  misc_feature 1..755: note = HepB derived enhancer element
  source 1..755: mol_type = other DNA
ttcctgtaaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagctggactggagcaaacctcatcgggaccgacaattctgtcgtactctcccgcaagcactcaccgtttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 69 - DNA, 94 bp, Homo sapiens
  source 1..94: mol_type = genomic DNA
gctggagcctcggtagccgttcctcctgcccgctgggcctcccaacgggccctcctcccctccttgcaccggcccttcctggtctttgaataaa

SEQ ID NO: 70 - DNA, 596 bp, Woodchuck hepatitis virus
  source 1..596: mol_type = genomic DNA
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 71 - DNA, 387 bp, Oryctolagus cuniculus
  source 1..387: mol_type = genomic DNA
tggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 72 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaatacaatagcaggcatgctggggatgcggtgggctctatgggtacccaggtgctgaagaattgacccggttcctcctggg

SEQ ID NO: 73 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctggg

SEQ ID NO: 74 - DNA, 225 bp, Bos taurus
  source 1..225: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 75 - DNA, 202 bp, Homo sapiens
  source 1..202: mol_type = genomic DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 76-95 - skipped (20 entries)

SEQ ID NO: 96 - protein, 735 aa, Adeno-associated virus 2 [sequence omitted]

SEQ ID NO: 97 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 98 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 99 - protein, 738 aa, Adeno-associated virus
  REGION 1..738: note = Non-human primate AAV serotype rh10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 100 - DNA, 2217 bp, Adeno-associated virus
  source 1..2217: mol_type = genomic DNA
  misc_feature 1..2217: note = non-human primate AAV serotype rh74
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 101 - protein, 738 aa, Adeno-associated virus
  REGION 1..738: note = Non-human primate AAV serotype rh74 VP1
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 102 - protein, 601 aa, Adeno-associated virus
  REGION 1..601: note = Non-human primate AAV serotype rh74 VP2
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 103 - protein, 535 aa, Adeno-associated virus
  REGION 1..535: note = Non-human primate AAV serotype rh74 VP3
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 104 - protein, 743 aa, Adeno-associated virus
  REGION 1..743: note = AAV variant PHP. B
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 105 - protein, 7 aa, synthetic construct
  REGION 1..7: note = Peptide insert
TLAVPFK

SEQ ID NO: 106 - protein, 7 aa, synthetic construct
  REGION 1..7: note = Peptide insert
KFPVALT

SEQ ID NO: 107 - DNA, 3754 bp, synthetic construct
  source 1..3754: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtagtcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 108 - DNA, 3378 bp, synthetic construct
  source 1..3378: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtagtcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 109 - DNA, 3305 bp, synthetic construct
  source 1..3305: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtcctgcagggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaacccgccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtagtcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 110 - DNA, 4537 bp, synthetic construct
  source 1..4537: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 111 - DNA, 4161 bp, synthetic construct
  source 1..4161: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 112 - DNA, 4088 bp, synthetic construct
  source 1..4088: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtcctgcagggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaacccgccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 113 - DNA, 5260 bp, synthetic construct
  source 1..5260: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgggatccggcagtggagagggcagaggaagtctgctaacatgcggtgacgtcgaggagaatcctggcccaatgagcaagggcgaggagctgttcaccggcgtggtgcccatcctggtggagctggacggcgacgtgaacggccacaagttcagcgtgagaggcgagggcgagggcgacgccaccaacggcaagctgaccctgaagttcatctgcaccaccggcaagctgcccgtgccctggcccaccctggtgaccaccctgacctacggcgtgctgtgcttcagcagataccccgaccacatgaagagacacgacttcttcaagagcgccatgcccgagggctacgtgcaggagagaaccatcagcttcaaggacgacggcacctacaagaccagagccgaggtgaagttcgagggcgacaccctggtgaacagaatcgagctgaagggcatcgacttcaaggaggacggcaacatcctgggccacaagctggagtacaacttcaacagccacaacgtgtacatcaccgccgacaagcagaagaacggcatcaaggcctacttcaagatcagacacaacgtggaggacggcagcgtgcagctggccgaccactaccagcagaacacccccatcggcgacggccccgtgctgctgcccgacaaccactacctgagcacccagagcgtgctgagcaaggaccccaacgagaagagagaccacatggtgctgctggaggacgtgaccgccgccggcatcacccacggcatggacgagctgtacaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 114 - DNA, 4053 bp, synthetic construct
  source 1..4053: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcctgaggacgcgtgatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaaggccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtagtcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 115 - DNA, 471 bp, Homo sapiens
  source 1..471: mol_type = genomic DNA
cctgcagggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 116 - DNA, 1219 bp, Homo sapiens
  source 1..1219: mol_type = genomic DNA
gatctggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaag

(19) United States
(12) Patent Application Publication
(10) Pub. No.: US 2025/0269065 A1
(43) Pub. Date: Aug. 28, 2025

(54) B-CELL LYMPHOMA 2-ASSOCIATED ANTHANOGENE 3 (BAG3) GENE THERAPY USING AAV VECTOR

(71) Applicant: SPACECRAFT SEVEN, LLC, Cranbury, NJ (US)

(72) Inventors: Christopher Dean HERZOG, Cranbury, NJ (US); Chester Bittencort SACRAMENTO, Cranbury, NJ (US); David RICKS, Cranbury, NJ (US); Raj PRABHAKAR, Cranbury, NJ (US)

(73) Assignee: SPACECRAFT SEVEN, LLC, Cranbury, NJ (US)

(21) Appl. No.: 18/858,272

(22) PCT Filed: Apr. 21, 2023
(86) PCT No.: PCT/US2023/066054

§ 371 Date: Oct. 18, 2024

Related U.S. Application Data
(60) Provisional application No. 63/333,798, filed on Apr. 22, 2022.

(51) Int. Cl. A61K 48/00; A61K 38/17; A61P 9/04; C12N 15/86

(52) U.S. Cl. CPC A61K 48/0058; A61K 38/1709; A61K 48/0075; A61K 48/0083; A61P 9/04; C12N 15/86; C12N 2750/14143; C12N 2830/008; C12N 2830/48; C12N 2830/50

(57) ABSTRACT

Provided herein is a gene therapy for BAG3 (B-cell Lymphoma 2-Associated Anthanogene 3), e.g., using an adeno-associated virus (AAV) vector. The promoter of the vector may be a MHCK7 promoter, a cardiac troponin T (hTNNT2) promoter, a heat shock protein 70 (HSP70) promoter, or a ubiquitin C (UBC) promoter. The capsid may be an AAVrh.74 or AAV9 capsid or a functional variant thereof. In certain embodiments, the capsid is an AAVrh.74 capsid or functional variant thereof. Other promoters or capsids may be used. Further provided are methods of treatment, such as by intravenous, intracoronary, intracarotid or intracardiac administration of the AAV vector, and other compositions and methods.

### CROSS-REFERENCE TO RELATED APPLICATIONS

This application claims benefit of priority to U.S. Provisional Patent Application No. 63/333,798, filed Apr. 22, 2022, the disclosure of which is incorporated herein by reference in its entirety for all purposes.

### STATEMENT REGARDING THE SEQUENCE LISTING

The Sequence Listing XML associated with this application is provided in XML file format and is hereby incorporated by reference into the specification. The name of the XML file containing the Sequence Listing XML is ROPA_028_01WO_SeqList_ST26.xml. The XML file is 156,193 bytes, and created on Apr. 19, 2023, and is being submitted electronically via USPTO Patent Center.

### BACKGROUND

Both heart failure and cardiac myopathy are serious life-threatening diseases. Loss-of-function mutations in the gene B-cell Lymphoma 2-Associated Anthanogene 3 (BAG3) have been implicated as causing cardiomyopathy e.g., dilated cardiomyopathy (DCM), including BAG3-related dilated cardiomyopathy, BAG3-related myofibrillar myopathy, familial isolated dilated cardiomyopathy, and cardiomyopathy, dilated, 1hh (CMD1HH). Downregulation of BAG3 is also associated with cardiac dysfunction and heart failure.

BAG3 mediates chaperone-assisted autophagy by serving as a cochaperone with Hsp70, blocks apoptosis by coupling with Bcl-2, preserves integrity of the sarcomere by coupling the actin filaments with the Z disc, and enhances β-agonist-stimulated excitation-contraction by linking the β-adrenergic receptor and the L-type Ca2+ channel. BAG3 downregulation disrupts the chaperone-assisted selective autophagy (CASA) complex, which mediates the turnover of cardiac proteins, such as filamin-C.

Current treatment modalities including pharmacological therapies and cardiac ablation remain ineffective for BAG3-deficient cardiomyopathy patients. There remains, therefore, an unmet need in the art for treatments for BAG3-related diseases and disorders, including cardiomyopathy and other heart conditions. The compositions and methods disclosed herein address this need.

### SUMMARY

The present invention relates generally to gene therapy vectors and gene therapy for a disease or disorder, e.g., a cardiac disease or disorder, using a vector expressing BAG3 or a functional variant thereof.

### BRIEF DESCRIPTION OF FIGURES

FIG. 1 is a diagram illustrating a non-limiting example of a vector genome. The full polynucleotide sequence of the vector genome is SEQ ID NO: 107.

FIG. 2 is a diagram illustrating a non-limiting example of a vector genome. The full polynucleotide sequence of the vector genome is SEQ ID NO: 108.

FIG. 3 is a diagram illustrating a non-limiting example of a vector genome. The full polynucleotide sequence of the vector genome is SEQ ID NO: 109.

FIG. 4 is a diagram illustrating a non-limiting example of a vector genome. The full polynucleotide sequence of the vector genome is SEQ ID NO: 110.

FIG. 5 is a diagram illustrating a non-limiting example of a vector genome. The full polynucleotide sequence of the vector genome is SEQ ID NO: 111.

FIG. 6 is a diagram illustrating a non-limiting example of a vector genome. The full polynucleotide sequence of the vector genome is SEQ ID NO: 112.

FIG. 7 is a diagram illustrating a non-limiting example of a vector genome. The full polynucleotide sequence of the vector genome is SEQ ID NO: 113.

FIG. 8 is a diagram illustrating a non-limiting example of a vector genome. The full polynucleotide sequence of the vector genome is SEQ ID NO: 114.

FIG. 9 shows expression of BAG3 in CHO-Lec2 cells following transduction by the indicated vectors. The cells were transduced with 3E6 MOI for each vector, and after 6 days, the cells lysate were collected and a Western Blot performed using an anti-BAG3 polyclonal antibody (abcam ab225561 1:2,500).

### DETAILED DESCRIPTION OF THE INVENTION

The present disclosure provides gene therapy vectors that deliver a polynucleotide encoding a BAG3 polypeptide or a functional fragment or variant thereof, along with methods of use, and other compositions and methods. In particular embodiments, the disclosure relates to a gene therapy vector comprising a promoter sequence operatively linked to a polynucleotide encoding a BAG3 polypeptide or a functional fragment or variant thereof. In certain embodiments, the promoter is a heterologous promoter, i.e., not the normal or endogenous promoter for the BAG3 gene in mammalian cells or not a normal or endogenous protein for the gene delivery vector, e.g., an AAV. In some embodiments, the BAG3 is a human BAG3.

In some embodiments, the AAV vector is an AAVrh.74 vector. In some embodiments, the promoter is an MHCK7 promoter and the AAV vector is a AAVrh.74 vector. In some embodiments, the promoter is a hTNNT2 promoter. In some embodiments, the promoter is a hTNNT2 promoter and the AAV vector is a AAVrh.74 vector. In some embodiments, the promoter is a HSP70 promoter. In some embodiments, the promoter is a HSP70 promoter and the AAV vector is a AAVrh.74 vector. In some embodiments, the promoter is a Ubiquitin C (UBC) promoter. In some embodiments, the promoter is an UBC promoter and the AAV vector is an AAVrh.74 vector. In some embodiments, the promoter is a CAG promoter or a chicken β actin promoter. In some embodiments, the promoter is a CAG promoter or a chicken β actin promoter and the AAV vector is a AAVrh.74 vector. In some embodiments, the BAG3 is human BAG3.

In some embodiments, the AAV vector is an AAV9 vector. In some embodiments, the promoter is a Myosin Heavy-chain Creatine Kinase 7 (MHCK7) promoter. In some embodiments, the promoter is an MHCK7 promoter and the AAV vector is an AAV9 vector. In some embodiments, the promoter is a hTNNT2 promoter. In some embodiments, the promoter is an hTNNT2 promoter and the AAV vector is an AAV9 vector. In some embodiments, the promoter is a heat shock protein 70 (HSP70) promoter. In some embodiments, the promoter is an HSP70 promoter and the AAV vector is an AAV9 vector. In some embodiments, the promoter is a Ubiquitin C (UBC) promoter. In some embodiments, the promoter is an UBC promoter and the AAV vector is an AAV9 vector. In some embodiments, the promoter is a CAG promoter or a chicken β actin promoter. In some embodiments, the promoter is a CAG promoter or a chicken β actin promoter and the AAV vector is an AAV9 vector. In some embodiments, the BAG3 is human BAG3.

This disclosure further provides methods of treating a disease or disorder in a subject by administering a gene therapy vector of the disclosure to the subject. In certain embodiments, the disease or disorder is heart failure. In certain embodiments, the disease or disorder is a cardiomyopathy, e.g., a dilated cardiomyopathy. In certain embodiments, the disease or disorder is BAG3-related dilated cardiomyopathy, BAG3-related myofibrillar myopathy, familial isolated dilated cardiomyopathy, and cardiomyopathy, dilated, 1hh (CMD1HH).

In certain embodiments, the subject being treated is a heart failure patient having one or more mutations or truncations in a BAG3 gene. The expression level of BAG3 is decreased in failing hearts of multiple etiologies including human heart failure. The gene BAG3 encodes the protein B-cell Lymphoma 2-Associated Anthanogene 3 (BAG3). BAG3 is involved in protein quality control (PQC). PQC comprises chaperones and protein degradation systems, including the ubiquitin-proteasome system (UPS) and autophagy-lysosome system, that help minimize proteotoxicity. Chaperones function as the first line of defense by either refolding misfolded proteins or by promoting the degradation of misfolded or damaged proteins that are not suitable for refolding via the UPS or autophagy. BAG3 regulates the ATPase activity of a class of chaperones, the HSP70 family, and is predominantly expressed in striated muscles.

BAG3 is also involved in chaperone-assisted selective autophagy (CASA). CASA is a cargo-selective form of autophagy that is mediated through the HSC70-BAG3-HSPB8 complex and allows ubiquitination of selected proteins recognized by HSC70 via the CHIP ubiquitin ligase and sequestration by autophagosomes through the LC3 adapter p62/SQSTM in the presence of synaptopodin 2 or DNAJB6. In muscles, BAG3 is particularly important for maintaining the structure and integrity of sarcomeres through its interaction with HSC70 and an actin-capping sarcomeric protein (actin-capping protein 01).

Genome wide associated studies (GWAS) have identified several mutations in BAG3, such as E455K, in patients with DCM. Additionally, mouse models (e.g., BAG3 cKO or BAG3 E455K KI) demonstrated that loss of function of BAG3 induces the development of DCM by attenuating PQC over a specific subset of proteins involved in metabolic and contractile functions in cardiomyocytes

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. In addition, the materials, methods, and examples described herein are illustrative only and are not intended to be limiting.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

As used herein, the term “in a subject over time” refers to an effect in a subject that occurs for about one day, for about one month, for about one year, for about one decade, and/or for about several decades.

As used herein, the terms “identity” and “identical” refer, with respect to a polypeptide or polynucleotide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus, a query sequence “shares at least x % identity to” a subject sequence if, when the query sequence is aligned to the subject sequence, at least x % (rounded down) of the residues in the subject sequence are aligned as an exact match to a corresponding residue in the query sequence. Where the subject sequence has variable positions (e.g., residues denoted X), an alignment to any residue in the query sequence is counted as a match. Sequence alignments may be performed using the NCBI Blast service (BLAST+ version 2.12.0).

As used herein, the term “operatively linked” refers to a functional relationship between two or more nucleic acid (e.g., DNA) segments. Typically, it refers to the functional relationship of a transcriptional regulatory sequence to a transcribed sequence. For example, a promoter sequence is operatively linked to a coding sequence if it stimulates or modulates the transcription of the coding sequence in an appropriate host cell or other expression system. Generally, promoter transcriptional regulatory sequences that are operatively linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, an “AAV vector” or “rAAV vector” refers to a recombinant vector comprising one or more polynucleotides of interest (or transgenes) that are flanked by AAV inverted terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

As used herein, “promoter” refers to a polynucleotide sequence capable of promoting initiation of RNA transcription from a polynucleotide in a eukaryotic cell.

As used herein, “vector genome” refers to the polynucleotide sequence packaged by the vector (e.g., an rAAV virion), including flanking sequences (in AAV, inverted terminal repeats). The terms “expression cassette” and “polynucleotide cassette” refer to the portion of the vector genome between the flanking ITR sequences. “Expression cassette” implies that the vector genome comprises at least one gene encoding a gene product operatively linked to an element that drives expression (e.g., a promoter).

As used herein, the term “patient in need” or “subject in need” refers to a patient or subject at risk of, or suffering from, a disease, disorder or condition that is amenable to treatment or amelioration with a recombinant gene therapy vector or gene editing system disclosed herein. A patient or subject in need may, for instance, be a patient or subject diagnosed with a disorder associated with heart. A subject may have a mutation in an BAG3 gene or deletion of all or a part of BAG3 gene, or of gene regulatory sequences, that causes aberrant interaction between BAG3 and HSP70. “Subject” and “patient” are used interchangeably herein. The subject treated by the methods described herein may be an adult or a child. Subjects may range in age.

As used herein, the term “variant” refers to a protein that has one or more amino-acid substitution, insertion, or deletion as compared to a parental protein. As used herein, the term “functional variant” refers to a protein that has one or more amino-acid substitution, insertion, or deletion as compared to a parental protein, and which retains one or more desired activities of the parental protein.

As used herein “BAG3”, “BAG3 molecules”, “BCL2-associated athanogene 3 (BAG3) genes”, “BCL2-associated athanogene 3 (BAG3) molecules” are inclusive of all family members, mutants, cDNA sequences, alleles, fragments, species, coding and noncoding sequences, sense and antisense polynucleotide strands, etc. Similarly, “BAG3”, “BAG3 molecules”, “BCL2-associated athanogene 3 (BAG3) molecules” also refer to BAG3 polypeptides or fragment thereof, proteins, variants, derivatives etc. The term “molecule” encompasses both the nucleic acid sequences and amino acid sequences of BAG3, unless specified otherwise.

As used herein, the term “fragment” refers to a fragment of a protein. As used herein, the term “functional fragment” refers to a protein that has one or more deletion as compared to a parental protein, and which retains one or more desired activities of the parental protein. In particular embodiments, a fragment may be a portion of a protein lacking an N-terminal and/or C-terminal stretch of amino acids. Fragments may comprise less than 99%, less than 95%, less than 90%, less than 80%, less than 70%, less than 60%, less than 50%, less than 40%, less than 30%, or less than 20% of the full-length protein.

As used herein, “treating” refers to ameliorating one or more symptoms of a disease or disorder. The term “preventing” refers to delaying or inhibiting or interrupting the onset of one or more symptoms of a disease or disorder or slowing the progression of BAG3-related disease or disorder, e.g., a dilated cardiomyopathy (DCM).

Adeno-associated virus (AAV) is a replication-deficient parvovirus, the single-stranded DNA genome of which is about 4.7 kb in length including two ˜145-nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAVrh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV viral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.7 kb of the genome (encoding replication and structural capsid proteins, rep-cap) may be replaced with foreign DNA. To generate AAV vectors, the rep and cap proteins may be provided in trans. Another significant feature of AAV is that it exhibits high thermal stability. It easily withstands the conditions used to inactivate adenovirus (56° to 65° C. for several hours), making cold preservation of AAV less critical. AAV may even be lyophilized. Finally, AAV-infected cells are not resistant to superinfection.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Typical examples of virus packaging cells include but are not limited to HeLa cells, SF9 cells (optionally with a baculovirus helper vector), HEK293 cells, etc. A Herpesvirus-based system can be used to produce AAV vectors, as described in US20170218395A1. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

The present disclosure contemplates compositions and methods of use related to B-cell Lymphoma 2-Associated Anthanogene 3 (BAG3) proteins or polypeptides. In certain embodiments, the subject being treated has one or more mutations in a BAG3 gene or downreguled expression or levels of BAG3 mRNA and/or protein. Mutations or downregulations in BAG3 that disrupt interaction with HSP70 is known to be associated with cardiomyopathy and heart failure, including diseases like those described in Fang et al. J Clin Invest. 127(8):3189-320 (2017) and Kieserman et al. J Am Heart Assoc. 8(10) (2019); and in other sources. Viral vector-mediated delivery of the BAG3 gene may therefore serve as a viable therapeutic for BAG3-related human diseases, such as dilated cardiomyopathy and heart failure.

Mutations in the BAG3 gene have been identified in people with BAG3-related dilated cardiomyopathy (CMD1HH). (See “CMD1HH,” NCBI MedGen). This condition is a hereditary heart disorder characterized by left ventricular or biventricular heart dilatation. The symptoms include dyspnea, syncope, collapse, fatigue, and chest pain. DCM may lead to death and can affect both children and adults. In certain embodiments, BAG3 mutations include nucleotide variant in-frame insertions as compared to wild-type BAG3 gene sequence. In certain embodiments, the in-fame insertion encodes an amino acid, e.g., a non-polar amino acid. In certain embodiments, the in-frame insertion comprises a 3-nucleotide insertion that adds an alanine at position 160.

In some embodiments, BAG3 comprises one or more amino acid substitutions. For example, BAG3 may have a C151R amino acid substitution, numbered relative to SEQ ID NO: 1 (described in Villard, E. et al. Eur Heart J. 2011 May; 32(9):1065-1076).

The BAG3 may comprise one or more amino-acid substitutions, inserts, or deletions (collectively, mutations) that alter BAG3 protein-protein binding interactions. For example, BAG3 C151R may increase in the interaction with actin-binding protein Filamin A, hippo pathway kinase STK38, and E3 ubiquitin protein ligases DDB1 and TRIM21, while interaction with small heat shock protein HSPB7 and co-chaperone DNAJB1 may decrease.

In some embodiments, the BAG3 protein comprises one or more amino acid substitutions at position C-151 to a reference BAG3 protein.

In some embodiments, BAG3 comprises one or more amino acid modifications, e.g., substitutions, including but not limited to any disclosed in PCT Patent Application Publication Nos. WO2015117010A or WO2019237002A1, U.S. Pat. No. 11,236,389, or US Patent Application Publication Nos. US20180296703 or US20210254159, which are incorporated herein by reference in their entirety.

The polypeptide and polynucleotide coding sequences of human BAG3 are shown below:

| BAG3-wild type-575 amino acids | |
|---|---|
| (SEQ ID NO: 1) | |
| MSAATHSPMMQVASGNGDRDPLPPGWEIKIDPQTGWPFFVDHNSRTTTWNDPRVPSEGPKETPSSANG | |
| PSREGSRLPPAREGHPVYPQLRPGYIPIPVLHEGAENRQVHPFHVYPQPGMQRFRTEAAAAAPQRSQS | |
| PLRGMPETTQPDKQCGQVAAAAAAQPPASHGPERSQSPAASDCSSSSSSASLPSSGRSSLGSHQLPRG | |
| YISIPVIHEQNVTRPAAQPSFHQAQKTHYPAQQGEYQTHQPVYHKIQGDDWEPRPLRAASPFRSSVQG | |
| ASSREGSPARSSTPLHSPSPIRVHTVVDRPQQPMTHRETAPVSQPENKPESKPGPVGPELPPGHIPIQ | |
| VIRKEVDSKPVSQKPPPPSEKVEVKVPPAPVPCPPPSPGPSAVPSSPKSVATEERAAPSTAPAEATPP | |
| KPGEAEAPPKHPGVLKVEAILEKVQGLEQAVDNFEGKKTDKKYLMIEEYLTKELLALDSVDPEGRADV | |
| RQARRDGVRKVQTILEKLEQKAIDVPGQVQVYELQPSNLEADQPLQAIMEMGAVAADKGKKNAGNAED | |
| PHTETQQPEATAAATSNPSSMTDTPGNPAAP | |
| BAG3-wild type-1728 nucleotide bases | |
| (SEQ ID NO: 2) | |
| atgagcgccg ccacccactc gcccatgatg caggtggcgt ccggcaacgg tgaccgcgac | 60 |
| cctttgcccc ccggatggga gatcaagatc gacccgcaga ccggctggcc cttcttcgtg | 120 |
| gaccacaaca gccgcaccac tacgtggaac gacccgcgcg tgccctctga gggccccaag | 180 |
| gagactccat cctctgccaa tggcccttcc cgggagggct ctaggctgcc gcctgctagg | 240 |
| gaaggccacc ctgtgtaccc ccagctccga ccaggctaca ttcccattcc tgtgctccat | 300 |
| gaaggcgctg agaaccggca ggtgcaccct ttccatgtct atccccagcc tgggatgcag | 360 |
| cgattccgaa ctgaggcggc agcagcggct cctcagaggt cccagtcacc tctgcggggc | 420 |
| atgccagaaa ccactcagcc agataaacag tgtggacagg tggcagcggc ggcggcagcc | 480 |
| cagcccccag cctcccacgg acctgagcgg tcccagtctc cagctgcctc tgactgctca | 540 |
| tcctcatcct cctcggccag cctgccttcc tccggcagga gcagcctggg cagtcaccag | 600 |
| ctcccgcggg ggtacatctc cattccggtg atacacgagc agaacgttac ccggccagca | 660 |
| gcccagccct ccttccacca agcccagaag acgcactacc cagcgcagca gggggagtac | 720 |
| cagacccacc agcctgtgta ccacaagatc cagggggatg actgggagcc ccggcccctg | 780 |
| cgggcggcat ccccgttcag gtcatctgtc cagggtgcat cgagccggga gggctcacca | 840 |
| gccaggagca gcacgccact ccactccccc tcgcccatcc gtgtgcacac cgtggtcgac | 900 |
| aggcctcagc agcccatgac ccatcgagaa actgcacctg tttcccagcc tgaaaacaaa | 960 |
| ccagaaagta agccaggccc agttggacca gaactccctc ctggacacat cccaattcaa | 1020 |
| gtgatccgca aagaggtgga ttctaaacct gtttcccaga agcccccacc tccctctgag | 1080 |
| aaggtagagg tgaaagttcc ccctgctcca gttccttgtc ctcctcccag ccctggccct | 1140 |
| tctgctgtcc cctcttcccc caagagtgtg gctacagaag agagggcagc ccccagcact | 1200 |
| gcccctgcag aagctacacc tccaaaacca ggagaagccg aggctccccc aaaacatcca | 1260 |
| ggagtgctga aagtggaagc catcctggag aaggtacagg ggctggagca ggctgtagac | 1320 |
| aactttgaag gcaagaagac tgacaaaaag tacctgatga tcgaagagta tttgaccaaa | 1380 |
| gagctgctgg ccctggattc agtggacccc gagggacgag ccgatgtgcg tcaggccagg | 1440 |
| agagacggtg tcaggaaggt tcagaccatc ttggaaaaac ttgaacagaa agccattgat | 1500 |
| gtcccaggtc aagtccaggt ctatgaactc cagcccagca accttgaagc agatcagcca | 1560 |
| ctgcaggcaa tcatggagat gggtgccgtg gcagcagaca agggcaagaa aaatgctgga | 1620 |
| aatgcagaag atccccacac agaaacccag cagccagaag ccacagcagc agcgacttca | 1680 |
| aaccccagca gcatgacaga cacccctggt aacccagcag caccgtag | 1728 |

In some embodiments, the BAG3 protein comprises a polypeptide sequence at least 75%, 80%, 85%, 90%, 92%, 93%, 94%, 95%, 96%, 97%, 98%, 99%, or 100% identical to SEQ ID NO: 1. In some embodiments, the BAG3 protein comprises a polynucleotide sequence at least 75%, 80%, 85%, 90%, 92%, 93%, 94%, 95%, 96%, 97%, 98%, 99%, or 100% identical to SEQ ID NO: 2. In some embodiments, the BAG3 protein is a wild-type or native BAG3 protein, e.g. human BAG3.

In some embodiments, the disclosure provides a recombinant adeno-associated virus (rAAV) virion, comprising a capsid and a vector genome, wherein the vector genome comprises a polynucleotide sequence encoding an BAG3 or a functional variant thereof, operatively linked to a promoter. In some embodiments, the disclosure provides a recombinant adeno-associated virus (rAAV) virion, comprising a capsid and a vector genome, wherein the vector genome comprises a polynucleotide sequence encoding a BAG3, operatively linked to a promoter. The polynucleotide encoding the BAG3 may comprise a polynucleotide sequence at least 75%, 80%, 85%, 90%, 92%, 93%, 94%, 95%, 96%, 97%, 98%, 99%, or 100% identical to SEQ ID NO: 2.

The AAV virions of the disclosure comprise a vector genome. The vector genome may comprise an expression cassette (or a polynucleotide cassette for gene-editing applications not requiring expression of the polynucleotide sequence).

Optionally, the polynucleotide sequence encoding the vector genome may comprise a Kozak sequence, including but not limited to GCCACCATGG (SEQ ID NO: 11). Kozak sequence may overlap the polynucleotide sequence encoding a BAG3 protein or a functional variant thereof.

In some embodiments, the Kozak sequence is an alternative Kozak sequence comprising or consisting of any one of:

(SEQ ID NO: 13)
(gcc)gccRccAUGG;
(SEQ ID NO: 45)
AGNNAUGN;
(SEQ ID NO: 46)
ANNAUGG;
(SEQ ID NO: 47)
ANNAUGC;
(SEQ ID NO: 49)
ACCAUGG;,
and
(SEQ ID NO: 14)
GACACCAUGG,

or having at least 80%, at least 85%, at least 90%, at least 95%, or at least 98% to any of SEQ ID NOs: 13, 45, 46, 47, 49 or 14.

In some embodiments, the vector genome comprises no Kozak sequence.

The vector genome may comprise 5′ and/or 3′ inverted terminal repeats (ITRs). Any suitable ITRs may be used. The ITRs may be AAV ITRs from the same serotype as the capsid present in the AAV virion, or a different serotype from the capsid (e.g., AAV2 ITRs may be used with an AAV virion having an AAV9 capsid or an AAVrh74 capsid). In each case, the serotype of the capsid determines the name applied to the virion. The ITR are generally the most 5′ and most 3′ elements of the vector genome. The vector genome will also generally contain, in 5′ to 3′ order, a promoter, a transgene, 3′ untranslated region (UTR) sequences (e.g., a WPRE element), and a polyadenylation sequence. In variations, the vector genome includes an enhancer element (generally 5′ to the promoter) and/or an exon (generally 3′ to the promoter). In variations, the vector genome includes a Green Fluorescence Protein (GFP) protein, generally 3′ to the transgene. In variations, the vector genomes of the disclosure encode a partial or complete transgene sequence used as a repair template in a gene editing system. In such variations, the vector genome may comprise an exogenous promoter, or the gene editing system may insert the transgene into a locus in the genome having an endogenous promoter, such as a cardiac- or myocyte-specific promoter.

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 15.

| SEQ ID NO: 15 | |
|---|---|
| cctgcaggca gctgcgcgct cgctcgctca ctgaggccgc ccgggcaaag cccgggcgtc | 60 |
| gggcgacctt tggtcgcccg gcctcagtga gcgagcgagc gcgcagagag ggagtggcca | 120 |
| actccatcac taggggttcc t | 141 |

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 16.

| SEQ ID NO: 16 | |
|---|---|
| gcgcgctcgc tcgctcactg aggccgcccg ggcaaagccc gggcgtcggg cgacctttgg | 60 |
| tcgcccggcc tcagtgagcg agcgagcgcg cagagaggga gtggccaact ccatcactag | 120 |
| gggttccttg tagttaatga ttaacccgcc atgctactta tctacgta | 168 |

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 17)

| SEQ ID NO: 17 | |
|---|---|
| ctgcgcgctc gctcgctcac tgaggccgcc cgggcaaagc ccgggcgtcg ggcgaccttt | 60 |
| ggtcgcccgg cctcagtgag cgagcgagcg cgcagagagg gagtggccaa ctccatcact | 120 |
| aggggttcct tgtagttaat gattaacccg ccatgctact tatctacgta | 170 |

In some embodiments, the 5′ ITR comprises a polynucleotide sequence at least 75%, 80%, 85%, 90%, 92%, 93%, 94%, 95%, 96%, 97%, 98%, 99%, or 100% identical to SEQ ID NO: 18.

| SEQ ID NO: 18 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcct | 145 |

In some embodiments, the 3′ ITR comprises a polynucleotide sequence at least 75%, 80%, 85%, 90%, 92%, 93%, 94%, 95%, 96%, 97%, 98%, 99%, or 100% identical to SEQ ID NO: 19.

| SEQ ID NO: 19 | |
|---|---|
| aggaacccct agtgatggag ttggccactc cctctctgcg cgctcgctcg ctcactgagg | 60 |
| ccgggcgacc aaaggtcgcc cgacgcccgg gctttgcccg ggcggcctca gtgagcgagc | 120 |
| gagcgcgcag ctgcctgcag g | 141 |

In some embodiments, the 3′ ITR comprises a polynucleotide sequence at least 75%, 80%, 85%, 90%, 92%, 93%, 94%, 95%, 96%, 97%, 98%, 99%, or 100% identical to SEQ ID NO: 20.

| SEQ ID NO: 20 | |
|---|---|
| tacgtagata agtagcatgg cgggttaatc attaactaca aggaacccct agtgatggag | 60 |
| ttggccactc cctctctgcg cgctcgctcg ctcactgagg ccgggcgacc aaaggtcgcc | 120 |
| cgacgcccgg gctttgcccg ggcggcctca gtgagcgagc gagcgcgc | 168 |

In some embodiments, the 3′ ITR comprises a polynucleotide sequence at least 75%, 80%, 85%, 90%, 92%, 93%, 94%, 95%, 96%, 97%, 98%, 99%, or 100% identical to SEQ ID NO: 21.

| SEQ ID NO: 21 | |
|---|---|
| aggaacccct agtgatggag ttggccactc cctctctgcg cgctcgctcg ctcactgagg | 60 |
| ccgggcgacc aaaggtcgcc cgacgcccgg gctttgcccg ggcggcctca gtgagcgagc | 120 |
| gagcgcgcag agagggagtg gccaa | 145 |

In some embodiments the vector genome comprises one or more filler sequences, e.g., at least 75%, 80%, 85%, 90%, 92%, 93%, 94%, 95%, 96%, 97%, 98%, 99%, or 100% identical to SEQ ID NO: 22; SEQ ID NO: 23; or SEQ ID NO: 24.

| SEQ ID NO: 22 | |
|---|---|
| gcggcaattc agtcgataac tataacggtc ctaaggtagc gatttaaata cgcgctctct | 60 |
| taaggtagcc ccgggacgcg tcaattgact acaaaccgag tatctgcaga gggccctgcg | 120 |
| tatg | 124 |
| SEQ ID NO: 23 | |
| cttctgaggc ggaaagaacc agatcctctc ttaaggtagc atcgagattt aaattaggga | 60 |
| taacagggta atggcgcggg ccgc | 84 |
| SEQ ID NO: 24 | |
| gttacccagg ctggagtgca gtggcacatt tctgctcact gcaacctcct cctccctggg | 60 |
| ttc | 63 |

In some embodiments, the polynucleotide sequence encoding an BAG3 protein or functional variant thereof is operatively linked to a promoter. In some embodiments, the promoter is an MHCK7 promoter. In some embodiments, the promoter is an TNNT2 promoter. In some embodiments, the promoter is a HSP70 promoter. In some embodiments, the promoter is a UBC promoter. In some embodiments, the promoter is a CAG promoter or a chicken β-actin promoter.

The present disclosure contemplates use of various promoters. Promoters useful in embodiments of the present disclosure include, without limitation, a cytomegalovirus (CMV) promoter, phosphoglycerate kinase (PGK) promoter, or a promoter sequence comprised of the CMV enhancer and portions of the chicken beta-actin promoter and the rabbit beta-globin gene (CAG). In some cases, the promoter may be a synthetic promoter. Exemplary synthetic promoters are provided by Schlabach et al. PNAS USA. 107(6):2538-43 (2010). In some embodiments, the promoter comprises a polynucleotide sequence at least 75%, 80%, 85%, 90%, 92%, 93%, 94%, 95%, 96%, 97%, 98%, 99%, or 100% identical to SEQ ID NO: 25.

| SEQ ID NO: 25. | |
|---|---|
| 60 | acttacggta aatggcccgc ctggctgacc gcccaacgac ccccgcccat tgacgtcaat |
| 120 | aatgacgtat gttcccatag taacgccaat agggactttc cattgacgtc aatgggtgga |
| 180 | gtatttacgg taaactgccc acttggcagt acatcaagtg tatcatatgc caagtacgcc |
| 240 | ccctattgac gtcaatgacg gtaaatggcc cgcctggcat tatgcccagt acatgacctt |
| 300 | atgggacttt cctacttggc agtacatcta cgtattagtc atcgctatta ccatggtcga |
| 360 | ggtgagcccc acgttctgct tcactctccc catctccccc ccctccccac ccccaatttt |
| 420 | gtatttattt attttttaat tattttgtgc agcgatgggg gcgggggggg ggggggcgcg |
| 480 | cgccaggcgg ggcggggcgg ggcgaggggc ggggcggggc gaggcggaga ggtgcggcgg |
| 540 | cagccaatca gagcggcgcg ctccgaaagt ttccttttat ggcgaggcgg cggcggcggc |
| 573 | ggccctataa aaagcgaagc gcgcggcggg cgg |

In some embodiments, a polynucleotide sequence encoding an BAG3 protein or functional variant thereof is operatively linked to an inducible promoter. An inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). Inducible promoters include heavy metal ion inducible promoters (such as the mouse mammary tumor virus (mMTV) promoter or various growth hormone promoters), and the promoters from T7 phage which are active in the presence of T7 RNA polymerase. This list of inducible promoters is non-limiting.

In some cases, the promoter is a tissue-specific promoter, such as a promoter capable of driving expression in a cardiac cell to a greater extent than in a non-cardiac cell. In some embodiments, tissue-specific promoter is a selected from any various cardiac tissue- or cell-specific promoters including but not limited to, desmin (Des), alpha-myosin heavy chain (α-MHC), myosin light chain 2 (MLC-2), cardiac troponin C (cTnC), cardiac troponin T (hTNNT2), muscle creatine kinase (CK) and combinations of promoter/enhancer regions thereof, such as MHCK7. In some embodiments, the promoter is inducible to environmental stimuli. In some embodiments, the promoter is a heat shock protein 70 (HSP70) promoter. In some cases, the promoter is a ubiquitous promoter. A “ubiquitous promoter” refers to a promoter that is not tissue-specific under experimental or clinical conditions. In some cases, the ubiquitous promoter is any one of Cytomegalovirus (CMV), Cytomegalovirus early enhancer element chicken beta-Actin gene intron with the splice acceptor of the rabbit beta-Globin gene (CAG), ubiquitin C (UBC), Phosphoglycerate Kinase (PGK), Eukaryotic translation elongation factor 1 alpha 1 (EF1-alpha), Glyceraldehyde 3-phosphate dehydrogenase (GAPDH), simian virus 40 (SV40), Hepatitis B virus (HBV), chicken beta-actin, and human beta-actin promoters.

In some embodiments, the promoter sequence is selected from Table 1. In some embodiments, the promoter comprises a polynucleotide sequence at least 75%, 80%, 85%, 90%, 92%, 93%, 94%, 95%, 96%, 97%, 98%, 99%, or 100% identical to any one of SEQ ID NOS: 31-48, 115 or 116. In some embodiments, the promoter comprises a fragment of a polynucleotide sequence of any one of SEQ ID NOs: 31-48, e.g., a fragment comprising at least 25%, at least 50%, at least 75%, 80%, 85%, 90%, 92%, 93%, 94%, 95%, 96%, 97%, 98%, 99%, or 100% of any one of SEQ ID NOs: 31-51, 115 or 116. For example, in some embodiments, the promoter is a fragment of the UBC promoter consisting of about the first 400 nucleotides of the UBC promoter sequence of SEQ TD NO: 116.

TABLE 1

| | | SEQ ID |
|---|---|---|
| PROMOTER | SEQUENCE | NO: |
| MHCK7 | ACCCTTCAGATTAAAAATAACTGAGGTAAGGGCCTGGGTAG | 31 |
| | GGGAGGTGGTGTGAGACGCTCCTGTCTCTCCTCTATCTGCCC | |
| | ATCGGCCCTTTGGGGAGGAGGAATGTGCCCAAGGACTAAAA | |
| | AAAGGCCATGGAGCCAGAGGGGCGAGGGCAACAGACCTTTC | |
| | ATGGGCAAACCTTGGGGCCCTGCTGTCTAGCATGCCCCACTA | |
| | CGGGTCTAGGCTGCCCATGTAAGGAGGCAAGGCCTGGGGAC | |
| | ACCCGAGATGCCTGGTTATAATTAACCCAGACATGTGGCTGC | |
| | CCCCCCCCCCCCAACACCTGCTGCCTCTAAAAATAACCCTGT | |
| | CCCTGGTGGATCCCCTGCATGCGAAGATCTTCGAACAAGGCT | |
| | GTGGGGGACTGAGGGCAGGCTGTAACAGGCTTGGGGGCCAG | |
| | GGCTTATACGTGCCTGGGACTCCCAAAGTATTACTGTTCCAT | |
| | GTTCCCGGCGAAGGGCCAGCTGTCCCCCGCCAGCTAGACTCA | |
| | GCACTTAGTTTAGGAACCAGTGAGCAAGTCAGCCCTTGGGGC | |
| | AGCCCATACAAGGCCATGGGGCTGGGCAAGCTGCACGCCTG | |
| | GGTCCGGGGTGGGCACGGTGCCCGGGCAACGAGCTGAAAGC | |
| | TCATCTGCTCTCAGGGGCCCCTCCCTGGGGACAGCCCCTCCT | |
| | GGCTAGTCACACCCTGTAGGCTCCTCTATATAACCCAGGGGC | |
| | ACAGGGGCTGCCCTCATTCTACCACCACCTCCACAGCACAGA | |
| | CAGACACTCAGGAGCCAGCCAG | |
| Human cardiac | CTCAGTCCATTAGGAGCCAGTAGCCTGGAAGATGTCTTTACC | 33 |
| troponin T | CCCAGCATCAGTTCAAGTGGAGCAGCACATAACTCTTGCCCT | |
| promoter | CTGCCTTCCAAGATTCTGGTGCTGAGACTTATGGAGTGTCTT | |
| (without exon 1) | GGAGGTTGCCTTCTGCCCCCCAACCCTGCTCCCAGCTGGCCC | |
| hTnnT2/ | TCCCAGGCCTGGGTTGCTGGCCTCTGCTTTATCAGGATTCTCA | |
| HTNNT2 | AGAGGGACAGCTGGTTTATGTTGCATGACTGTTCCCTGCATA | |
| | TCTGCTCTGGTTTTAAATAGCTTATCTGAGCAGCTGGAGGAC | |
| | CACATGGGCTTATATGGCGTGGGGTACATGTTCCTGTAGCCT | |
| | TGTCCCTGGCACCTGCCAAAATAGCAGCCAACACCCCCCACC | |
| | CCCACCGCCATCCCCCTGCCCCACCCGTCCCCTGTCGCACAT | |
| | TCCTCCCTCCGCAGGGCTGGCTCACCAGGCCCCAGCCCACAT | |
| | GCCTGCTTAAAGCCCTCTCCATCCTCTGCCTCACCCAGT | |
| Human cardiac | CTCAGTCCATTAGGAGCCAGTAGCCTGGAAGATGTCTTTACC | 32 |
| troponin T | CCCAGCATCAGTTCAAGTGGAGCAGCACATAACTCTTGCCCT | |
| promoter (with | CTGCCTTCCAAGATTCTGGTGCTGAGACTTATGGAGTGTCTT | |
| exon 1, | GGAGGTTGCCTTCTGCCCCCCAACCCTGCTCCCAGCTGGCCC | |
| underlined) | TCCCAGGCCTGGGTTGCTGGCCTCTGCTTTATCAGGATTCTCA | |
| hTnnT2/ | AGAGGGACAGCTGGTTTATGTTGCATGACTGTTCCCTGCATA | |
| HTNNT2 | TCTGCTCTGGTTTTAAATAGCTTATCTGAGCAGCTGGAGGAC | |
| | CACATGGGCTTATATGGCGTGGGGTACATGTTCCTGTAGCCT | |
| | TGTCCCTGGCACCTGCCAAAATAGCAGCCAACACCCCCCACC | |
| | CCCACCGCCATCCCCCTGCCCCACCCGTCCCCTGTCGCACAT | |
| | TCCTCCCTCCGCAGGGCTGGCTCACCAGGCCCCAGCCCACAT | |
| | GCCTGCTTAAAGCCCTCTCCATCCTCTGCCTCACCCAGTCCCC | |
| | GCTGAGACTGAGCAGACGCCTCCAGGATCTGTCGGCAG | |
| Mouse α- | GGTACCGGATCCTGCAAGGTCACACAAGGGTCTCCACCCACC | 34 |
| cardiac myosin | AGGTGCCCTAGTCTCAATTTCAGTTTCCATGCCTTGTTCTCAC | |
| heavy chain | AATGCTGGCCTCCCCAGAGCTAATTTGGACTTTGTTTTTATTT | |
| promoter | CAAAAGGGCCTGAATGAGGAGTAGATCTTGTGCTACCCAGC | |
| (αMHC) | TCTAAGGGTGCCCGTGAAGCCCTCAGACCTGGAGCCTTTGCA | |
| | ACAGCCCTTTAGGTGGAAGCAGAATAAAGCAATTTTCCTTAA | |
| | AGCCAAAATCCTGCCTCTAGACTCTTCTTCTCTGACCTCGGTC | |
| | CCTGGGCTCTAGGGTGGGGAGGTGGGGCTTGGAAGAAGAAG | |
| | GTGGGGAAGTGGCAAAAGCCGATCCCTAGGGCCCTGTGAAG | |
| | TTCGGAGCCTTCCCTGTACAGCACTGGCTCATAGATCCTCCT | |
| | CCAGCCAAACATAGCAAGAAGTGATACCTCCTTTGTGACTTC | |
| | CCCAGGCCCAGTACCTGTCAGGTTGAAACAGGATTTAGAGA | |
| | AGCCTCTGAACTCACCTGAACTCTGAAGCTCATCCACCAAGC | |
| | AAGCACCTAGGTGCCACTGCTAGTTAGTATCCTACGCTGATA | |
| | ATATGCAGAGCTGGGCCACAGAAGTCCTGGGGTGTAGGAAC | |
| | TGACCAGTGACTTTTCAGTCGGCAAAGGTATGACCCCCTCAG | |
| | CAGATGTAGTAATGTCCCCTTAGATCCCATCCCAGGCAGGTC | |
| | TCTAAGAGGACATGGGATGAGAGATGTAGTCATGTGGCATT | |
| | CCAAACACAGCTATCCACAGTGTCCCTTGCCCCTTCCACTTA | |
| | GCCAGGAGGACAGTAACCTTAGCCTATCTTTCTTCCTCCCCA | |
| | TCCTCCCAGGACACACCCCCTGGTCTGCAGTATTCATTTCTTC | |
| | CTTCACGTCCCCTCTGTGACTTCCATTTGCAAGGCTTTTGACC | |
| | TCTGCAGCTGCTGGAAGATAGAGTTTGGCCCTAGGTGTGGCA | |
| | AGCCATCTCAAGAGAAAGCAGACAACAGGGGGACCAGATTT | |
| | TGGAAGGATCAGGAACTAAATCACTGGCGGGCCTGGGGGTA | |
| | GAAAAAAGAGTGAGTGAGTCCGCTCCAGCTAAGCCAAGCTA | |
| | GTCCCCGAGATACTCTGCCACAGCTGGGCTGCTCGGGGTAGC | |
| | TTTAGGAATGTGGGTCTGAAAGACAATGGGATTGGAAGACA | |
| | TCTCTTTGAGTCTCCCCTCAACCCCACCTACAGACACACTCGT | |
| | GTGTGGCCAGACTCCTGTTCAACAGCCCTCTGTGTTCTGACC | |
| | ACTGAGCTAGGCAACCAGAGCATGGGCCCTGTGCTGAGGAT | |
| | GAAGAGTTGGTTACCAATAGCAAAAACAGCAGGGGAGGGAG | |
| | AACAGAGAACGAAATAAGGAAGGAAGAAGGAAAGGCCAGT | |
| | CAATCAGATGCAGTCAGAAGAGATGGGAAGCCAACACACAG | |
| | CTTGAGCAGAGGAAACAGAAAAGGGAGAGATTCTGGGCATA | |
| | AGGAGGCCACAGAAAGAAGAGCCCAGGCCCCCCAAGTCTCC | |
| | TCTTTATACCCTCATCCCGTCTCCCAATTAAGCCCACTCTTCT | |
| | TCCTAGATCAGACCTGAGCTGCAGCGAAGAGACCCGTAGGG | |
| | AGGATCACACTGGATGAAGGAGATGTGTGGAGAAGTCCAGG | |
| | GAACCTAAGAGCCAGAGCCTAAAAGAGCAAGAGATAAAGGT | |
| | GCTTCAAAGGTGGCCAGGCTGTGCACACAGAGGGTCGAGGA | |
| | CTGGTGGTAGAGCCTCAAGATAAGGATGATGCTCAGAATGG | |
| | GCGGGGGGGGGGATTCTGGGGGGGGGAGAGAGAAGGTGAG | |
| | AAGGAGCCTGGAACAGAGAATCTGGAAGCGCTGGAAACGAT | |
| | ACCATAAAGGGAAGAACCCAGGCTACCTTTAGATGTAAATC | |
| | ATGAAAGACAGGGAGAAGGGAAGCTGGAGAGAGTAGAAGG | |
| | ACCCCGGGGCAAGACATTGAAGCAAGGACAAGCCAGGTTGA | |
| | GCGCTCCGTGAAATCAGCCTGCTGAAGGCAGAGCCCTGGTAT | |
| | GAGCACCAGAACAGCAGAGGCTAGGGTTAATGTCGAGACAG | |
| | GGAACAGAAGGTAGACACAGGAACAGACAGAGACGGGGGA | |
| | GCCAGGTAACAAAGGAATGGTCCTTCTCACCTGTGGCCAGA | |
| | GCGTCCATCTGTGTCCACATACTCTAGAATGTTCATCAGACT | |
| | GCAGGGCTGGCTTGGGAGGCAGCTGGAAAGAGTATGTGAGA | |
| | GCCAGGGGAGACAAGGGGGCCTAGGAAAGGAAGAAGAGGG | |
| | CAAACCAGGCCACACAAGAGGGCAGAGCCCAGAACTGAGTT | |
| | AACTCCTTCCTTGTTGCATCTTCCATAGGAGGCAGTGGGAAC | |
| | TCTGTGACCACCATCCCCCATGAGCCCCCACTACCCATACCA | |
| | AGTTTGGCCTGAGTGGCATTCTAGGTTCCCTGAGGACAGAGC | |
| | CTGGCCTTTGTCTCTTGGACCTGACCCAAGCTGACCCAATGT | |
| | TCTCAGTACCTTATCATGCCCTCAAGAGCTTGAGAACCAGGC | |
| | AGTGACATATTAGGCCATGGGCTAACCCTGGAGCTTGCACAC | |
| | AGGAGCCTCAAGTGACCTCCAGGGACACAGCTGCAGACAGG | |
| | TGGCCTTTATCCCCAAAGAGCAACCATTTGGCATAGGTGGCT | |
| | GCAAATGGGAATGCAAGGTTGAATCAGGTCCCTTCAAGAAT | |
| | ACTGCATGCAAGACCTAAGACCCCTGGAGAGAGGGGTATGC | |
| | TCCTGCCCCCACCCACCATAAGGGGAGTGAACTATCCTAGGG | |
| | GGCTGGCGACCTTGGGGAGACACCACATTACTGAGAGTGCT | |
| | GAGCCCAGAAAAACTGACCGCCCTGTGTCCTGCCCACCTCCA | |
| | CACTCTAGAGCTATATTGAGAGGTGACAGTAGATAGGGTGG | |
| | GAGCTGGTAGCAGGGAGAGTGTTCCTGGGTGTGAGGGTGTA | |
| | GGGGAAAGCCAGAGCAGGGGAGTCTGGCTTTGTCTCCTGAA | |
| | CACAATGTCTACTTAGTTATAACAGGCATGACCTGCTAAAGA | |
| | CCCAACATCTACGACCTCTGAAAAGACAGCAGCCCTGGAGG | |
| | ACAGGGGTTGTCTCTGAGCCTTGGGTGCTTGATGGTGCCACA | |
| | AAGGAGGGCATGAGTGTGAGTATAAGGCCCCAGGAGCGTTA | |
| | GAGAAGGGCACTTGGGAAGGGGTCAGTCTGCAGAGCCCCTA | |
| | TCCATGGAATCTGGAGCCTGGGGCCAACTGGTGTAAATCTCT | |
| | GGGCCTGCCAGGCATTCAAAGCAGCACCTGCATCCTCTGGCA | |
| | GCCTGGGGAGGCGGAAGGGAGCAACCCCCCACTTATACCCT | |
| | TTCTCCCTCAGCCCCAGGATTAACACCTCTGGCCTTCCCCCTT | |
| | CCCACCTCCCATCAGGAGTGGAGGGTTGCAGAGGGAGGGTA | |
| | AAAACCTACATGTCCAAACATCATGGTGCACGATATATGGAT | |
| | CAGTATGTGTAGAGGCAAGAAAGGAAATCTGCAGGCTTAAC | |
| | TGGGTTAATGTGTAAAGTCTGTGTGCATGTGTGTGTGTCTGA | |
| | CTGAAAACGGGCATGGCTGTGCAGCTGTTCAGTTCTGTGCGT | |
| | GAGGTTACCAGACTGCAGGTTTGTGTGTAAATTGCCCAAGGC | |
| | AAAGTGGGTGAATCCCTTCCATGGTTTAAAGAGATTGGATGA | |
| | TGGCCTGCATCTCAAGGACCATGGAAAATAGAATGGACACT | |
| | CTATATGTGTCTCTAAGCTAAGGTAGCAAGGTCTTTGGAGGA | |
| | CACCTGTCTAGAGATGTGGGCAACAGAGACTACAGACAGTA | |
| | TCTGTACAGAGTAAGGAGAGAGAGGAGGGGGTGTAGAATTC | |
| | TCTTACTATCAAAGGGAAACTGAGTCGTGCACCTGCAAAGTG | |
| | GATGCTCTCCCTAGACATCATGACTTTGTCTCTGGGGAGCCA | |
| | GCACTGTGGAACTTCAGGTCTGAGAGAGTAGGAGGCTCCCCT | |
| | CAGCCTGAAGCTATGCAGATAGCCAGGGTTGAAAGGGGGAA | |
| | GGGAGAGCCTGGGATGGGAGCTTGTGTGTTGGAGGCAGGGG | |
| | ACAGATATTAAGCCTGGAAGAGAAGGTGACCCTTACCCAGT | |
| | TGTTCAACTCACCCTTCAGATTAAAAATAACTGAGGTAAGGG | |
| | CCTGGGTAGGGGAGGTGGTGTGAGACGCTCCTGTCTCTCCTC | |
| | TATCTGCCCATCGGCCCTTTGGGGAGGAGGAATGTGCCCAAG | |
| | GACTAAAAAAAGGCCATGGAGCCAGAGGGGCGAGGGCAAC | |
| | AGACCTTTCATGGGCAAACCTTGGGGCCCTGCTGTCCTCCTG | |
| | TCACCTCCAGAGCCAAGGGATCAAAGGAGGAGGAGCCAGGA | |
| | CAGGAGGGAAGTGGGAGGGAGGGTCCCAGCAGAGGACTCC | |
| | AAATTTAGGCAGCAGGCATATGGGATGGGATATAAAGGGGC | |
| | TGGAGCACTGAGAGCTGTCAGAGATTTCTCCAACCCAGGTAA | |
| | GAGGGAGTTTCGGGTGGGGGCTCTTCACCCACACCAGACCTC | |
| | TCCCCACCTAGAAGGAAACTGCCTTTCCTGGAAGTGGGGTTC | |
| | AGGCCGGTCAGAGATCTGACAGGGTGGCCTTCCACCAGCCT | |
| | GGGAAGTTCTCAGTGGCAGGAGGTTTCCACAAGAAACACTG | |
| | GATGCCCCTTCCCTTACGCTGTCTTCTCCATCTTCCTCCTGGG | |
| | GATGCTCCTCCCCGTCTTGGTTTATCTTGGCTCTTCGTCTTCA | |
| | GCAAGATTTGCCCTGTGCTGTCCACTCCATCTTTCTCTACTGT | |
| | CTCCGTGCCTTGCCTTGCCTTCTTGCGTGTCCTTCCTTTCCAC | |
| | CCATTTCTCACTTCACCTTTTCTCCCCTTCTCATTTGTATTCAT | |
| | CCTTCCTTCCTTCCTTCCTTCCTTCCTTCCTTCCTTCCTTCCTTC | |
| | CTTTCTCCCTTCCTTCCTTCCTTCCTTCCTTCCTTCCTTCCTTCC | |
| | TTCCTGTGTCAGAGTGCTGAGAATCACACCTGGGGTTCCCAC | |
| | CCTTATGTAAACAATCTTCCAGTGAGCCACAGCTTCAGTGCT | |
| | GCTGGGTGCTCTCTTACCTTCCTCACCCCCTGGCTTGTCCTGT | |
| | TCCATCCTGGTCAGGATCTCTAGATTGGTCTCCCAGCCTCTGC | |
| | TACTCCTCTTCCTGCCTGTTCCTCTCTCTGTCCAGCTGCGCCA | |
| | CTGTGGTGCCTCGTTCCAGCTGTGGTCCACATTCTTCAGGATT | |
| | CTCTGAAAAGTTAACCAGGTGAGAATGTTTCCCCTGTAGACA | |
| | GCAGATCACGATTCTCCCGGAAGTCAGGCTTCCAGCCCTCTC | |
| | TTTCTCTGCCCAGCTGCCCGGCACTCTTAGCAAACCTCAGGC | |
| | ACCCTTACCCCACATAGACCTCTGACAGAGAAGCAGGCACTT | |
| | TACATGGAGTCCTGGTGGGAGAGCCATAGGCTACGGTGTAA | |
| | AAGAGGCAGGGAAGTGGTGGTGTAGGAAAGTCAGGACTTCA | |
| | CATAGAAGCCTAGCCCACACCAGAAATGACAGACAGATCCC | |
| | TCCTATCTCCCCCATAAGAGTTTGAGTCGACCCGCGGCCCCG | |
| | AATTG | |
| Chicken cardiac | GGGATAAAAGCAGTCTGGGCTTTCACATGACAGCATCTGGG | 35 |
| troponin T | GCTGCGGCAGAGGGTCGGGTCCGAAGCGCTGCCTTATCAGC | |
| promoter | GTCCCCAGCCCTGGGAGGTGACAGCTGGCTGGCTTGTGTCAG | |
| (cTnT) | CCCCTCGGGCACTCACGTATCTCCGTCCGACGGGTTTAAAAT | |
| | AGCAAAACTCTGAGGCCACACAATAGCTTGGGCTTATATGG | |
| | GCTCCTGTGGGGGAAGGGGGAGCACGGAGGGGGCCGGGGCC | |
| | GCTGCTGCCAAAATAGCAGCTCACAAGTGTTGCATTCCTCTC | |
| | TGGGCGCCGGGCACATTCCTGCTGGCTCTGCCCGCCCCGGGG | |
| | TGGGCGCCGGGGGGACCTTAAAGCCTCTGCCCCCCAAGGAG | |
| | CCCTTCCCAGACAGCCGCCGGCACCCACCGCTCCGTGGGA | |
| Human Creatine | CTCTCAGCCCTGGAAGTCCTTGCTCACAGCCGAGGCGCCGAG | 36 |
| Kinase M | AGCGCTTGCTCTGCCCAGATCTGCGCGAGTCTGGCGCCCGCG | |
| (hCKM) | CTCTGAACGGCGTCGCTGCCCAGCCCCCTTCCCCGGGAGGTG | |
| | GGAGCGGCCACCCAGGGCCCCGTGGCTGCCCTTGTAAGGAG | |
| | GCGAGGCCCGAGGACACCCGAGACGCCCGGTTATAATTAAC | |
| | CAGGACACGTGGCGAACCCCCCTCCAACACCTGCCCCCGAA | |
| | CCCCCCCATACCCAGCGCCTCGGGTCTCGGCCTTTGCGGCAG | |
| | AGGAGACAGCAAAGCGCCCTCTAAAAATAACTCCTTTCCCG | |
| | GCGACCGAGACCCTCCCTGTCCCCCGCACAGCGGAAATCTCC | |
| | CAGTGGCACCGAGGGGGCGAGGGTTAAGTGGGGGGGAGGGT | |
| | GACCACCGCCTCCCACCCTTGCCCTGAGTTTGAATCTCTCCA | |
| | ACTCAGCCAGCCTCAGTTTCCCCTCCACTCAGTCCCTAGGAG | |
| | GAAGGGGCGCCCAAGCGCGGGTTTCTGGGGTTAGACTGCCC | |
| | TCCATTGCAATTGGTCCTTCTCCCGGCCTCTGCTTCCTCCAGC | |
| | TCACAGGGTATCTGCTCCTCCTGGAGCCACACCTTGGTTCCC | |
| | CGAGGTGCCGCTGGGACTCGGGTAGGGGTGAGGGCCCAGGG | |
| | GGCACAGGGGGAGCCGAGGGCCACAGGAAGGGCTGGTGGCT | |
| | GAAGGAGACTCAGGGGCCAGGGGACGGTGGCTTCTACGTGC | |
| | TTGGGACGTTCCCAGCCACCGTCCCATGTTCCCGGCGGGGGG | |
| | CCAGCTGTCCCCACCGCCAGCCCAACTCAGCACTTGGTCAGG | |
| | GTATCAGCTTGGTGGGGGGGCGTGAGCCCAGCCCCTGGGGC | |
| | GGCTCAGCCCATACAAGGCCATGGGGCTGGGCGCAAAGCAT | |
| | GCCTGGGTTCAGGGTGGGTATGGTGCGGGAGCAGGGAGGTG | |
| | AGAGGCTCAGCTGCCCTCCAGAACTCCTCCCTGGGGACAACC | |
| | CCTCCCAGCCAATAGCACAGCCTAGGTCCCCCTATATAAGGC | |
| | CACGGCTGCTGGCCCTTCCTTTGGGTCAGTGTCACCTCCAGG | |
| | ATACAGACA | |
| Human beta- | GCCCAGCACCCCAAGGCGGCCAACGCCAAAACTCTCCCTCCT | 37 |
| actin (HuBa) | CCTCTTCCTCAATCTCGCTCTCGCTCTTTTTTTTTTTCGCAAAA | |
| | GGAGGGGAGAGGGGGTAAAAAAATGCTGCACTGTGCGGCGA | |
| | AGCCGGTGAGTGAGCGGCGCGGGGCCAATCAGCGTGCGCCG | |
| | TTCCGAAAGTTGCCTTTTATGGCTCGAGCGGCCGCGGCGGCG | |
| | CCCTATAAAACCCAGCGGCGCGACGCGCCACCACCGCCGAG | |
| | TC | |
| Chicken beta- | GGTCGAGGTGAGCCCCACGTTCTGCTTCACTCTCCCCATCTC | 38 |
| actin (CBA) | CCCCCCCTCCCCACCCCCAATTTTGTATTTATTTATTTTTTAAT | |
| | TATTTTGTGCAGCGATGGGGGCGGGGGGGGGGGGGGCGCGC | |
| | GCCAGGCGGGGCGGGGCGGGGCGAGGGGCGGGGCGGGGCG | |
| | AGGCGGAGAGGTGCGGCGGCAGCCAATCAGAGCGGCGCGCT | |
| | CCGAAAGTTTCCTTTTATGGCGAGGCGGCGGCGGCGGCGGCC | |
| | CTATAAAAAGCGAAGCGCGCGGCGGGCGGGA | |
| Cytomegalovirus | TGGTGATGCGGTTTTGGCAGTACACCAATGGGCGTGGATAGC | 39 |
| (CMV) | GGTTTGACTCACGGGGATTTCCAAGTCTCCACCCCATTGACG | |
| | TCAATGGGAGTTTGTTTTGGCACCAAAATCAACGGGACTTTC | |
| | CAAAATGTCGTAATAACCCCGCCCCGTTGACGCAAATGGGC | |
| | GGTAGGCGTGTACGGTGGGAGGTCTATATAAGCAGAGCTCG | |
| | TTTAGTGAACCG | |
| Cytomegalovirus | TAGTTATTAATAGTAATCAATTACGGGGTCATTAGTTCATAG | 40 |
| (CMV) | CCCATATATGGAGTTCCGCGTTACATAACTTACGGTAAATGG | |
| (second version) | CCCGCCTGGCTGACCGCCCAACGACCCCCGCCCATTGACGTC | |
| | AATAATGACGTATGTTCCCATAGTAACGCCAATAGGGACTTT | |
| | CCATTGACGTCAATGGGTGGAGTATTTACGGTAAACTGCCCA | |
| | CTTGGCAGTACATCAAGTGTATCATATGCCAAGTACGCCCCC | |
| | TATTGACGTCAATGACGGTAAATGGCCCGCCTGGCATTATGC | |
| | CCAGTACATGACCTTATGGGACTTTCCTACTTGGCAGTACAT | |
| | CTACGTATTAGTCATCGCTATTACCATGGTGATGCGGTTTTG | |
| | GCAGTACATCAATGGGCGTGGATAGCGGTTTGACTCACGGG | |
| | GATTTCCAAGTCTCCACCCCATTGACGTCAATGGGAGTTTGT | |
| | TTTGGCACCAAAATCAACGGGACTTTCCAAAATGTCGTAACA | |
| | ACTCCGCCCCATTGACGCAAATGGGCGGTAGGCGTGTACGGT | |
| | GGGAGGTCTATATAAGCAGAGCTGGTTTAGTGAACCGT | |
| Cytomegalovirus | CGTTACATAACTTACGGTAAATGGCCCGCCTGGCTGACCGCC | 41 |
| (CMV) (third | CAACGACCCCCGCCCATTGACGTCAATAATGACGTATGTTCC | |
| version) | CATAGTAACGCCAATAGGGACTTTCCATTGACGTCAATGGGT | |
| | GGAGTATTTACGGTAAACTGCCCACTTGGCAGTACATCAAGT | |
| | GTATCATATGCCAAGTACGCCCCCTATTGACGTCAATGACGG | |
| | TAAATGGCCCGCCTGGCATTATGCCCAGTACATGACCTTATG | |
| | GGACTTTCCTACTTGGCAGTACATCTACGTATTAGTCATCGCT | |
| | ATTACCATGGTGATGCGGTTTTGGCAGTACATCAATGGGCGT | |
| | GGATAGCGGTTTGACTCACGGGGATTTCCAAGTCTCCACCCC | |
| | ATTGACGTCAATGGGAGTTTGTTTTGGCACCAAAATCAACGG | |
| | GACTTTCCAAAATGTCGTAACAACTCCGCCCCATTGACGCAA | |
| | ATGGGCGGTAGGCGTGTACGGTGGGAGGTCTATATAAGCAG | |
| | AGCT | |
| CAG promoter | ACTTACGGTAAATGGCCCGCCTGGCTGACCGCCCAACGACCC | 42 |
| (first version) | CCGCCCATTGACGTCAATAATGACGTATGTTCCCATAGTAAC | |
| | GCCAATAGGGACTTTCCATTGACGTCAATGGGTGGAGTATTT | |
| | ACGGTAAACTGCCCACTTGGCAGTACATCAAGTGTATCATAT | |
| | GCCAAGTACGCCCCCTATTGACGTCAATGACGGTAAATGGCC | |
| | CGCCTGGCATTATGCCCAGTACATGACCTTATGGGACTTTCC | |
| | TACTTGGCAGTACATCTACGTATTAGTCATCGCTATTACCAT | |
| | GGTCGAGGTGAGCCCCACGTTCTGCTTCACTCTCCCCATCTC | |
| | CCCCCCCTCCCCACCCCCAATTTTGTATTTATTTATTTTTTAAT | |
| | TATTTTGTGCAGCGATGGGGGCGGGGGGGGGGGGGGCGCGC | |
| | GCCAGGCGGGGCGGGGCGGGGCGAGGGGCGGGGCGGGGCG | |
| | AGGCGGAGAGGTGCGGCGGCAGCCAATCAGAGCGGCGCGCT | |
| | CCGAAAGTTTCCTTTTATGGCGAGGCGGCGGCGGCGGCGGCC | |
| | CTATAAAAAGCGAAGCGCGCGGCGGGCGG | |
| CAG promoter | CGTTACATAACTTACGGTAAATGGCCCGCCTGGCTGACCGCC | 43 |
| (second version) | CAACGACCCCCGCCCATTGACGTCAATAATGACGTATGTTCC | |
| | CATAGTAACGCCAATAGGGACTTTCCATTGACGTCAATGGGT | |
| | GGAGTATTTACGGTAAACTGCCCACTTGGCAGTACATCAAGT | |
| | GTATCATATGCCAAGTACGCCCCCTATTGACGTCAATGACGG | |
| | TAAATGGCCCGCCTGGCATTATGCCCAGTACATGACCTTATG | |
| | GGACTTTCCTACTTGGCAGTACATCTACGTATTAGTCATCGCT | |
| | ATTACCATGTCGAGGTGAGCCCCACGTTCTGCTTCACTCTCC | |
| | CCATCTCCCCCCCCTCCCCACCCCCAATTTTGTATTTATTTAT | |
| | TTTTTAATTATTTTGTGCAGCGATGGGGGCGGGGGGGGGGGG | |
| | GGCGCGCGCCAGGCGGGGCGGGGCGGGGCGAGGGGCGGGG | |
| | CGGGGCGAGGCGGAGAGGTGCGGCGGCAGCCAATCAGAGC | |
| | GGCGCGCTCCGAAAGTTTCCTTTTATGGCGAGGCGGCGGCGG | |
| | CGGCGGCCCTATAAAAAGCGAAGCGCGCGGCGGGCG | |
| Human EF1- | CAACCTTTGGAGCTAAGCCAGCAATGGTAGAGGGAAGATTC | 44 |
| alpha (EF1-α) | TGCACGTCCCTTCCAGGCGGCCTCCCCGTCACCACCCCCCCC | |
| | AACCCGCCCCGACCGGAGCTGAGAGTAATTCATACAAAAGG | |
| | ACTCGCCCCTGCCTTGGGGAATCCCAGGGACCGTCGTTAAAC | |
| | TCCCACTAACGTAGAACCCAGAGATCGCTGCGTTCCCGCCCC | |
| | CTCACCCGCCCGCTCTCGTCATCACTGAGGTGGAGAATAGCA | |
| | TGCGTGAGGCTCCGGTGCCCGTCAGTGGGCAGAGCGCACAT | |
| | CGCCCACAGTCCCCGAGAAGTTGGGGGGAGGGGTCGGCAAT | |
| | TGAACGGGTGCCTAGAGAAGGTGGCGCGGGGTAAACTGGGA | |
| | AAGTGATGTCGTGTACTGGCTCCGCCTTTTTCCCGAGGGTGG | |
| | GGGAGAACCGTATATAAGTGCAGTAGTCGCCGTGAACGTT | |
| Human | ACTTGTGGACAAAGTTTGCTCTATTCCACCTCCTCCAGGCCCT | 48 |
| CamKIIa | CCTTGGGTCCATCACCCCAGGGGTGCTGGGTCCATCCCACCC | |
| (CaMKIIa) | CCAGGCCCACACAGGCTTGCAGTATTGTGTGCGGTATGGTCA | |
| | GGGCGTCCGAGAGCAGGTTTCGCAGTGGAAGGCAGGCAGGT | |
| | GTTGGGGAGGCAGTTACCGGGGCAACGGGAACAGGGCGTTT | |
| | TGGAGGTGGTTGCCATGGGGACCTGGATGCTGACGAAGGCT | |
| | CGCGAGGCTGTGAGCAGCCACAGTGCCCTGC | |
| HSP70 | CCTGCAGGGCCGCCCACTCCCCCTTCCTCTCAGGGTCCCTGT | 115 |
| | CCCCTCCAGTGAATCCCAGAAGACTCTGGAGAGTTCTGAGCA | |
| | GGGGGCGGCACTCTGGCCTCTGATTGGTCCAAGGAAGGCTG | |
| | GGGGGCAGGACGGGAGGCGAAAACCCTGGAATATTCCCGAC | |
| | CTGGCAGCCTCATCGAGCTCGGTGATTGGCTCAGAAGGGAA | |
| | AAGGCGGGTCTCCGTGACGACTTATAAAAGCCCAGGGGCAA | |
| | GCGGTCCGGATAACGGCTAGCCTGAGGAGCTGCTGCGACAG | |
| | TCCACTACCTTTTTCGAGAGTGACTCCCGTTGTCCCAAGGCTT | |
| | CCCAGAGCGAACCTGTGCGGCTGCAGGCACCGGCGCGTCGA | |
| | GTTTCCGGCGTCCGGAAGGACCGAGCTCTTCTCGCGGATCCA | |
| | GTGTTCCGTTTCCAGCCCCCAATCTCAGAGCGGAGCCGACAG | |
| | AGAGCAGGGAACCC | |
| UBC | GATCTGGCCTCCGCGCCGGGTTTTGGCGCCTCCCGCGGGCGCCCC | 116 |
| | CCTCCTCACGGCGAGCGCTGCCACGTCAGACGAAGGGCGCAGGAG | |
| | CGTCCTGATCCTTCCGCCCGGACGCTCAGGACAGCGGCCCGCTGC | |
| | TCATAAGACTCGGCCTTAGAACCCCAGTATCAGCAGAAGGACATT | |
| | TTAGGACGGGACTTGGGTGACTCTAGGGCACTGGTTTTCTTTCCA | |
| | GAGAGCGGAACAGGCGAGGAAAAGTAGTCCCTTCTCGGCGATTCT | |
| | GCGGAGGGATCTCCGTGGGGCGGTGAACGCCGATGATTATATAAG | |
| | GACGCGCCGGGTGTGGCACAGCTAGTTCCGTCGCAGCCGGGATTT | |
| | GGGTCGCGGTTCTTGTTTGTGGATCGCTGTGATCGTCACTTGGTG | |
| | AGTAGCGGGCTGCTGGGCTGGCCGGGGCTTTCGTGGCCGCCGGGC | |
| | CGCTCGGTGGGACGGAAGCGTGTGGAGAGACCGCCAAGGGCTGTA | |
| | GTCTGGGTCCGCGAGCAAGGTTGCCCTGAACTGGGGGTTGGGGGG | |
| | AGCGCAGCAAAATGGCGGCTGTTCCCGAGTCTTGAATGGAAGACG | |
| | CTTGTGAGGCGGGCTGTGAGGTCGTTGAAACAAGGTGGGGGGCAT | |
| | GGTGGGCGGCAAGAACCCAAGGTCTTGAGGCCTTCGCTAATGCGG | |
| | GAAAGCTCTTATTCGGGTGAGATGGGCTGGGGCACCATCTGGGGA | |
| | CCCTGACGTGAAGTTTGTCACTGACTGGAGAACTCGGTTTGTCGT | |
| | CTGTTGCGGGGGCGGCAGTTATGCGGTGCCGTTGGGCAGTGCACC | |
| | CGTACCTTTGGGAGCGCGCGCCCTCGTCGTGTCGTGACGTCACCC | |
| | GTTCTGTTGGCTTATAATGCAGGGTGGGGCCACCTGCCGGTAGGT | |
| | GTGCGGTAGGCTTTTCTCCGTCGCAGGACGCAGGGTTCGGGCCTA | |
| | GGGTAGGCTCTCCTGAATCGACAGGCGCCGGACCTCTGGTGAGGG | |
| | GAGGGATAAGTGAGGCGTCAGTTTCTTTGGTCGGTTTTATGTACC | |
| | TATCTTCTTAAGTAGCTGAAGCTCCGGTTTTGAACTATGCGCTCG | |
| | GGGTTGGCGAGTGTGTTTTGTGAAGTTTTTTAGGCACCTTTTGAA | |
| | ATGTAATCATTTGGGTCAATATGTAATTTTCAGTGTTAGACTAGT | |
| | AAATTGTCCGCTAAATTCTGGCCGTTTTTGGCTTTTTTGTTAGAC | |
| | GAAG | |

In a certain embodiment, the vector genome comprises a polynucleotide sequence at least 75%, 80%, 85%, 90%, 92%, 93%, 94%, 95%, 96%, 97%, 98%, 99% or 11% identical to SEQ ID NO: 31. In a certain embodiment, the vector genome comprises a polynucleotide sequence at least 75%, 80%, 85%, 90%, 92%, 93%, 94%, 95%, 96%, 97%, 98%, 99%, or 100% identical to SEQ TD NO: 32. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 33. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 34. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 35. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 36. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 37. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 38. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 39. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 40. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 41. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 42. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 43. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 44. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 48. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 115. In a certain embodiment, the vector genome comprises a polynucleotide sequence at least 75%, 80%, 85%, 90%, 92%, 93%, 94%, 95%, 96%, 97%, 98%, 99%, or 100% identical to SEQ ID NO:116.

Further illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. A large variety of other promoters are known and generally available in the art, and the sequences of many such promoters are available in sequence databases such as the GenBank database.

In some cases, vectors of the present disclosure further comprise one or more regulatory elements selected from the group consisting of an enhancer, an intron, a poly-A signal, a 2A peptide encoding sequence, a WPRE (Woodchuck hepatitis virus posttranscriptional regulatory element), and a HPRE (Hepatitis B posttranscriptional regulatory element). In certain embodiments, the vectors comprise a mutant or modified WPRE (collectively referred to as WPRE(x), e.g., a WPRE or mutant or modified WPRE disclosed in any of SEQ ID NOs: 62-67 or 70, optionally the modified WPRE disclosed in SEQ ID NO: 63 (WPRE Mut6; see Zanta-Boussif, M., Charrier, S., Brice-Ouzet, A. et al. Validation of a mutated PRE sequence allowing high and sustained transgene expression while abrogating WHV-X protein synthesis: application to the gene therapy of WAS. Gene Ther 16, 605-619 (2009). https://doi.org/10.1038/gt.2009.3). In particular embodiments, the modified WPRE comprises a modification of the start codon of the WHV-X protein, thus preventing its expression.

In some embodiments, the vector comprises a CMV enhancer.

In certain embodiments, the vectors comprise one or more enhancers. In particular embodiments, the enhancer is a CMV enhancer sequence, a GAPDH enhancer sequence, a β-actin enhancer sequence, or an EF1-α enhancer sequence. Sequences of the foregoing are known in the art. For example, the sequence of the CMV immediate early (IE) enhancer is SEQ ID NO: 50.

| SEQ ID NO: 50 | |
|---|---|
| acttacggta aatggcccgc ctggctgacc gcccaacgac ccccgcccat tgacgtcaat | 60 |
| aatgacgtat gttcccatag taacgccaat agggactttc cattgacgtc aatgggtgga | 120 |
| gtatttacgg taaactgccc acttggcagt acatcaagtg tatcatatgc caagtacgcc | 180 |
| ccctattgac gtcaatgacg gtaaatggcc cgcctggcat tatgcccagt acatgacctt | 240 |
| atgggacttt cctacttggc agtacatcta cgtattagtc atcgctatta cca | 293 |

In certain embodiments, the vectors comprise one or more introns. In particular embodiments, the intron is a rabbit globin intron sequence, a chicken β-actin intron sequence, a synthetic intron sequence, an SV40 intron, or an EF1-α intron sequence.

In certain embodiments, the vectors comprise a polyA sequence. In particular embodiments, the polyA sequence is a rabbit globin polyA sequence, a human growth hormone polyA sequence, a bovine growth hormone polyA sequence, a PGK polyA sequence, an SV40 polyA sequence, or a TK polyA sequence. In some embodiments, the poly-A signal may be a bovine growth hormone polyadenylation signal (bGHpA).

In certain embodiments, the vectors comprise one or more transcript stabilizing element. In particular embodiments, the transcript stabilizing element is a WPRE sequence, a HPRE sequence, a scaffold-attachment region, a 3′ UTR, or a 5′ UTR. In particular embodiments, the vectors comprise both a 5′ UTR and a 3′ UTR.

In some embodiments, the vector comprises a 5′ untranslated region (UTR) selected from Table 2. In some embodiments, the vector genome comprises a polynucleotide sequence At least 75%, 80%, 85%, 90%, 92%, 93%, 94%, 95%, 96%, 97%, 98%, 99% or 100% identical to any one of SEQ TD NOS 51-61.

TABLE 2

| 5′ | | SEQ |
|---|---|---|
| UNTRANSLATED | | ID |
| REGION | SEQUENCE | NO: |
| Human beta-actin | CGCGTCCGCCCGCGAGCACAGAGCCTCGCCTTTGCCGATC | 51 |
| exon/intron | CGCCGCCCGTCCACACCCGCCGCCAGGTAAGCCCGGCCAG | |
| | CCGACCGGGGCATGCGGCCGCGGCCCTTCGCCCGTGCAGA | |
| | GCCGCCGTCTGGGCCGCAGCGGGGGGCGCATGGGGCGGA | |
| | ACCGGACCGCCGTGGGGGGCGCGGGAGAAGCCCCTGGGC | |
| | CTCCGGAGATGGGGGACACCCCACGCCAGTTCGCAGGCG | |
| | CGAGGCCGCGCTCGGGCGGGCGCGCTCCGGGGGTGCCGC | |
| | TCTCGGGGCGGGGGCAACCGGCGGGGTCTTTGTCTGAGCC | |
| | GGGCTCTTGCCAATGGGGATCGCACGGTGGGCGCGGCGTA | |
| | GCCCCCGTCAGGCCCGGTGGGGGCTGGGGCGCCATGCGC | |
| | GTGCGCGCTGGTCCTTTGGGCGCTAACTGCGTGCGCGCTG | |
| | GGAATTGGCGCTAATTGCGCGTGCGCGCTGGGACTCAATG | |
| | GCGCTAATCGCGCGTGCGTTCTGGGGCCCGGGCGCTTGCG | |
| | CCACTTCCTGCCCGAGCCGCTGGCGCCCGAGGGTGTGGCC | |
| | GCTGCGTGCGCGCGCGCGACCCGGTCGCTGTTTGAACCGG | |
| | GCGGAGGCGGGGCTGGCGCCCGGTTGGGAGGGGGTTGGG | |
| | GCCTGGCTTCCTGCCGCGCGCCGCGGGGACGCCTCCGACC | |
| | AGTGTTTGCCTTTTATGGTAATAACGCGGCCGGCCCGGCT | |
| | TCCTTTGTCCCCAATCTGGGCGCGCGCCGGCGCCCCCTGG | |
| | CGGCCTAAGGACTCGGCGCGCCGGAAGTGGCCAGGGCGG | |
| | CAGCGGCTGCTCTTGGCGGCCCCGAGGTGACTATAGCCTT | |
| | CTTTTGTGTCTTGATAGTTCGCCAGCCTCTGCTAACCATGT | |
| | TCATGCCTTCTTCTTTTTCCTACAGCTCCTGGGCAACGTGC | |
| | TGGTTATTGTGCTGTCTCATCATTTTGGCAAAGAATTC | |
| Chicken beta-actin | GTCGCTGCGCGCTGCCTTCGCCCCGTGCCCCGCTCCGCCG | 52 |
| exon/intron + rabbit | CCGCCTCGCGCCGCCCGCCCCGGCTCTGACTGACCGCGTT | |
| globin intron | ACTCCCACAGGTGAGCGGGCGGGACGGCCCTTCTCCTCCG | |
| | GGCTGTAATTAGCGCTTGGTTTAATGACGGCTTGTTTCTTT | |
| | TCTGTGGCTGCGTGAAAGCCTTGAGGGGCTCCGGGAGGGC | |
| | CCTTTGTGCGGGGGGAGCGGCTCGGGGGGTGCGTGCGTGT | |
| | GTGTGTGCGTGGGGAGCGCCGCGTGCGGCTCCGCGCTGCC | |
| | CGGCGGCTGTGAGCGCTGCGGGCGCGGCGCGGGGCTTTGT | |
| | GCGCTCCGCAGTGTGCGCGAGGGGAGCGCGGCCGGGGGC | |
| | GGTGCCCCGCGGTGCGGGGGGGGCTGCGAGGGGAACAAA | |
| | GGCTGCGTGCGGGGTGTGTGCGTGGGGGGGTGAGCAGGG | |
| | GGTGTGGGCGCGTCGGTCGGGCTGCAACCCCCCCTGCACC | |
| | CCCCTCCCCGAGTTGCTGAGCACGGCCCGGCTTCGGGTGC | |
| | GGGGCTCCGTACGGGGCGTGGCGCGGGGCTCGCCGTGCC | |
| | GGGCGGGGGGTGGCGGCAGGTGGGGGTGCCGGGCGGGGC | |
| | GGGGCCGCCTCGGGCCGGGGAGGGCTCGGGGGAGGGGCG | |
| | CGGCGGCCCCCGGAGCGCCGGCGGCTGTCGAGGCGCGGC | |
| | GAGCCGCAGCCATTGCCTTTTATGGTAATCGTGCGAGAGG | |
| | GCGCAGGGACTTCCTTTGTCCCAAATCTGTGCGGAGCCGA | |
| | AATCTGGGAGGCGCCGCCGCACCCCCTCTAGCGGGCGCGG | |
| | GGCGAAGCGGTGCGGCGCCGGCAGGAAGGAAATGGGCGG | |
| | GGAGGGCCTTCGTGCGTCGCCGCGCCGCCGTCCCCTTCTC | |
| | CCTCTCCAGCCTCGGGGCTGTCCGCGGGGGGACGGCTGCC | |
| | TTCGGGGGGGACGGGGCAGGGCGGGGTTCGGCTTCTGGC | |
| | GTGTGACCGGCGGCTCTAGAGCCTCTGCTAACCATGTTCA | |
| | TGCCTTCTTCTTTTTCCTACAGCTCCTGGGCAACGTGCTGG | |
| | TTATTGTGCTGTCTCATCATTTTGGCAAAGAATTC | |
| SV40 intron | GGTAAGTTTAGTCTTTTTGTCTTTTATTTCAGGTCCCGGAT | 53 |
| (Chimeric intron | CCGGTGGTGGTGCAAATCAAAGAACTGCTCCTCAGTGGAT | |
| sequence) | GTTGCCTTTACTTCTAGGCCTGTACGGAAGTGTTACTTCTG | |
| | CTCTAAAAGCTGCGGAATTGTACCCGC | |
| 5′ UTR-Syn1 Hs | AGTCTGCGGTGGGCAGCGGAGGAGTCGTGTCGTGCCTGAG | 54 |
| | AGCGCAGCTGTGCTCCTGGGCACCGCGCAGTCCGCCCCCG | |
| | CGGCTCCTGGCCAGACCACCCCTAGGACCCCCTGCCCCAA | |
| | GTCGCA | |
| CMV IE exon | TCAGATCGCCTGGAGAGGCCATCCACGCTGTTTTGACCTC | 55 |
| | CATAGTGGACACCGGGACCGATCCAGCCTCCGCGGCCGG | |
| | GAACGGTGCATTGGAACGCGGATTCCCCGTGCCAAGAGTG | |
| | AC | |
| TPL-eBAG3 | CTCACTCTCTTCCGCATCGCTGTCTGCGAGGGCCAGCTGTT | 56 |
| (adenovirus derived | GGGCTCGCGGTTGAGGACAAACTCTTCGCGGTCTTTCCAG | |
| enhancer element) | TACTCTTGGATCGGAAACCCGTCGGCCTCCGAACGGTACT | |
| | CCGCCACCGAGGGACCTGAGCGAGTCCGCATCGACCGGA | |
| | TCGGAAAACCTCTCGAGAAAGGCGTCTAACCAGTCACAGT | |
| | CGCAAGGTAGGCTGAGCACCGTGGCGGGCGGCAGCGGGT | |
| | GGCGGTCGGGGTTGTTTCTGGCGGAGGTGCTGCTGATGAT | |
| | GTAATTAAAGTAGGCGGTCTTGAGACGGCGGATGGTCGA | |
| | GGTGAGGTGTGGCAGGCTTGAGATCCAGCTGTTGGGGTGA | |
| | GTACTCCCTCTCAAAAGCGGGCATTACTTCTGCGCTAAGA | |
| | TTGTCAGTTTCCAAAAACGAGGAGGATTTGATATTCACCT | |
| | GGCCCGATCTGGCCATACACTTGAGTGACAATGACATCCA | |
| | CTTTGCCTTTCTCTCCACAGGTGTCCACTCCCAG | |
| Human EF1-α | CTTTTTCGCAACGGGTTTGCCGCCAGAACACAGGTAAGTG | 57 |
| intron/exon | CCGTGTGTGGTTCCCGCGGGCCTGGCCTCTTTACGGGTTAT | |
| | GGCCCTTGCGTGCCTTGAATTACTTCCACCTGGCTCCAGTA | |
| | CGTGATTCTTGATCCCGAGCTGGAGCCAGGGGCGGGCCTT | |
| | GCGCTTTAGGAGCCCCTTCGCCTCGTGCTTGAGTTGAGGC | |
| | CTGGCCTGGGCGCTGGGGCCGCCGCGTGCGAATCTGGTGG | |
| | CACCTTCGCGCCTGTCTCGCTGCTTTCGATAAGTCTCTAGC | |
| | CATTTAAAATTTTTGATGACGTGCTGCGACGCTTTTTTTCT | |
| | GGCAAGATAGTCTTGTAAATGCGGGCCAGGATCTGCACAC | |
| | TGGTATTTCGGTTTTTGGGCCCGCGGCCGGCGACGGGGCC | |
| | CGTGCGTCCCAGCGCACATGTTCGGCGAGGCGGGGCCTGC | |
| | GAGCGCGGCCACCGAGAATCGGACGGGGGTAGTCTCAAG | |
| | CTGGCCGGCCTGCTCTGGTGCCTGGCCTCGCGCCGCCGTG | |
| | TATCGCCCCGCCCTGGGCGGCAAGGCTGGCCCGGTCGGCA | |
| | CCAGTTGCGTGAGCGGAAAGATGGCCGCTTCCCGGCCCTG | |
| | CTCCAGGGGGCTCAAAATGGAGGACGCGGCGCTCGGGAG | |
| | AGCGGGCGGGTGAGTCACCCACACAAAGGAAAAGGGCCT | |
| | TTCCGTCCTCAGCCGTCGCTTCATGTGACTCCACGGAGTAC | |
| | CGGGCGCCGTCCAGGCACCTCGATTAGTTCTGGAGCTTTT | |
| | GGAGTACGTCGTCTTTAGGTTGGGGGGAGGGGTTTTATGC | |
| | GATGGAGTTTCCCCACACTGAGTGGGTGGAGACTGAAGTT | |
| | AGGCCAGCTTGGCACTTGATGTAATTCTCCTTGGAATTTG | |
| | GCCTTTTTGAGTTTGGATCTTGGTTCATTCTCAAGCCTCAG | |
| | ACAGTGGTTCAAAGTTTTTTTCTTCCATTTCAG | |
| Human EF1-α, | GTAAGTGCCGTGTGTGGTTCCCGCGGGCCTGGCCTCTTTA | 58 |
| intron A | CGGGTTATGGCCCTTGCGTGCCTTGAATTACTTCCACCTGG | |
| | CTGCAGTACGTGATTCTTGATCCCGAGCTTCGGGTTGGAA | |
| | GTGGGTGGGAGAGTTCGAGGCCTTGCGCTTAAGGAGCCCC | |
| | TTCGCCTCGTGCTTGAGTTGAGGCCTGGCCTGGGCGCTGG | |
| | GGCCGCCGCGTGCGAATCTGGTGGCACCTTCGCGCCTGTC | |
| | TCGCTGCTTTCGATAAGTCTCTAGCCATTTAAAATTTTTGA | |
| | TGACCTGCTGCGACGCTTTTTTTCTGGCAAGATAGTCTTGT | |
| | AAATGCGGGCCAAGATCTGCACACTGGTATTTCGGTTTTT | |
| | GGGGCCGCGGGCGGCGACGGGGCCCGTGCGTCCCAGCGC | |
| | ACATGTTCGGCGAGGCGGGGCCTGCGAGCGCGGCCACCG | |
| | AGAATCGGACGGGGGTAGTCTCAAGCTGGCCGGCCTGCTC | |
| | TGGTGCCTGGCCTCGCGCCGCCGTGTATCGCCCCGCCCTG | |
| | GGCGGCAAGGCTGGCCCGGTCGGCACCAGTTGCGTGAGC | |
| | GGAAAGATGGCCGCTTCCCGGCCCTGCTGCAGGGAGCTCA | |
| | AAATGGAGGACGCGGCGCTCGGGAGAGCGGGCGGGTGAG | |
| | TCACCCACACAAAGGAAAAGGGCCTTTCCGTCCTCAGCCG | |
| | TCGCTTCATGTGACTCCACGGAGTACCGGGCGCCGTCCAG | |
| | GCACCTCGATTAGTTCTCGAGCTTTTGGAGTACGTCGTCTT | |
| | TAGGTTGGGGGGAGGGGTTTTATGCGATGGAGTTTCCCCA | |
| | CACTGAGTGGGTGGAGACTGAAGTTAGGCCAGCTTGGCAC | |
| | TTGATGTAATTCTCCTTGGAATTTGCCCTTTTTGAGTTTGG | |
| | ATCTTGGTTCATTCTCAAGCCTCAGACAGTGGTTCAAAGTT | |
| | TTTTTCTTCCATTTCAG | |
| 5′ UTR human | TCAGAAGCCCCGGGCTCGTCAGTCAAACCGGTTCTCTGTT | 59 |
| CamKIIa | TGCACTCGGCAGCACGGGCAGGCAAGTGGTCCCTAGGTTC | |
| | GGG | |
| B-globin intron | GTGAGTCTATGGGACCCTTGATGTTTTCTTTCCCCTTCTTTT | 60 |
| | CTATGGTTAAGTTCATGTCATAGGAAGGGGAGAAGTAACA | |
| | GGGTACACATATTGACCAAATCAGGGTAATTTTGCATTTG | |
| | TAATTTTAAAAAATGCTTTCTTCTTTTAATATACTTTTTTGT | |
| | TTATCTTATTTCTAATACTTTCCCTAATCTCTTTCTTTCAGG | |
| | GCAATAATGATACAATGTATCATGCCTCTTTGCACCATTCT | |
| | AAAGAATAACAGTGATAATTTCTGGGTTAAGGCAATAGCA | |
| | ATATTTCTGCATATAAATATTTCTGCATATAAATTGTAACT | |
| | GATGTAAGAGGTTTCATATTGCTAATAGCAGCTACAATCC | |
| | AGCTACCATTCTGCTTTTATTTTATGGTTGGGATAAGGCTG | |
| | GATTATTCTGAGTCCAAGCTAGGCCCTTTTGCTAATCATGT | |
| | TCATACCTCTTATCTTCCTCCCACAG | |
| SV40 intron (long | TCTAGAGGATCCGGTACTCGAGGAACTGAAAAACCAGAA | 61 |
| form; underlined 5′ | AGTTAACTGGTAAGTTTAGTCTTTTTGTCTTTTATTTCAGG | |
| and 3′ extensions) | TCCCGGATCCGGTGGTGGTGCAAATCAAAGAACTGCTCCT | |
| | CAGTGGATGTTGCCTTTACTTCTAGGCCTGTACGGAAGTG | |
| | TTACTTCTGCTCTAAAAGCTGCGGAATTGTACCCGC | |

In some embodiments, the vector comprises a 3′ untranslated region selected from Table 3. In some embodiments, the vector genome comprises a polynucleotide sequence at least 75%, 80%, 85%, 90%, 92%, 93%, 94%, 95%, 96%, 97%, 98%, 99% or 100 identical to any one of SEQ TD NOS 62-70.

TABLE 3

| | | SEQ |
|---|---|---|
| 3′ UNTRANSLATED | | ID |
| REGION | SEQUENCE | NO: |
| WPRE(x) (mutated | AATCAACCTCTGGATTACAAAATTTGTGAAAGATTGACT | 62 |
| woodchuck hepatitis | GGTATTCTTAACTATGTTGCTCCTTTTACGCTATGTGGAT | |
| regulatory element- | ACGCTGCTTTAATGCCTTTGTATCATGCTATTGCTTCCCGT | |
| version 1) | ATGGCTTTCATTTTCTCCTCCTTGTATAAATCCTGGTTGCT | |
| | GTCTCTTTATGAGGAGTTGTGGCCCGTTGTCAGGCAACGT | |
| | GGCGTGGTGTGCACTGTGTTTGCTGACGCAACCCCCACTG | |
| | GTTGGGGCATTGCCACCACCTGTCAGCTCCTTTCCGGGAC | |
| | TTTCGCTTTCCCCCTCCCTATTGCCACGGCGGAACTCATC | |
| | GCCGCCTGCCTTGCCCGCTGCTGGACAGGGGCTCGGCTGT | |
| | TGGGCACTGACAATTCCGTGGTGTTGTCGGGGAAATCAT | |
| | CGTCCTTTCCTTGGCTGCTCGCCTGTGTTGCCACCTGGATT | |
| | CTGCGCGGGACGTCCTTCTGCTACGTCCCTTCGGCCCTCA | |
| | ATCCAGCGGACCTTCCTTCCCGCGGCCTGCTGCCGGCTCT | |
| | GCGGCCTCTTCCGCGTCTTCGCCTTCGCCCTCAGACGAGT | |
| | CGGATCTCCCTTTGGGCCGCCTCCCCGC | |
| WPRE(x) (mutated | TCAACCTCTGGATTACAAAATTTGTGAAAGATTGACTGGT | 63 |
| woodchuck hepatitis | ATTCTTAACTATGTTGCTCCTTTTACGCTATGTGGATACG | |
| regulatory element- | CTGCTTTAATGCCTTTGTATCATGCTATTGCTTCCCGTATG | |
| version 2; WPRE | GCTTTCATTTTCTCCTCCTTGTATAAATCCTGGTTGCTGTC | |
| Mut6) | TCTTTATGAGGAGTTGTGGCCCGTTGTCAGGCAACGTGGC | |
| | GTGGTGTGCACTGTGTTTGCTGACGCAACCCCCACTGGTT | |
| | GGGGCATTGCCACCACCTGTCAGCTCCTTTCCGGGACTTT | |
| | CGCTTTCCCCCTCCCTATTGCCACGGCGGAACTCATCGCC | |
| | GCCTGCCTTGCCCGCTGCTGGACAGGGGCTCGGCTGTTGG | |
| | GCACTGACAATTCCGTGGTGTTGTCGGGGAAATCATCGTC | |
| | CTTTCCTTGGCTGCTCGCCTGTGTTGCCACCTGGATTCTGC | |
| | GCGGGACGTCCTTCTGCTACGTCCCTTCGGCCCTCAATCC | |
| | AGCGGACCTTCCTTCCCGCGGCCTGCTGCCGGCTCTGCGG | |
| | CCTCTTCCGCGTCTTCGCCTTCGCCCTCAGACGAGTCGGA | |
| | TCTCCCTTTGGGCCGCCTCCCCGCA | |
| WPRE(x) (mutated | TTCCTGTTAATCAACCTCTGGATTACAAAATTTGTGAAAG | 64 |
| woodchuck hepatitis | ATTGACTGGTATTCTTAACTATGTTGCTCCTTTTACGCTAT | |
| regulatory element- | GTGGATACGCTGCTTTAATGCCTTTGTATCATGCTATTGC | |
| version 3) | TTCCCGTATGGCTTTCATTTTCTCCTCCTTGTATAAATCCT | |
| | GGTTGCTGTCTCTTTATGAGGAGTTGTGGCCCGTTGTCAG | |
| | GCAACGTGGCGTGGTGTGCACTGTGTTTGCTGACGCAAC | |
| | CCCCACTGGTTGGGGCATTGCCACCACCTGTCAGCTCCTT | |
| | TCCGGGACTTTCGCTTTCCCCCTCCCTATTGCCACGGCGG | |
| | AACTCATCGCCGCCTGCCTTGCCCGCTGCTGGACAGGGG | |
| | CTCGGCTGTTGGGCACTGACAATTCCGTGGTGTTGTCGGG | |
| | GAAGCTGACGTCCTTTCCGCGGCTGCTCGCCTGTGTTGCC | |
| | ACCTGGATTCTGCGCGGGACGTCCTTCTGCTACGTCCCTT | |
| | CGGCCCTCAATCCAGCGGACCTTCCTTCCCGCGGCCTGCT | |
| | GCCGGCTCTGCGGCCTCTTCCGCCTCTTCGCCTTCGCCCT | |
| | CAGACGAGTCGGATCTCCCTTTGGGCCGCCTCCCCGCCCA | |
| | TGTATCTTTTTCACCTGTGCCTTGTTTTTGCCTGTGTTCCG | |
| | CGTCCTACTTTTCAAGCCTCCAAGCTGTGCCTTGGGCGGC | |
| | TTTGGGGCATGGACATAGATCCCTATAAAGAATTTGGTTC | |
| | ATCTTATCAGTTGTTGAATTTTCTTCCTTTGGAC | |
| CAAX | TGTGTGATAATG | 65 |
| EES | CTGTTCTCATCACATCATATCAAGGTTATATACCATCAAT | 66 |
| | ATTGCCACAGATGTTACTTAGCCTTTTAATATTTCTCTAAT | |
| | TTAGTGTATATGCAATGATAGTTCTCTGATTTCTGAGATT | |
| | GAGTTTCTCATGTGTAATGATTATTTAGAGTTTCTCTTTCA | |
| | TCTGTTCAAATTTTTGTCTAGTTTTATTTTTTACTGATTTG | |
| | TAAGACTTCTTTTTATAATCTGCATATTACAATTCTCTTTA | |
| | CTGGGGTGTTGCAAATATTTTCTGTCATTCTATGGCCTGA | |
| | CTTTTCTTAATGGTTTTTTAATTTTAAAAATAAGTCTTAAT | |
| | ATTCATGCAATCTAATTAACAATCTTTTCTTTGTGGTTAG | |
| | GACTTTGAGTCATAAGAAATTTTTCTCTACACTGAAGTCA | |
| | TGATGGCATGCTTCTATATTATTTTCTAAAAGATTTAAAG | |
| | TTTTGCCTTCTCCATTTAGACTTATAATTCACTGGAATTTT | |
| | TTTGTGTGTATGGTATGACATATGGGTTCCCTTTTATTTTT | |
| | TACATATAAATATATTTCCCTGTTTTTCTAAAAAAGAAAA | |
| | AGATCATCATTTTCCCATTGTAAAATGCCATATTTTTTTCA | |
| | TAGGTCACTTACATATATCAATGGGTCTGTTTCTGAGCTC | |
| | TACTCTATTTTATCAGCCTCACTGTCTATCCCCACACATCT | |
| | CATGCTTTGCTCTAAATCTTGATATTTAGTGGAACATTCT | |
| | TTCCCATTTTGTTCTACAAGAATATTTTTGTTATTGTCTTT | |
| | GGGCTTTCTATATACATTTTGAAATGAGGTTGACAAGTTA | |
| HPRE | ATAACAGGCCTATTGATTGGAAAGTTTGTCAACGAATTGT | 67 |
| | GGGTCTTTTGGGGTTTGCTGCCCCTTTTACGCAATGTGGA | |
| | TATCCTGCTTTAATGCCTTTATATGCATGTATACAAGCAA | |
| | AACAGGCTTTTACTTTCTCGCCAACTTACAAGGCCTTTCT | |
| | CAGTAAACAGTATATGACCCTTTACCCCGTTGCTCGGCAA | |
| | CGGCCTGGTCTGTGCCAAGTGTTTGCTGACGCAACCCCCA | |
| | CTGGTTGGGGCTTGGCCATAGGCCATCAGCGCATGCGTG | |
| | GAACCTTTGTGTCTCCTCTGCCGATCCATACTGCGGAACT | |
| | CCTAGCCGCTTGTTTTGCTCGCAGCAGGTCTGGAGCAAAC | |
| | CTCATCGGGACCGACAATTCTGTCGTACTCTCCCGCAAGT | |
| | ATACATCGTTTCCATGGCTGCTAGGCTGTGCTGCCAACTG | |
| | GATCCTGCGCGGGACGTCCTTTGTTTACGTCCCGTCGGCG | |
| | CTGAATCCCGCGGACGACCCCTCCCGGGGCCGCTTGGGG | |
| | CTCTACCGCCCGCTTCTCCGTCTGCCGTACCGTCCGACCA | |
| | CGGGGCGCACCTCTCTTTACGCGGACTCCCCGTCTGTGCC | |
| | TTCTCATCTGCCGGACCGTGTGCACTTCGCTTCACCTCTG | |
| | CACGTCGCATGGAGGCCACCGTGAACGCCCACCGGAACC | |
| | TGCCCAAGGTCTTGCATAAGAGGACTCTTGGACTTTCAGC | |
| | AATGTCATC | |
| R2V17 (HepB derived | TTCCTGTAAACAGGCCTATTGATTGGAAAGTTTGTCAACG | 68 |
| enhancer element) | AATTGTGGGTCTTTTGGGGTTTGCTGCCCCTTTTACGCAA | |
| | TGTGGATATCCTGCTTTAATGCCTTTATATGCATGTATAC | |
| | AAGCAAAACAGGCTTTTACTTTCTCGCCAACTTACAAGGC | |
| | CTTTCTCAGTAAACAGTATATGACCCTTTACCCCGTTGCT | |
| | CGGCAACGGCCTGGTCTGTGCCAAGTGTTTGCTGACGCA | |
| | ACCCCCACTGGTTGGGGCTTGGCCATAGGCCATCAGCGC | |
| | ATGCGTGGAACCTTTGTGTCTCCTCTGCCGATCCATACTG | |
| | CGGAACTCCTAGCCGCTTGTTTTGCTCGCAGCTGGACTGG | |
| | AGCAAACCTCATCGGGACCGACAATTCTGTCGTACTCTCC | |
| | CGCAAGCACTCACCGTTTCCGCGGCTGCTCGCCTGTGTTG | |
| | CCACCTGGATTCTGCGCGGGACGTCCTTCTGCTACGTCCC | |
| | TTCGGCCCTCAATCCAGCGGACCTTCCTTCCCGCGGCCTG | |
| | CTGCCGGCTCTGCGGCCTCTTCCGCCTCTTCGCCTTCGCC | |
| | CTCAGACGAGTCGGATCTCCCTTTGGGCCGCCTCCCCGCC | |
| | CATGTATCTTTTTCACCTGTGCCTTGTTTTTGCCTGTGTTC | |
| | CGCGTCCTACTTTTCAAGCCTCCAAGCTGTGCCTTGGGCG | |
| | GCTTTGGGGCATGGACATAGATCCCTATAAAGAATTTGG | |
| | TTCATCTTATCAGTTGTTGAATTTTCTTCCTTTGGAC | |
| 3′UTR(globin) | GCTGGAGCCTCGGTAGCCGTTCCTCCTGCCCGCTGGGCCT | 69 |
| | CCCAACGGGCCCTCCTCCCCTCCTTGCACCGGCCCTTCCT | |
| | GGTCTTTGAATAAA | |
| WPRE(r) | ATTCGAGCATCTTACCGCCATTTATTCCCATATTTGTTCTG | 70 |
| | TTTTTCTTGATTTGGGTATACATTTAAATGTTAATAAAAC | |
| | AAAATGGTGGGGCAATCATTTACATTTTTAGGGATATGTA | |
| | ATTACTAGTTCAGGTGTATTGCCACAAGACAAACATGTTA | |
| | AGAAACTTTCCCGTTATTTACGCTCTGTTCCTGTTAATCA | |
| | ACCTCTGGATTACAAAATTTGTGAAAGATTGACTGATATT | |
| | CTTAACTATGTTGCTCCTTTTACGCTGTGTGGATATGCTG | |
| | CTTTAATGCCTCTGTATCATGCTATTGCTTCCCGTACGGCT | |
| | TTCGTTTTCTCCTCCTTGTATAAATCCTGGTTGCTGTCTCT | |
| | TTATGAGGAGTTGTGGCCCGTTGTCCGTCAACGTGGCGTG | |
| | GTGTGCTCTGTGTTTGCTGACGCAACCCCCACTGGCTGGG | |
| | GCATTGCCACCACCTGTCAACTCCTTTCTGGGACTTTCGC | |
| | TTTCCCCCTCCCGATCGCCACGGCAGAACTCATCGCCGCC | |
| | TGCCTTGCCCGCTGCTGGACAGGGGCTAGGTTGCTGGGC | |
| | ACTGATAATTCCGTGGTGTTGTCGGGGAAGGGCC | |

In some embodiments, the vector comprises a polyadenylation (polyA) signal selected from Table 4. In some embodiments, the polyA signal comprises a polynucleotide sequence at least 75%, 80%, 85%, 90%, 92%, 93%, 94%, 95%, 96%, 97%, 98%, 99% or 100% identical to any one of SEQ ID NOS 71-75.

TABLE 4

| POLY- | | SEQ |
|---|---|---|
| ADENYLATION | | ID |
| SITE | SEQUENCE | NO: |
| Rabbit globin | TGGCTAATAAAGGAAATTTATTTTCATTGCAATAGTGTG | 71 |
| (pAGlobin-Oc) | TTGGAATTTTTTGTGTCTCTCACTCGGAAGAACATATGG | |
| | GAGGGCAAATCATTTAAAACATCAGAATGAGTATTTGGT | |
| | TTAGAGTTTGGCAACATATGCCCATATGCTGGCTGCCAT | |
| | GAACAAAGGTTGGCTATAAAGAGGTCATCAGTATATGA | |
| | AACAGCCCCCTGCTGTCCATTCCTTATTCCATAGAAAAG | |
| | CCTTGACTTGAGGTTAGATTTTTTTTATATTTTGTTTTGTG | |
| | TTATTTTTTTCTTTAACATCCCTAAAATTTTCCTTACATGT | |
| | TTTACTAGCCAGATTTTTCCTCCTCTCCTGACTACTCCCA | |
| | GTCATAGCTGTCCCTCTTCTCTTATGGAGATC | |
| Bovine growth | TTGCCAGCCATCTGTTGTTTGCCCCTCCCCCGTGCCTTCC | 72 |
| hormone (pAGH-Bt- | TTGACCCTGGAAGGTGCCACTCCCACTGTCCTTTCCTAA | |
| version 1) | TAAAATGAGGAAATTGCATCGCATTGTCTGAGTAGGTGT | |
| | CATTCTATTCTGGGGGGTGGGGTGGGGCAGGACAGCAA | |
| | GGGGGAGGATTGGGAATACAATAGCAGGCATGCTGGGG | |
| | ATGCGGTGGGCTCTATGGGTACCCAGGTGCTGAAGAATT | |
| | GACCCGGTTCCTCCTGGG | |
| Bovine growth | TTGCCAGCCATCTGTTGTTTGCCCCTCCCCCGTGCCTTCC | 73 |
| hormone (pAGH-Bt- | TTGACCCTGGAAGGTGCCACTCCCACTGTCCTTTCCTAA | |
| version 2) | TAAAATGAGGAAATTGCATCGCATTGTCTGAGTAGGTGT | |
| | CATTCTATTCTGGGGGGTGGGGTGGGGCAGGACAGCAA | |
| | GGGGGAGGATTGGGAAGACAATAGCAGGCATGCTGGGG | |
| | ATGCGGTGGGCTCTATGGGTACCCAGGTGCTGAAGAATT | |
| | GACCCGGTTCCTCCTGGG | |
| Bovine growth | CTGTGCCTTCTAGTTGCCAGCCATCTGTTGTTTGCCCCTC | 74 |
| hormone (pAGH-Bt- | CCCCGTGCCTTCCTTGACCCTGGAAGGTGCCACTCCCAC | |
| version 3) | TGTCCTTTCCTAATAAAATGAGGAAATTGCATCGCATTG | |
| | TCTGAGTAGGTGTCATTCTATTCTGGGGGGTGGGGTGGG | |
| | GCAGGACAGCAAGGGGGAGGATTGGGAAGACAATAGC | |
| | AGGCATGCTGGGGATGCGGTGGGCTCTATGG | |
| Human growth | CTGCCCGGGTGGCATCCCTGTGACCCCTCCCCAGTGCCT | 75 |
| hormone (pAGH-Hs) | CTCCTGGCCCTGGAAGTTGCCACTCCAGTGCCCACCAGC | |
| | CTTGTCCTAATAAAATTAAGTTGCATCATTTTGTCTGACT | |
| | AGGTGTCCTTCTATAATATTATGGGGTGGAGGGGGGTGG | |
| | TATGGAGCAAGGGGCCCAAGTTGGGAAGAAACCTGTAG | |
| | GGCCTGC | |

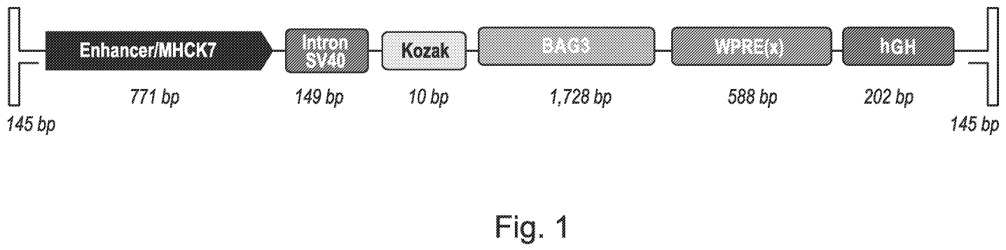

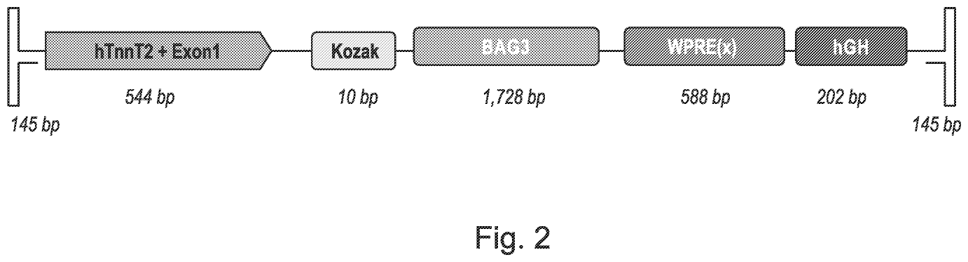

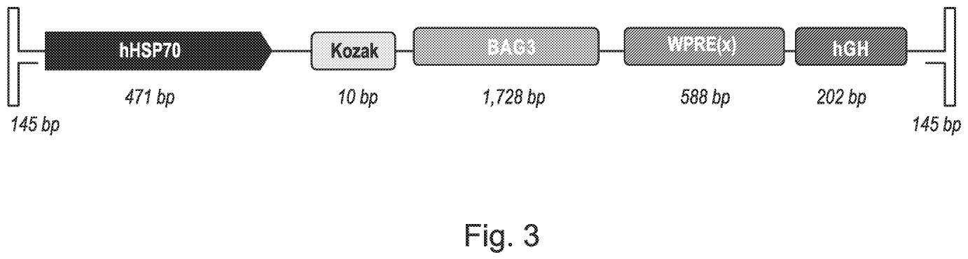

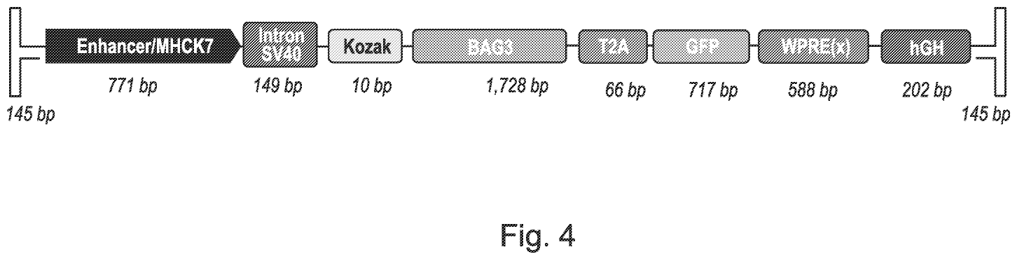

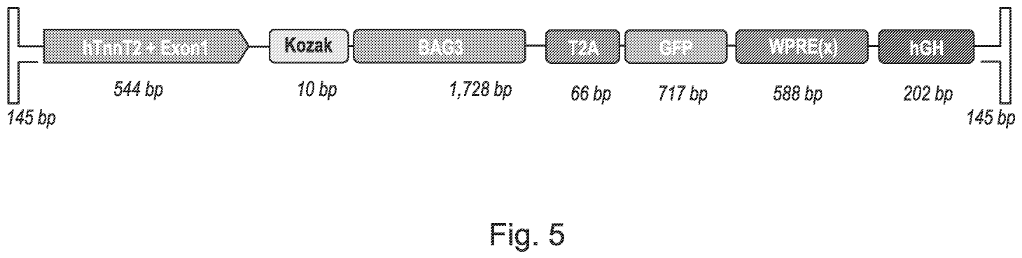

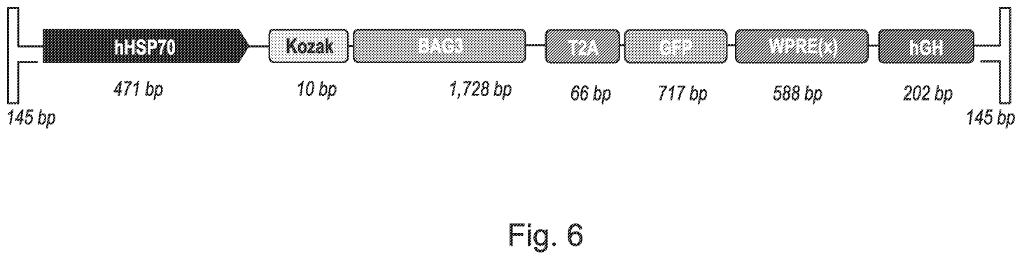

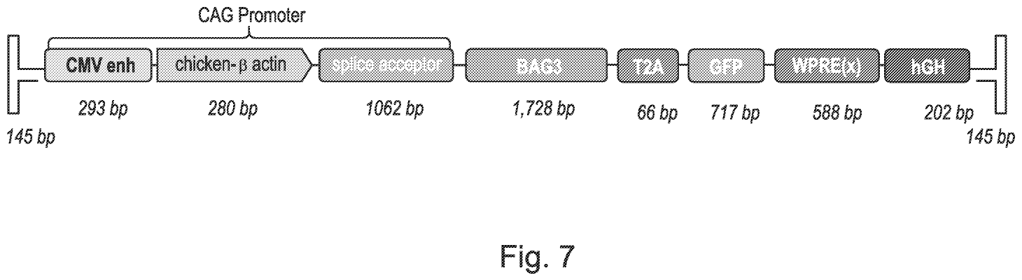

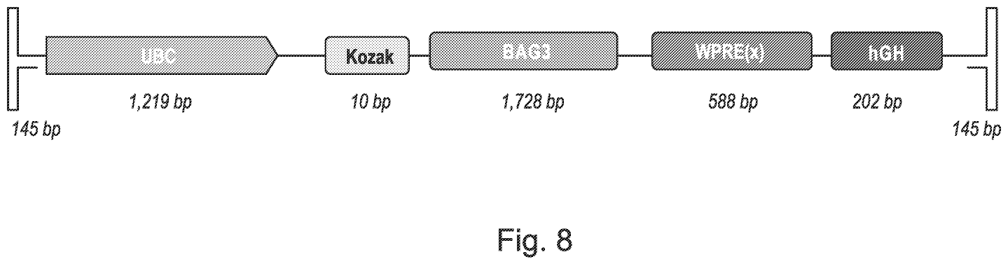

Illustrative vector genomes are depicted in FIGS. 1-8; and provided as SEQ ID NOs: 107-114. In some embodiments, the vector genome comprises, consists essentially of, or consists of a polynucleotide sequence that shares at least 90%, 91%, 92%, 93%, 94%, 95%, 96%, 97%, 98%, 99%, or 100% identity to any one of SEQ ID NOs: 26-30 and 76-95, optionally with or without the ITR sequences.

In a certain embodiment, the vector genome comprises, in 5′ to 3′ order, a 5′ ITR; an MHCK7 promoter; an SV-40 Chimeric Intron, a BAG3 transgene; an WPRE(x) element; a Human GH poly(A) signal (hGH) sequence; and a 3′ ITR. The vector genome may comprise, in 5′ to 3′ order, the polynucleotide sequences SEQ ID NO: 107; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. In certain embodiments, this vector genome is packaged in an AAVrh74 vector.

| SEQ ID NO: 107 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtac ccttcagatt aaaaataact | 180 |
| gaggtaaggg cctgggtagg ggaggtggtg tgagacgctc ctgtctctcc tctatctgcc | 240 |
| catcggccct ttggggagga ggaatgtgcc caaggactaa aaaaaggcca tggagccaga | 300 |
| ggggcgaggg caacagacct ttcatgggca aaccttgggg ccctgctgtc tagcatgccc | 360 |
| cactacgggt ctaggctgcc catgtaagga ggcaaggcct ggggacaccc gagatgcctg | 420 |
| gttataatta acccagacat gtggctgccc cccccccccc aacacctgct gcctctaaaa | 480 |
| ataaccctgt ccctggtgga tcccctgcat gcgaagatct tcgaacaagg ctgtggggga | 540 |
| ctgagggcag gctgtaacag gcttgggggc cagggcttat acgtgcctgg gactcccaaa | 600 |
| gtattactgt tccatgttcc cggcgaaggg ccagctgtcc cccgccagct agactcagca | 660 |
| cttagtttag gaaccagtga gcaagtcagc ccttggggca gcccatacaa ggccatgggg | 720 |
| ctgggcaagc tgcacgcctg ggtccggggt gggcacggtg cccgggcaac gagctgaaag | 780 |
| ctcatctgct ctcaggggcc cctccctggg gacagcccct cctggctagt cacaccctgt | 840 |
| aggctcctct atataaccca ggggcacagg ggctgccctc attctaccac cacctccaca | 900 |
| gcacagacag acactcagga gccagccagg gtaagtttag tctttttgtc ttttatttca | 960 |
| ggtcccggat ccggtggtgg tgcaaatcaa agaactgctc ctcagtggat gttgccttta | 1020 |
| cttctaggcc tgtacggaag tgttacttct gctctaaaag ctgcggaatt gtacccgcgc | 1080 |
| caccATGagc gccgccaccc actcgcccat gatgcaggtg gcgtccggca acggtgaccg | 1140 |
| cgaccctttg ccccccggat gggagatcaa gatcgacccg cagaccggct ggcccttctt | 1200 |
| cgtggaccac aacagccgca ccactacgtg gaacgacccg cgcgtgccct ctgagggccc | 1260 |
| caaggagact ccatcctctg ccaatggccc ttcccgggag ggctctaggc tgccgcctgc | 1320 |
| tagggaaggc caccctgtgt acccccagct ccgaccaggc tacattccca ttcctgtgct | 1380 |
| ccatgaaggc gctgagaacc ggcaggtgca ccctttccat gtctatcccc agcctgggat | 1440 |
| gcagcgattc cgaactgagg cggcagcagc ggctcctcag aggtcccagt cacctctgcg | 1500 |
| gggcatgcca gaaaccactc agccagataa acagtgtgga caggtggcag cggcggcggc | 1560 |
| agcccagccc ccagcctccc acggacctga gcggtcccag tctccagctg cctctgactg | 1620 |
| ctcatcctca tcctcctcgg ccagcctgcc ttcctccggc aggagcagcc tgggcagtca | 1680 |
| ccagctcccg cgggggtaca tctccattcc ggtgatacac gagcagaacg ttacccggcc | 1740 |
| agcagcccag ccctccttcc accaagccca gaagacgcac tacccagcgc agcaggggga | 1800 |
| gtaccagacc caccagcctg tgtaccacaa gatccagggg gatgactggg agccccggcc | 1860 |
| cctgcgggcg gcatccccgt tcaggtcatc tgtccagggt gcatcgagcc gggagggctc | 1920 |
| accagccagg agcagcacgc cactccactc cccctcgccc atccgtgtgc acaccgtggt | 1980 |
| cgacaggcct cagcagccca tgacccatcg agaaactgca cctgtttccc agcctgaaaa | 2040 |
| caaaccagaa agtaagccag gcccagttgg accagaactc cctcctggac acatcccaat | 2100 |
| tcaagtgatc cgcaaagagg tggattctaa acctgtttcc cagaagcccc cacctccctc | 2160 |
| tgagaaggta gaggtgaaag ttccccctgc tccagttcct tgtcctcctc ccagccctgg | 2220 |
| cccttctgct gtcccctctt cccccaagag tgtggctaca gaagagaggg cagcccccag | 2280 |
| cactgcccct gcagaagcta cacctccaaa accaggagaa gccgaggctc ccccaaaaca | 2340 |
| tccaggagtg ctgaaagtgg aagccatcct ggagaaggta caggggctgg agcaggctgt | 2400 |
| agacaacttt gaaggcaaga agactgacaa aaagtacctg atgatcgaag agtatttgac | 2460 |
| caaagagctg ctggccctgg attcagtgga ccccgaggga cgagccgatg tgcgtcaggc | 2520 |
| caggagagac ggtgtcagga aggttcagac catcttggaa aaacttgaac agaaagccat | 2580 |
| tgatgtccca ggtcaagtcc aggtctatga actccagccc agcaaccttg aagcagatca | 2640 |
| gccactgcag gcaatcatgg agatgggtgc cgtggcagca gacaagggca agaaaaatgc | 2700 |
| tggaaatgca gaagatcccc acacagaaac ccagcagcca gaagccacag cagcagcgac | 2760 |
| ttcaaacccc agcagcatga cagacacccc tggtaaccca gcagcaccgT AGtcaacctc | 2820 |
| tggattacaa aatttgtgaa agattgactg gtattcttaa ctatgttgct ccttttacgc | 2880 |
| tatgtggata cgctgcttta atgcctttgt atcatgctat tgcttcccgt atggctttca | 2940 |
| ttttctcctc cttgtataaa tcctggttgc tgtctcttta tgaggagttg tggcccgttg | 3000 |
| tcaggcaacg tggcgtggtg tgcactgtgt ttgctgacgc aacccccact ggttggggca | 3060 |
| ttgccaccac ctgtcagctc ctttccggga ctttcgcttt ccccctccct attgccacgg | 3120 |
| cggaactcat cgccgcctgc cttgcccgct gctggacagg ggctcggctg ttgggcactg | 3180 |
| acaattccgt ggtgttgtcg gggaaatcat cgtcctttcc ttggctgctc gcctgtgttg | 3240 |
| ccacctggat tctgcgcggg acgtccttct gctacgtccc ttcggccctc aatccagcgg | 3300 |
| accttccttc ccgcggcctg ctgccggctc tgcggcctct tccgcgtctt cgccttcgcc | 3360 |
| ctcagacgag tcggatctcc ctttgggccg cctccccgca ctgcccgggt ggcatccctg | 3420 |
| tgacccctcc ccagtgcctc tcctggccct ggaagttgcc actccagtgc ccaccagcct | 3480 |
| tgtcctaata aaattaagtt gcatcatttt gtctgactag gtgtccttct ataatattat | 3540 |
| ggggtggagg ggggtggtat ggagcaaggg gcccaagttg ggaagaaacc tgtagggcct | 3600 |
| gccctaagga ggaaccccta gtgatggagt tggccactcc ctctctgcgc gctcgctcgc | 3660 |
| tcactgaggc cgggcgacca aaggtcgccc gacgcccggg ctttgcccgg gcggcctcag | 3720 |
| tgagcgagcg agcgcgcaga gagggagtgg ccaa | 3754 |

In a certain embodiment, the vector genome comprises, in 5′ to 3′ order, a 5′ ITR; an hTNNT2 promoter; a BAG3 transgene; an WPRE(x) element; a Human GH poly(A) signal (hGH) sequence; and a 3′ ITR. The vector genome may comprise, in 5′ to 3′ order, the polynucleotide sequences SEQ ID NO: 108; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. In certain embodiments, this vector genome is packaged in an AAVrh74 vector.

| SEQ ID NO: 108 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccAT Gagcgccgcc | 720 |
| acccactcgc ccatgatgca ggtggcgtcc ggcaacggtg accgcgaccc tttgcccccc | 780 |
| ggatgggaga tcaagatcga cccgcagacc ggctggccct tcttcgtgga ccacaacagc | 840 |
| cgcaccacta cgtggaacga cccgcgcgtg ccctctgagg gccccaagga gactccatcc | 900 |
| tctgccaatg gcccttcccg ggagggctct aggctgccgc ctgctaggga aggccaccct | 960 |
| gtgtaccccc agctccgacc aggctacatt cccattcctg tgctccatga aggcgctgag | 1020 |
| aaccggcagg tgcacccttt ccatgtctat ccccagcctg ggatgcagcg attccgaact | 1080 |
| gaggcggcag cagcggctcc tcagaggtcc cagtcacctc tgcggggcat gccagaaacc | 1140 |
| actcagccag ataaacagtg tggacaggtg gcagcggcgg cggcagccca gcccccagcc | 1200 |
| tcccacggac ctgagcggtc ccagtctcca gctgcctctg actgctcatc ctcatcctcc | 1260 |
| tcggccagcc tgccttcctc cggcaggagc agcctgggca gtcaccagct cccgcggggg | 1320 |
| tacatctcca ttccggtgat acacgagcag aacgttaccc ggccagcagc ccagccctcc | 1380 |
| ttccaccaag cccagaagac gcactaccca gcgcagcagg gggagtacca gacccaccag | 1440 |
| cctgtgtacc acaagatcca gggggatgac tgggagcccc ggcccctgcg ggcggcatcc | 1500 |
| ccgttcaggt catctgtcca gggtgcatcg agccgggagg gctcaccagc caggagcagc | 1560 |
| acgccactcc actccccctc gcccatccgt gtgcacaccg tggtcgacag gcctcagcag | 1620 |
| cccatgaccc atcgagaaac tgcacctgtt tcccagcctg aaaacaaacc agaaagtaag | 1680 |
| ccaggcccag ttggaccaga actccctcct ggacacatcc caattcaagt gatccgcaaa | 1740 |
| gaggtggatt ctaaacctgt ttcccagaag cccccacctc cctctgagaa ggtagaggtg | 1800 |
| aaagttcccc ctgctccagt tccttgtcct cctcccagcc ctggcccttc tgctgtcccc | 1860 |
| tcttccccca agagtgtggc tacagaagag agggcagccc ccagcactgc ccctgcagaa | 1920 |
| gctacacctc caaaaccagg agaagccgag gctcccccaa aacatccagg agtgctgaaa | 1980 |
| gtggaagcca tcctggagaa ggtacagggg ctggagcagg ctgtagacaa ctttgaaggc | 2040 |
| aagaagactg acaaaaagta cctgatgatc gaagagtatt tgaccaaaga gctgctggcc | 2100 |
| ctggattcag tggaccccga gggacgagcc gatgtgcgtc aggccaggag agacggtgtc | 2160 |
| aggaaggttc agaccatctt ggaaaaactt gaacagaaag ccattgatgt cccaggtcaa | 2220 |
| gtccaggtct atgaactcca gcccagcaac cttgaagcag atcagccact gcaggcaatc | 2280 |
| atggagatgg gtgccgtggc agcagacaag ggcaagaaaa atgctggaaa tgcagaagat | 2340 |
| ccccacacag aaacccagca gccagaagcc acagcagcag cgacttcaaa ccccagcagc | 2400 |
| atgacagaca cccctggtaa cccagcagca ccgTAGtcaa cctctggatt acaaaatttg | 2460 |
| tgaaagattg actggtattc ttaactatgt tgctcctttt acgctatgtg gatacgctgc | 2520 |
| tttaatgcct ttgtatcatg ctattgcttc ccgtatggct ttcattttct cctccttgta | 2580 |
| taaatcctgg ttgctgtctc tttatgagga gttgtggccc gttgtcaggc aacgtggcgt | 2640 |
| ggtgtgcact gtgtttgctg acgcaacccc cactggttgg ggcattgcca ccacctgtca | 2700 |
| gctcctttcc gggactttcg ctttccccct ccctattgcc acggcggaac tcatcgccgc | 2760 |
| ctgccttgcc cgctgctgga caggggctcg gctgttgggc actgacaatt ccgtggtgtt | 2820 |
| gtcggggaaa tcatcgtcct ttccttggct gctcgcctgt gttgccacct ggattctgcg | 2880 |
| cgggacgtcc ttctgctacg tcccttcggc cctcaatcca gcggaccttc cttcccgcgg | 2940 |
| cctgctgccg gctctgcggc ctcttccgcg tcttcgcctt cgccctcaga cgagtcggat | 3000 |
| ctccctttgg gccgcctccc cgcactgccc gggtggcatc cctgtgaccc ctccccagtg | 3060 |
| cctctcctgg ccctggaagt tgccactcca gtgcccacca gccttgtcct aataaaatta | 3120 |
| agttgcatca ttttgtctga ctaggtgtcc ttctataata ttatggggtg gaggggggtg | 3180 |
| gtatggagca aggggcccaa gttgggaaga aacctgtagg gcctgcccta aggaggaacc | 3240 |
| cctagtgatg gagttggcca ctccctctct gcgcgctcgc tcgctcactg aggccgggcg | 3300 |
| accaaaggtc gcccgacgcc cgggctttgc ccgggcggcc tcagtgagcg agcgagcgcg | 3360 |
| cagagaggga gtggccaa | 3378 |

In a certain embodiment, the vector genome comprises, in 5′ to 3′ order, a 5′ ITR; an HSP70 promoter; a BAG3 transgene; an WPRE(x) element; a Human GH poly(A) signal (hGH) sequence; and a 3′ ITR. The vector genome may comprise, in 5′ to 3′ order, the polynucleotide sequences SEQ ID NO: 109; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. In certain embodiments, this vector genome is packaged in an AAVrh74 vector.

| SEQ ID NO: 109 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtcc tgcagggccg cccactcccc | 180 |
| cttcctctca gggtccctgt cccctccagt gaatcccaga agactctgga gagttctgag | 240 |
| cagggggcgg cactctggcc tctgattggt ccaaggaagg ctggggggca ggacgggagg | 300 |
| cgaaaaccct ggaatattcc cgacctggca gcctcatcga gctcggtgat tggctcagaa | 360 |
| gggaaaaggc gggtctccgt gacgacttat aaaagcccag gggcaagcgg tccggataac | 420 |
| ggctagcctg aggagctgct gcgacagtcc actacctttt tcgagagtga ctcccgttgt | 480 |
| cccaaggctt cccagagcga acctgtgcgg ctgcaggcac cggcgcgtcg agtttccggc | 540 |
| gtccggaagg accgagctct tctcgcggat ccagtgttcc gtttccagcc cccaatctca | 600 |
| gagcggagcc gacagagagc agggaacccg ccaccATGag cgccgccacc cactcgccca | 660 |
| tgatgcaggt ggcgtccggc aacggtgacc gcgacccttt gccccccgga tgggagatca | 720 |
| agatcgaccc gcagaccggc tggcccttct tcgtggacca caacagccgc accactacgt | 780 |
| ggaacgaccc gcgcgtgccc tctgagggcc ccaaggagac tccatcctct gccaatggcc | 840 |
| cttcccggga gggctctagg ctgccgcctg ctagggaagg ccaccctgtg tacccccagc | 900 |
| tccgaccagg ctacattccc attcctgtgc tccatgaagg cgctgagaac cggcaggtgc | 960 |
| accctttcca tgtctatccc cagcctggga tgcagcgatt ccgaactgag gcggcagcag | 1020 |
| cggctcctca gaggtcccag tcacctctgc ggggcatgcc agaaaccact cagccagata | 1080 |
| aacagtgtgg acaggtggca gcggcggcgg cagcccagcc cccagcctcc cacggacctg | 1140 |
| agcggtccca gtctccagct gcctctgact gctcatcctc atcctcctcg gccagcctgc | 1200 |
| cttcctccgg caggagcagc ctgggcagtc accagctccc gcgggggtac atctccattc | 1260 |
| cggtgataca cgagcagaac gttacccggc cagcagccca gccctccttc caccaagccc | 1320 |
| agaagacgca ctacccagcg cagcaggggg agtaccagac ccaccagcct gtgtaccaca | 1380 |
| agatccaggg ggatgactgg gagccccggc ccctgcgggc ggcatccccg ttcaggtcat | 1440 |
| ctgtccaggg tgcatcgagc cgggagggct caccagccag gagcagcacg ccactccact | 1500 |
| ccccctcgcc catccgtgtg cacaccgtgg tcgacaggcc tcagcagccc atgacccatc | 1560 |
| gagaaactgc acctgtttcc cagcctgaaa acaaaccaga aagtaagcca ggcccagttg | 1620 |
| gaccagaact ccctcctgga cacatcccaa ttcaagtgat ccgcaaagag gtggattcta | 1680 |
| aacctgtttc ccagaagccc ccacctccct ctgagaaggt agaggtgaaa gttccccctg | 1740 |
| ctccagttcc ttgtcctcct cccagccctg gcccttctgc tgtcccctct tcccccaaga | 1800 |
| gtgtggctac agaagagagg gcagccccca gcactgcccc tgcagaagct acacctccaa | 1860 |
| aaccaggaga agccgaggct cccccaaaac atccaggagt gctgaaagtg gaagccatcc | 1920 |
| tggagaaggt acaggggctg gagcaggctg tagacaactt tgaaggcaag aagactgaca | 1980 |
| aaaagtacct gatgatcgaa gagtatttga ccaaagagct gctggccctg gattcagtgg | 2040 |
| accccgaggg acgagccgat gtgcgtcagg ccaggagaga cggtgtcagg aaggttcaga | 2100 |
| ccatcttgga aaaacttgaa cagaaagcca ttgatgtccc aggtcaagtc caggtctatg | 2160 |
| aactccagcc cagcaacctt gaagcagatc agccactgca ggcaatcatg gagatgggtg | 2220 |
| ccgtggcagc agacaagggc aagaaaaatg ctggaaatgc agaagatccc cacacagaaa | 2280 |
| cccagcagcc agaagccaca gcagcagcga cttcaaaccc cagcagcatg acagacaccc | 2340 |
| ctggtaaccc agcagcaccg TAGtcaacct ctggattaca aaatttgtga aagattgact | 2400 |
| ggtattctta actatgttgc tccttttacg ctatgtggat acgctgcttt aatgcctttg | 2460 |
| tatcatgcta ttgcttcccg tatggctttc attttctcct ccttgtataa atcctggttg | 2520 |
| ctgtctcttt atgaggagtt gtggcccgtt gtcaggcaac gtggcgtggt gtgcactgtg | 2580 |
| tttgctgacg caacccccac tggttggggc attgccacca cctgtcagct cctttccggg | 2640 |
| actttcgctt tccccctccc tattgccacg gcggaactca tcgccgcctg ccttgcccgc | 2700 |
| tgctggacag gggctcggct gttgggcact gacaattccg tggtgttgtc ggggaaatca | 2760 |
| tcgtcctttc cttggctgct cgcctgtgtt gccacctgga ttctgcgcgg gacgtccttc | 2820 |
| tgctacgtcc cttcggccct caatccagcg gaccttcctt cccgcggcct gctgccggct | 2880 |
| ctgcggcctc ttccgcgtct tcgccttcgc cctcagacga gtcggatctc cctttgggcc | 2940 |
| gcctccccgc actgcccggg tggcatccct gtgacccctc cccagtgcct ctcctggccc | 3000 |
| tggaagttgc cactccagtg cccaccagcc ttgtcctaat aaaattaagt tgcatcattt | 3060 |
| tgtctgacta ggtgtccttc tataatatta tggggtggag gggggtggta tggagcaagg | 3120 |
| ggcccaagtt gggaagaaac ctgtagggcc tgccctaagg aggaacccct agtgatggag | 3180 |
| ttggccactc cctctctgcg cgctcgctcg ctcactgagg ccgggcgacc aaaggtcgcc | 3240 |
| cgacgcccgg gctttgcccg ggcggcctca gtgagcgagc gagcgcgcag agagggagtg | 3300 |
| gccaa | 3305 |

In a certain embodiment, the vector genome comprises, in 5′ to 3′ order, a 5′ ITR; an MHCK7 promoter; an SV-40 Chimeric Intron; a BAG3 transgene; enhanced green fluorescent protein (eGFP); an WPRE(x) element; a Human GH poly(A) signal (hGH) sequence; and a 3′ ITR. The vector genome may comprise, in 5′ to 3′ order, the polynucleotide sequences SEQ ID NO: 110; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. In certain embodiments, this vector genome is packaged in an AAVrh74 vector.

SEQ ID NO: 110
ttggccactccctctctgcgcgctcgctcgctcactgaggccgcccgggcaaagcccgggcgtcgggcgacctttggtcgccc
ggcctcagtgagcgagcgagcgcgcagagagggagtggccaactccatcactaggggttcctcctgaggacgcgtacccttca
gattaaaaataactgaggtaagggcctgggtaggggaggtggtgtgagacgctcctgtctctcctctatctgcccatcggccc
tttggggaggaggaatgtgcccaaggactaaaaaaaggccatggagccagaggggcgagggcaacagacctttcatgggcaaa
ccttggggccctgctgtctagcatgccccactacgggtctaggctgcccatgtaaggaggcaaggcctggggacacccgagat
gcctggttataattaacccagacatgtggctgcccccccccccccaacacctgctgcctctaaaaataaccctgtccctggtg
gatcccctgcatgcgaagatcttcgaacaaggctgtgggggactgagggcaggctgtaacaggcttgggggccagggcttata
cgtgcctgggactcccaaagtattactgttccatgttcccggcgaagggccagctgtcccccgccagctagactcagcactta
gtttaggaaccagtgagcaagtcagcccttggggcagcccatacaaggccatggggctgggcaagctgcacgcctgggtccgg
ggtgggcacggtgcccgggcaacgagctgaaagctcatctgctctcaggggcccctccctggggacagcccctcctggctagt
cacaccctgtaggctcctctatataacccaggggcacaggggctgccctcattctaccaccacctccacagcacagacagaca
ctcaggagccagccagggtaagtttagtctttttgtcttttatttcaggtcccggatccggtggtggtgcaaatcaaagaact
gctcctcagtggatgttgcctttacttctaggcctgtacggaagtgttacttctgctctaaaagctgcggaattgtacccgcg
ccaccATGagcgccgccacccactcgcccatgatgcaggtggcgtccggcaacggtgaccgcgaccctttgccccccggatgg
gagatcaagatcgacccgcagaccggctggcccttcttcgtggaccacaacagccgcaccactacgtggaacgacccgcgcgt
gccctctgagggccccaaggagactccatcctctgccaatggcccttcccgggagggctctaggctgccgcctgctagggaag
gccaccctgtgtacccccagctccgaccaggctacattcccattcctgtgctccatgaaggcgctgagaaccggcaggtgcac
cctttccatgtctatccccagcctgggatgcagcgattccgaactgaggcggcagcagcggctcctcagaggtcccagtcacc
tctgcggggcatgccagaaaccactcagccagataaacagtgtggacaggtggcagcggcggcggcagcccagcccccagcct
cccacggacctgagcggtcccagtctccagctgcctctgactgctcatcctcatcctcctcggccagcctgccttcctccggc
aggagcagcctgggcagtcaccagctcccgcgggggtacatctccattccggtgatacacgagcagaacgttacccggccagc
agcccagccctccttccaccaagcccagaagacgcactacccagcgcagcagggggagtaccagacccaccagcctgtgtacc
acaagatccagggggatgactgggagccccggcccctgcgggcggcatccccgttcaggtcatctgtccagggtgcatcgagc
cgggagggctcaccagccaggagcagcacgccactccactccccctcgcccatccgtgtgcacaccgtggtcgacaggcctca
gcagcccatgacccatcgagaaactgcacctgtttcccagcctgaaaacaaaccagaaagtaagccaggcccagttggaccag
aactccctcctggacacatcccaattcaagtgatccgcaaagaggtggattctaaacctgtttcccagaagcccccacctccc
tctgagaaggtagaggtgaaagttccccctgctccagttccttgtcctcctcccagccctggcccttctgctgtcccctcttc
ccccaagagtgtggctacagaagagagggcagcccccagcactgcccctgcagaagctacacctccaaaaccaggagaagccg
aggctcccccaaaacatccaggagtgctgaaagtggaagccatcctggagaaggtacaggggctggagcaggctgtagacaac
tttgaaggcaagaagactgacaaaaagtacctgatgatcgaagagtatttgaccaaagagctgctggccctggattcagtgga
ccccgagggacgagccgatgtgcgtcaggccaggagagacggtgtcaggaaggttcagaccatcttggaaaaacttgaacaga
aagccattgatgtcccaggtcaagtccaggtctatgaactccagcccagcaaccttgaagcagatcagccactgcaggcaatc
atggagatgggtgccgtggcagcagacaagggcaagaaaaatgctggaaatgcagaagatccccacacagaaacccagcagcc
agaagccacagcagcagcgacttcaaaccccagcagcatgacagacacccctggtaacccagcagcaccgggatccggcagtg
gagagggcagaggaagtctgctaacatgcggtgacgtcgaggagaatcctggcccaATGAGCAAGGGCGAGGAGCTGTTCACC
GGCGTGGTGCCCATCCTGGTGGAGCTGGACGGCGACGTGAACGGCCACAAGTTCAGCGTGAGAGGCGAGGGCGAGGGCGACGC
CACCAACGGCAAGCTGACCCTGAAGTTCATCTGCACCACCGGCAAGCTGCCCGTGCCCTGGCCCACCCTGGTGACCACCCTGA
CCTACGGCGTGCTGTGCTTCAGCAGATACCCCGACCACATGAAGAGACACGACTTCTTCAAGAGCGCCATGCCCGAGGGCTAC
GTGCAGGAGAGAACCATCAGCTTCAAGGACGACGGCACCTACAAGACCAGAGCCGAGGTGAAGTTCGAGGGCGACACCCTGGT
GAACAGAATCGAGCTGAAGGGCATCGACTTCAAGGAGGACGGCAACATCCTGGGCCACAAGCTGGAGTACAACTTCAACAGCC
ACAACGTGTACATCACCGCCGACAAGCAGAAGAACGGCATCAAGGCCTACTTCAAGATCAGACACAACGTGGAGGACGGCAGC
GTGCAGCTGGCCGACCACTACCAGCAGAACACCCCCATCGGCGACGGCCCCGTGCTGCTGCCCGACAACCACTACCTGAGCAC
CCAGAGCGTGCTGAGCAAGGACCCCAACGAGAAGAGAGACCACATGGTGCTGCTGGAGGACGTGACCGCCGCCGGCATCACCC
ACGGCATGGACGAGCTGTACAAGTGAtcaacctctggattacaaaatttgtgaaagattgactggtattcttaactatgttgc
tccttttacgctatgtggatacgctgctttaatgcctttgtatcatgctattgcttcccgtatggctttcattttctcctcct
tgtataaatcctggttgctgtctctttatgaggagttgtggcccgttgtcaggcaacgtggcgtggtgtgcactgtgtttgct
gacgcaacccccactggttggggcattgccaccacctgtcagctcctttccgggactttcgctttccccctccctattgccac
ggcggaactcatcgccgcctgccttgcccgctgctggacaggggctcggctgttgggcactgacaattccgtggtgttgtcgg
ggaaatcatcgtcctttccttggctgctcgcctgtgttgccacctggattctgcgcgggacgtccttctgctacgtcccttcg
gccctcaatccagcggaccttccttcccgcggcctgctgccggctctgcggcctcttccgcgtcttcgccttcgccctcagac
gagtcggatctccctttgggccgcctccccgcactgcccgggtggcatccctgtgacccctccccagtgcctctcctggccct
ggaagttgccactccagtgcccaccagccttgtcctaataaaattaagttgcatcattttgtctgactaggtgtccttctata
atattatggggtggaggggggtggtatggagcaaggggcccaagttgggaagaaacctgtagggcctgccctaaggaggaacc
cctagtgatggagttggccactccctctctgcgcgctcgctcgctcactgaggccgggcgaccaaaggtcgcccgacgcccgg
gctttgcccgggcggcctcagtgagcgagcgagcgcgcagagagggagtggccaa

In a certain embodiment, the vector genome comprises, in 5′ to 3′ order, a 5′ ITR; an TNNT2 promoter; a BAG3 transgene; enhanced green fluorescent protein (eGFP); an WPRE(x) element; a Human GH poly(A) signal (hGH) sequence; and a 3′ ITR. The vector genome may comprise, in 5′ to 3′ order, the polynucleotide sequences SEQ ID NO: 111; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. In certain embodiments, this vector genome is packaged in an AAVrh74 vector.

| SEQ ID NO: 111 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtct cagtccatta ggagccagta | 180 |
| gcctggaaga tgtctttacc cccagcatca gttcaagtgg agcagcacat aactcttgcc | 240 |
| ctctgccttc caagattctg gtgctgagac ttatggagtg tcttggaggt tgccttctgc | 300 |
| cccccaaccc tgctcccagc tggccctccc aggcctgggt tgctggcctc tgctttatca | 360 |
| ggattctcaa gagggacagc tggtttatgt tgcatgactg ttccctgcat atctgctctg | 420 |
| gttttaaata gcttatctga gcagctggag gaccacatgg gcttatatgg cgtggggtac | 480 |
| atgttcctgt agccttgtcc ctggcacctg ccaaaatagc agccaacacc ccccaccccc | 540 |
| accgccatcc ccctgcccca cccgtcccct gtcgcacatt cctccctccg cagggctggc | 600 |
| tcaccaggcc ccagcccaca tgcctgctta aagccctctc catcctctgc ctcacccagt | 660 |
| ccccgctgag actgagcaga cgcctccagg atctgtcggc aggccaccAT Gagcgccgcc | 720 |
| acccactcgc ccatgatgca ggtggcgtcc ggcaacggtg accgcgaccc tttgcccccc | 780 |
| ggatgggaga tcaagatcga cccgcagacc ggctggccct tcttcgtgga ccacaacagc | 840 |
| cgcaccacta cgtggaacga cccgcgcgtg ccctctgagg gccccaagga gactccatcc | 900 |
| tctgccaatg gcccttcccg ggagggctct aggctgccgc ctgctaggga aggccaccct | 960 |
| gtgtaccccc agctccgacc aggctacatt cccattcctg tgctccatga aggcgctgag | 1020 |
| aaccggcagg tgcacccttt ccatgtctat ccccagcctg ggatgcagcg attccgaact | 1080 |
| gaggcggcag cagcggctcc tcagaggtcc cagtcacctc tgcggggcat gccagaaacc | 1140 |
| actcagccag ataaacagtg tggacaggtg gcagcggcgg cggcagccca gcccccagcc | 1200 |
| tcccacggac ctgagcggtc ccagtctcca gctgcctctg actgctcatc ctcatcctcc | 1260 |
| tcggccagcc tgccttcctc cggcaggagc agcctgggca gtcaccagct cccgcggggg | 1320 |
| tacatctcca ttccggtgat acacgagcag aacgttaccc ggccagcagc ccagccctcc | 1380 |
| ttccaccaag cccagaagac gcactaccca gcgcagcagg gggagtacca gacccaccag | 1440 |
| cctgtgtacc acaagatcca gggggatgac tgggagcccc ggcccctgcg ggcggcatcc | 1500 |
| ccgttcaggt catctgtcca gggtgcatcg agccgggagg gctcaccagc caggagcagc | 1560 |
| acgccactcc actccccctc gcccatccgt gtgcacaccg tggtcgacag gcctcagcag | 1620 |
| cccatgaccc atcgagaaac tgcacctgtt tcccagcctg aaaacaaacc agaaagtaag | 1680 |
| ccaggcccag ttggaccaga actccctcct ggacacatcc caattcaagt gatccgcaaa | 1740 |
| gaggtggatt ctaaacctgt ttcccagaag cccccacctc cctctgagaa ggtagaggtg | 1800 |
| aaagttcccc ctgctccagt tccttgtcct cctcccagcc ctggcccttc tgctgtcccc | 1860 |
| tcttccccca agagtgtggc tacagaagag agggcagccc ccagcactgc ccctgcagaa | 1920 |
| gctacacctc caaaaccagg agaagccgag gctcccccaa aacatccagg agtgctgaaa | 1980 |
| gtggaagcca tcctggagaa ggtacagggg ctggagcagg ctgtagacaa ctttgaaggc | 2040 |
| aagaagactg acaaaaagta cctgatgatc gaagagtatt tgaccaaaga gctgctggcc | 2100 |
| ctggattcag tggaccccga gggacgagcc gatgtgcgtc aggccaggag agacggtgtc | 2160 |
| aggaaggttc agaccatctt ggaaaaactt gaacagaaag ccattgatgt cccaggtcaa | 2220 |
| gtccaggtct atgaactcca gcccagcaac cttgaagcag atcagccact gcaggcaatc | 2280 |
| atggagatgg gtgccgtggc agcagacaag ggcaagaaaa atgctggaaa tgcagaagat | 2340 |
| ccccacacag aaacccagca gccagaagcc acagcagcag cgacttcaaa ccccagcagc | 2400 |
| atgacagaca cccctggtaa cccagcagca ccgggatccg gcagtggaga gggcagagga | 2460 |
| agtctgctaa catgcggtga cgtcgaggag aatcctggcc caATGAGCAA GGGCGAGGAG | 2520 |
| CTGTTCACCG GCGTGGTGCC CATCCTGGTG GAGCTGGACG GCGACGTGAA CGGCCACAAG | 2580 |
| TTCAGCGTGA GAGGCGAGGG CGAGGGCGAC GCCACCAACG GCAAGCTGAC CCTGAAGTTC | 2640 |
| ATCTGCACCA CCGGCAAGCT GCCCGTGCCC TGGCCCACCC TGGTGACCAC CCTGACCTAC | 2700 |
| GGCGTGCTGT GCTTCAGCAG ATACCCCGAC CACATGAAGA GACACGACTT CTTCAAGAGC | 2760 |
| GCCATGCCCG AGGGCTACGT GCAGGAGAGA ACCATCAGCT TCAAGGACGA CGGCACCTAC | 2820 |
| AAGACCAGAG CCGAGGTGAA GTTCGAGGGC GACACCCTGG TGAACAGAAT CGAGCTGAAG | 2880 |
| GGCATCGACT TCAAGGAGGA CGGCAACATC CTGGGCCACA AGCTGGAGTA CAACTTCAAC | 2940 |
| AGCCACAACG TGTACATCAC CGCCGACAAG CAGAAGAACG GCATCAAGGC CTACTTCAAG | 3000 |
| ATCAGACACA ACGTGGAGGA CGGCAGCGTG CAGCTGGCCG ACCACTACCA GCAGAACACC | 3060 |
| CCCATCGGCG ACGGCCCCGT GCTGCTGCCC GACAACCACT ACCTGAGCAC CCAGAGCGTG | 3120 |
| CTGAGCAAGG ACCCCAACGA GAAGAGAGAC CACATGGTGC TGCTGGAGGA CGTGACCGCC | 3180 |
| GCCGGCATCA CCCACGGCAT GGACGAGCTG TACAAGTGAt caacctctgg attacaaaat | 3240 |
| ttgtgaaaga ttgactggta ttcttaacta tgttgctcct tttacgctat gtggatacgc | 3300 |
| tgctttaatg cctttgtatc atgctattgc ttcccgtatg gctttcattt tctcctcctt | 3360 |
| gtataaatcc tggttgctgt ctctttatga ggagttgtgg cccgttgtca ggcaacgtgg | 3420 |
| cgtggtgtgc actgtgtttg ctgacgcaac ccccactggt tggggcattg ccaccacctg | 3480 |
| tcagctcctt tccgggactt tcgctttccc cctccctatt gccacggcgg aactcatcgc | 3540 |
| cgcctgcctt gcccgctgct ggacaggggc tcggctgttg ggcactgaca attccgtggt | 3600 |
| gttgtcgggg aaatcatcgt cctttccttg gctgctcgcc tgtgttgcca cctggattct | 3660 |
| gcgcgggacg tccttctgct acgtcccttc ggccctcaat ccagcggacc ttccttcccg | 3720 |
| cggcctgctg ccggctctgc ggcctcttcc gcgtcttcgc cttcgccctc agacgagtcg | 3780 |
| gatctccctt tgggccgcct ccccgcactg cccgggtggc atccctgtga cccctcccca | 3840 |
| gtgcctctcc tggccctgga agttgccact ccagtgccca ccagccttgt cctaataaaa | 3900 |
| ttaagttgca tcattttgtc tgactaggtg tccttctata atattatggg gtggaggggg | 3960 |
| gtggtatgga gcaaggggcc caagttggga agaaacctgt agggcctgcc ctaaggagga | 4020 |
| acccctagtg atggagttgg ccactccctc tctgcgcgct cgctcgctca ctgaggccgg | 4080 |
| gcgaccaaag gtcgcccgac gcccgggctt tgcccgggcg gcctcagtga gcgagcgagc | 4140 |
| gcgcagagag ggagtggcca a | 4161 |

In a certain embodiment, the vector genome comprises, in 5′ to 3′ order, a 5′ ITR; an HSP70 promoter; a BAG3 transgene; enhanced green fluorescent protein (eGFP); an WPRE(x) element; a Human GH poly(A) signal (hGH) sequence; and a 3′ ITR. The vector genome may comprise, in 5′ to 3′ order, the polynucleotide sequences SEQ ID NO: 112; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. In certain embodiments, this vector genome is packaged in an AAVrh74 vector.

| SEQ ID NO: 112 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtcc tgcagggccg cccactcccc | 180 |
| cttcctctca gggtccctgt cccctccagt gaatcccaga agactctgga gagttctgag | 240 |
| cagggggcgg cactctggcc tctgattggt ccaaggaagg ctggggggca ggacgggagg | 300 |
| cgaaaaccct ggaatattcc cgacctggca gcctcatcga gctcggtgat tggctcagaa | 360 |
| gggaaaaggc gggtctccgt gacgacttat aaaagcccag gggcaagcgg tccggataac | 420 |
| ggctagcctg aggagctgct gcgacagtcc actacctttt tcgagagtga ctcccgttgt | 480 |
| cccaaggctt cccagagcga acctgtgcgg ctgcaggcac cggcgcgtcg agtttccggc | 540 |
| gtccggaagg accgagctct tctcgcggat ccagtgttcc gtttccagcc cccaatctca | 600 |
| gagcggagcc gacagagagc agggaacccg ccaccATGag cgccgccacc cactcgccca | 660 |
| tgatgcaggt ggcgtccggc aacggtgacc gcgacccttt gccccccgga tgggagatca | 720 |
| agatcgaccc gcagaccggc tggcccttct tcgtggacca caacagccgc accactacgt | 780 |
| ggaacgaccc gcgcgtgccc tctgagggcc ccaaggagac tccatcctct gccaatggcc | 840 |
| cttcccggga gggctctagg ctgccgcctg ctagggaagg ccaccctgtg tacccccagc | 900 |
| tccgaccagg ctacattccc attcctgtgc tccatgaagg cgctgagaac cggcaggtgc | 960 |
| accctttcca tgtctatccc cagcctggga tgcagcgatt ccgaactgag gcggcagcag | 1020 |
| cggctcctca gaggtcccag tcacctctgc ggggcatgcc agaaaccact cagccagata | 1080 |
| aacagtgtgg acaggtggca gcggcggcgg cagcccagcc cccagcctcc cacggacctg | 1140 |
| agcggtccca gtctccagct gcctctgact gctcatcctc atcctcctcg gccagcctgc | 1200 |
| cttcctccgg caggagcagc ctgggcagtc accagctccc gcgggggtac atctccattc | 1260 |
| cggtgataca cgagcagaac gttacccggc cagcagccca gccctccttc caccaagccc | 1320 |
| agaagacgca ctacccagcg cagcaggggg agtaccagac ccaccagcct gtgtaccaca | 1380 |
| agatccaggg ggatgactgg gagccccggc ccctgcgggc ggcatccccg ttcaggtcat | 1440 |
| ctgtccaggg tgcatcgagc cgggagggct caccagccag gagcagcacg ccactccact | 1500 |
| ccccctcgcc catccgtgtg cacaccgtgg tcgacaggcc tcagcagccc atgacccatc | 1560 |
| gagaaactgc acctgtttcc cagcctgaaa acaaaccaga aagtaagcca ggcccagttg | 1620 |
| gaccagaact ccctcctgga cacatcccaa ttcaagtgat ccgcaaagag gtggattcta | 1680 |
| aacctgtttc ccagaagccc ccacctccct ctgagaaggt agaggtgaaa gttccccctg | 1740 |
| ctccagttcc ttgtcctcct cccagccctg gcccttctgc tgtcccctct tcccccaaga | 1800 |
| gtgtggctac agaagagagg gcagccccca gcactgcccc tgcagaagct acacctccaa | 1860 |
| aaccaggaga agccgaggct cccccaaaac atccaggagt gctgaaagtg gaagccatcc | 1920 |
| tggagaaggt acaggggctg gagcaggctg tagacaactt tgaaggcaag aagactgaca | 1980 |
| aaaagtacct gatgatcgaa gagtatttga ccaaagagct gctggccctg gattcagtgg | 2040 |
| accccgaggg acgagccgat gtgcgtcagg ccaggagaga cggtgtcagg aaggttcaga | 2100 |
| ccatcttgga aaaacttgaa cagaaagcca ttgatgtccc aggtcaagtc caggtctatg | 2160 |
| aactccagcc cagcaacctt gaagcagatc agccactgca ggcaatcatg gagatgggtg | 2220 |
| ccgtggcagc agacaagggc aagaaaaatg ctggaaatgc agaagatccc cacacagaaa | 2280 |
| cccagcagcc agaagccaca gcagcagcga cttcaaaccc cagcagcatg acagacaccc | 2340 |
| ctggtaaccc agcagcaccg ggatccggca gtggagaggg cagaggaagt ctgctaacat | 2400 |
| gcggtgacgt cgaggagaat cctggcccaA TGAGCAAGGG CGAGGAGCTG TTCACCGGCG | 2460 |
| TGGTGCCCAT CCTGGTGGAG CTGGACGGCG ACGTGAACGG CCACAAGTTC AGCGTGAGAG | 2520 |
| GCGAGGGCGA GGGCGACGCC ACCAACGGCA AGCTGACCCT GAAGTTCATC TGCACCACCG | 2580 |
| GCAAGCTGCC CGTGCCCTGG CCCACCCTGG TGACCACCCT GACCTACGGC GTGCTGTGCT | 2640 |
| TCAGCAGATA CCCCGACCAC ATGAAGAGAC ACGACTTCTT CAAGAGCGCC ATGCCCGAGG | 2700 |
| GCTACGTGCA GGAGAGAACC ATCAGCTTCA AGGACGACGG CACCTACAAG ACCAGAGCCG | 2760 |
| AGGTGAAGTT CGAGGGCGAC ACCCTGGTGA ACAGAATCGA GCTGAAGGGC ATCGACTTCA | 2820 |
| AGGAGGACGG CAACATCCTG GGCCACAAGC TGGAGTACAA CTTCAACAGC CACAACGTGT | 2880 |
| ACATCACCGC CGACAAGCAG AAGAACGGCA TCAAGGCCTA CTTCAAGATC AGACACAACG | 2940 |
| TGGAGGACGG CAGCGTGCAG CTGGCCGACC ACTACCAGCA GAACACCCCC ATCGGCGACG | 3000 |
| GCCCCGTGCT GCTGCCCGAC AACCACTACC TGAGCACCCA GAGCGTGCTG AGCAAGGACC | 3060 |
| CCAACGAGAA GAGAGACCAC ATGGTGCTGC TGGAGGACGT GACCGCCGCC GGCATCACCC | 3120 |
| ACGGCATGGA CGAGCTGTAC AAGTGAtcaa cctctggatt acaaaatttg tgaaagattg | 3180 |
| actggtattc ttaactatgt tgctcctttt acgctatgtg gatacgctgc tttaatgcct | 3240 |
| ttgtatcatg ctattgcttc ccgtatggct ttcattttct cctccttgta taaatcctgg | 3300 |
| ttgctgtctc tttatgagga gttgtggccc gttgtcaggc aacgtggcgt ggtgtgcact | 3360 |
| gtgtttgctg acgcaacccc cactggttgg ggcattgcca ccacctgtca gctcctttcc | 3420 |
| gggactttcg ctttccccct ccctattgcc acggcggaac tcatcgccgc ctgccttgcc | 3480 |
| cgctgctgga caggggctcg gctgttgggc actgacaatt ccgtggtgtt gtcggggaaa | 3540 |
| tcatcgtcct ttccttggct gctcgcctgt gttgccacct ggattctgcg cgggacgtcc | 3600 |
| ttctgctacg tcccttcggc cctcaatcca gcggaccttc cttcccgcgg cctgctgccg | 3660 |
| gctctgcggc ctcttccgcg tcttcgcctt cgccctcaga cgagtcggat ctccctttgg | 3720 |
| gccgcctccc cgcactgccc gggtggcatc cctgtgaccc ctccccagtg cctctcctgg | 3780 |
| ccctggaagt tgccactcca gtgcccacca gccttgtcct aataaaatta agttgcatca | 3840 |
| ttttgtctga ctaggtgtcc ttctataata ttatggggtg gaggggggtg gtatggagca | 3900 |
| aggggcccaa gttgggaaga aacctgtagg gcctgcccta aggaggaacc cctagtgatg | 3960 |
| gagttggcca ctccctctct gcgcgctcgc tcgctcactg aggccgggcg accaaaggtc | 4020 |
| gcccgacgcc cgggctttgc ccgggcggcc tcagtgagcg agcgagcgcg cagagaggga | 4080 |
| gtggccaa | 4088 |

In a certain embodiment, the vector genome comprises, in 5′ to 3′ order, a 5′ ITR; a CMV enhancer, a chicken β-actin promoter; a chimeric intron; a BAG3 transgene; enhanced green fluorescent protein (eGFP); an WPRE(x) element; a Human GH poly(A) signal (hGH) sequence; and a 3′ ITR. The vector genome may comprise, in 5′ to 3′ order, the polynucleotide sequences SEQ ID NO: 113; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. In certain embodiments, this vector genome is packaged in an AAVrh74 vector.

| SEQ ID NO: 113 | |
|---|---|
| ttggccactc cctctctgcg cgctcgctcg ctcactgagg ccgcccgggc aaagcccggg | 60 |
| cgtcgggcga cctttggtcg cccggcctca gtgagcgagc gagcgcgcag agagggagtg | 120 |
| gccaactcca tcactagggg ttcctcctga ggacgcgtta cataacttac ggtaaatggc | 180 |
| ccgcctggct gaccgcccaa cgacccccgc ccattgacgt caataatgac gtatgttccc | 240 |
| atagtaacgc caatagggac tttccattga cgtcaatggg tggagtattt acggtaaact | 300 |
| gcccacttgg cagtacatca agtgtatcat atgccaagta cgccccctat tgacgtcaat | 360 |
| gacggtaaat ggcccgcctg gcattatgcc cagtacatga ccttatggga ctttcctact | 420 |
| tggcagtaca tctacgtatt agtcatcgct attaccatgg tcgaggtgag ccccacgttc | 480 |
| tgcttcactc tccccatctc ccccccctcc ccacccccaa ttttgtattt atttattttt | 540 |
| taattatttt gtgcagcgat gggggcgggg gggggggggg cgcgcgccag gcggggcggg | 600 |
| gcggggcgag gggcggggcg gggcgaggcg gagaggtgcg gcggcagcca atcagagcgg | 660 |
| cgcgctccga aagtttcctt ttatggcgag gcggcggcgg cggcggccct ataaaaagcg | 720 |
| aagcgcgcgg cgggcgggag tcgctgcgcg ctgccttcgc cccgtgcccc gctccgccgc | 780 |
| cgcctcgcgc cgcccgcccc ggctctgact gaccgcgtta ctcccacagg tgagcgggcg | 840 |
| ggacggccct tctcctccgg gctgtaatta gcgcttggtt taatgacggc ttgtttcttt | 900 |
| tctgtggctg cgtgaaagcc ttgaggggct ccgggagggc cctttgtgcg gggggagcgg | 960 |
| ctcggggggt gcgtgcgtgt gtgtgtgcgt ggggagcgcc gcgtgcggct ccgcgctgcc | 1020 |
| cggcggctgt gagcgctgcg ggcgcggcgc ggggctttgt gcgctccgca gtgtgcgcga | 1080 |
| ggggagcgcg gccgggggcg gtgccccgcg gtgcgggggg ggctgcgagg ggaacaaagg | 1140 |
| ctgcgtgcgg ggtgtgtgcg tgggggggtg agcagggggt gtgggcgcgt cggtcgggct | 1200 |
| gcaacccccc ctgcaccccc ctccccgagt tgctgagcac ggcccggctt cgggtgcggg | 1260 |
| gctccgtacg gggcgtggcg cggggctcgc cgtgccgggc ggggggtggc ggcaggtggg | 1320 |
| ggtgccgggc ggggcggggc cgcctcgggc cggggagggc tcgggggagg ggcgcggcgg | 1380 |
| cccccggagc gccggcggct gtcgaggcgc ggcgagccgc agccattgcc ttttatggta | 1440 |
| atcgtgcgag agggcgcagg gacttccttt gtcccaaatc tgtgcggagc cgaaatctgg | 1500 |
| gaggcgccgc cgcaccccct ctagcgggcg cggggcgaag cggtgcggcg ccggcaggaa | 1560 |
| ggaaatgggc ggggagggcc ttcgtgcgtc gccgcgccgc cgtccccttc tccctctcca | 1620 |
| gcctcggggc tgtccgcggg gggacggctg ccttcggggg ggacggggca gggcggggtt | 1680 |
| cggcttctgg cgtgtgaccg gcggctctag agcctctgct aaccatgttc atgccttctt | 1740 |
| ctttttccta cagctcctgg gcaacgtgct ggttattgtg ctgtctcatc attttggcaa | 1800 |
| agaattcATG agcgccgcca cccactcgcc catgatgcag gtggcgtccg gcaacggtga | 1860 |
| ccgcgaccct ttgccccccg gatgggagat caagatcgac ccgcagaccg gctggccctt | 1920 |
| cttcgtggac cacaacagcc gcaccactac gtggaacgac ccgcgcgtgc cctctgaggg | 1980 |
| ccccaaggag actccatcct ctgccaatgg cccttcccgg gagggctcta ggctgccgcc | 2040 |
| tgctagggaa ggccaccctg tgtaccccca gctccgacca ggctacattc ccattcctgt | 2100 |
| gctccatgaa ggcgctgaga accggcaggt gcaccctttc catgtctatc cccagcctgg | 2160 |
| gatgcagcga ttccgaactg aggcggcagc agcggctcct cagaggtccc agtcacctct | 2220 |
| gcggggcatg ccagaaacca ctcagccaga taaacagtgt ggacaggtgg cagcggcggc | 2280 |
| ggcagcccag cccccagcct cccacggacc tgagcggtcc cagtctccag ctgcctctga | 2340 |
| ctgctcatcc tcatcctcct cggccagcct gccttcctcc ggcaggagca gcctgggcag | 2400 |
| tcaccagctc ccgcgggggt acatctccat tccggtgata cacgagcaga acgttacccg | 2460 |
| gccagcagcc cagccctcct tccaccaagc ccagaagacg cactacccag cgcagcaggg | 2520 |
| ggagtaccag acccaccagc ctgtgtacca caagatccag ggggatgact gggagccccg | 2580 |
| gcccctgcgg gcggcatccc cgttcaggtc atctgtccag ggtgcatcga gccgggaggg | 2640 |
| ctcaccagcc aggagcagca cgccactcca ctccccctcg cccatccgtg tgcacaccgt | 2700 |
| ggtcgacagg cctcagcagc ccatgaccca tcgagaaact gcacctgttt cccagcctga | 2760 |
| aaacaaacca gaaagtaagc caggcccagt tggaccagaa ctccctcctg gacacatccc | 2820 |
| aattcaagtg atccgcaaag aggtggattc taaacctgtt tcccagaagc ccccacctcc | 2880 |
| ctctgagaag gtagaggtga aagttccccc tgctccagtt ccttgtcctc ctcccagccc | 2940 |
| tggcccttct gctgtcccct cttcccccaa gagtgtggct acagaagaga gggcagcccc | 3000 |
| cagcactgcc cctgcagaag ctacacctcc aaaaccagga gaagccgagg ctcccccaaa | 3060 |
| acatccagga gtgctgaaag tggaagccat cctggagaag gtacaggggc tggagcaggc | 3120 |
| tgtagacaac tttgaaggca agaagactga caaaaagtac ctgatgatcg aagagtattt | 3180 |
| gaccaaagag ctgctggccc tggattcagt ggaccccgag ggacgagccg atgtgcgtca | 3240 |
| ggccaggaga gacggtgtca ggaaggttca gaccatcttg gaaaaacttg aacagaaagc | 3300 |
| cattgatgtc ccaggtcaag tccaggtcta tgaactccag cccagcaacc ttgaagcaga | 3360 |
| tcagccactg caggcaatca tggagatggg tgccgtggca gcagacaagg gcaagaaaaa | 3420 |
| tgctggaaat gcagaagatc cccacacaga aacccagcag ccagaagcca cagcagcagc | 3480 |
| gacttcaaac cccagcagca tgacagacac ccctggtaac ccagcagcac cgggatccgg | 3540 |
| cagtggagag ggcagaggaa gtctgctaac atgcggtgac gtcgaggaga atcctggccc | 3600 |
| aATGAGCAAG GGCGAGGAGC TGTTCACCGG CGTGGTGCCC ATCCTGGTGG AGCTGGACGG | 3660 |
| CGACGTGAAC GGCCACAAGT TCAGCGTGAG AGGCGAGGGC GAGGGCGACG CCACCAACGG | 3720 |
| CAAGCTGACC CTGAAGTTCA TCTGCACCAC CGGCAAGCTG CCCGTGCCCT GGCCCACCCT | 3780 |
| GGTGACCACC CTGACCTACG GCGTGCTGTG CTTCAGCAGA TACCCCGACC ACATGAAGAG | 3840 |
| ACACGACTTC TTCAAGAGCG CCATGCCCGA GGGCTACGTG CAGGAGAGAA CCATCAGCTT | 3900 |
| CAAGGACGAC GGCACCTACA AGACCAGAGC CGAGGTGAAG TTCGAGGGCG ACACCCTGGT | 3960 |
| GAACAGAATC GAGCTGAAGG GCATCGACTT CAAGGAGGAC GGCAACATCC TGGGCCACAA | 4020 |
| GCTGGAGTAC AACTTCAACA GCCACAACGT GTACATCACC GCCGACAAGC AGAAGAACGG | 4080 |
| CATCAAGGCC TACTTCAAGA TCAGACACAA CGTGGAGGAC GGCAGCGTGC AGCTGGCCGA | 4140 |
| CCACTACCAG CAGAACACCC CCATCGGCGA CGGCCCCGTG CTGCTGCCCG ACAACCACTA | 4200 |
| CCTGAGCACC CAGAGCGTGC TGAGCAAGGA CCCCAACGAG AAGAGAGACC ACATGGTGCT | 4260 |
| GCTGGAGGAC GTGACCGCCG CCGGCATCAC CCACGGCATG GACGAGCTGT ACAAGTGAtc | 4320 |
| aacctctgga ttacaaaatt tgtgaaagat tgactggtat tcttaactat gttgctcctt | 4380 |
| ttacgctatg tggatacgct gctttaatgc ctttgtatca tgctattgct tcccgtatgg | 4440 |
| ctttcatttt ctcctccttg tataaatcct ggttgctgtc tctttatgag gagttgtggc | 4500 |
| ccgttgtcag gcaacgtggc gtggtgtgca ctgtgtttgc tgacgcaacc cccactggtt | 4560 |
| ggggcattgc caccacctgt cagctccttt ccgggacttt cgctttcccc ctccctattg | 4620 |
| ccacggcgga actcatcgcc gcctgccttg cccgctgctg gacaggggct cggctgttgg | 4680 |
| gcactgacaa ttccgtggtg ttgtcgggga aatcatcgtc ctttccttgg ctgctcgcct | 4740 |
| gtgttgccac ctggattctg cgcgggacgt ccttctgcta cgtcccttcg gccctcaatc | 4800 |
| cagcggacct tccttcccgc ggcctgctgc cggctctgcg gcctcttccg cgtcttcgcc | 4860 |
| ttcgccctca gacgagtcgg atctcccttt gggccgcctc cccgcactgc ccgggtggca | 4920 |
| tccctgtgac ccctccccag tgcctctcct ggccctggaa gttgccactc cagtgcccac | 4980 |
| cagccttgtc ctaataaaat taagttgcat cattttgtct gactaggtgt ccttctataa | 5040 |
| tattatgggg tggagggggg tggtatggag caaggggccc aagttgggaa gaaacctgta | 5100 |
| gggcctgccc taaggaggaa cccctagtga tggagttggc cactccctct ctgcgcgctc | 5160 |
| gctcgctcac tgaggccggg cgaccaaagg tcgcccgacg cccgggcttt gcccgggcgg | 5220 |
| cctcagtgag cgagcgagcg cgcagagagg gagtggccaa | 5260 |

In a certain embodiment, the vector genome comprises, in 5′ to 3′ order, a 5′ ITR; an UBC promoter; a BAG3 transgene; an WPRE(x) element; a Human GH poly(A) signal (hGH) sequence; and a 3′ ITR. The vector genome may comprise, in 5′ to 3′ order, the polynucleotide sequences SEQ ID NO: 114; or polynucleotide sequences sharing 90%, 91%, 92%, 93%, 94%, 95%, 96%, 97%, 98%, 99%, or 100% identity to each of the foregoing. In certain embodiments, this vector genome is packaged in an AAV9 or AAVrh74 vector. In certain embodiments, this vector genome is packaged in an AAVrh74 vector.

SEQ ID NO: 114
ttggccactccctctctgcgcgctcgctcgctcactgaggccgcccgggcaaagcccgggcgtcgggcgaccttt
ggtcgcccggcctcagtgagcgagcgagcgcgcagagagggagtggccaactccatcactaggggttcctcctga
ggacgcgtgatctggcctccgcgccgggttttggcgcctcccgcgggcgcccccctcctcacggcgagcgctgcc
acgtcagacgaagggcgcaggagcgtcctgatccttccgcccggacgctcaggacagcggcccgctgctcataag
actcggccttagaaccccagtatcagcagaaggacattttaggacgggacttgggtgactctagggcactggttt
tctttccagagagcggaacaggcgaggaaaagtagtcccttctcggcgattctgcggagggatctccgtggggcg
gtgaacgccgatgattatataaggacgcgccgggtgtggcacagctagttccgtcgcagccgggatttgggtcgc
ggttcttgtttgtggatcgctgtgatcgtcacttggtgagtagcgggctgctgggctggccggggctttcgtggc
cgccgggccgctcggtgggacggaagcgtgtggagagaccgccaagggctgtagtctgggtccgcgagcaaggtt
gccctgaactgggggttggggggagcgcagcaaaatggcggctgttcccgagtcttgaatggaagacgcttgtga
ggcgggctgtgaggtcgttgaaacaaggtggggggcatggtgggcggcaagaacccaaggtcttgaggccttcgc
taatgcgggaaagctcttattcgggtgagatgggctggggcaccatctggggaccctgacgtgaagtttgtcact
gactggagaactcggtttgtcgtctgttgcgggggcggcagttatgcggtgccgttgggcagtgcacccgtacct
ttgggagcgcgcgccctcgtcgtgtcgtgacgtcacccgttctgttggcttataatgcagggtggggccacctgc
cggtaggtgtgcggtaggcttttctccgtcgcaggacgcagggttcgggcctagggtaggctctcctgaatcgac
aggcgccggacctctggtgaggggagggataagtgaggcgtcagtttctttggtcggttttatgtacctatcttc
ttaagtagctgaagctccggttttgaactatgcgctcggggttggcgagtgtgttttgtgaagttttttaggcac
cttttgaaatgtaatcatttgggtcaatatgtaattttcagtgttagactagtaaattgtccgctaaattctggc
cgtttttggcttttttgttagacgaaggccaccATGagcgccgccacccactcgcccatgatgcaggtggcgtcc
ggcaacggtgaccgcgaccctttgccccccggatgggagatcaagatcgacccgcagaccggctggcccttcttc
gtggaccacaacagccgcaccactacgtggaacgacccgcgcgtgccctctgagggccccaaggagactccatcc
tctgccaatggcccttcccgggagggctctaggctgccgcctgctagggaaggccaccctgtgtacccccagctc
cgaccaggctacattcccattcctgtgctccatgaaggcgctgagaaccggcaggtgcaccctttccatgtctat
ccccagcctgggatgcagcgattccgaactgaggcggcagcagcggctcctcagaggtcccagtcacctctgcgg
ggcatgccagaaaccactcagccagataaacagtgtggacaggtggcagcggcggcggcagcccagcccccagcc
tcccacggacctgagcggtcccagtctccagctgcctctgactgctcatcctcatcctcctcggccagcctgcct
tcctccggcaggagcagcctgggcagtcaccagctcccgcgggggtacatctccattccggtgatacacgagcag
aacgttacccggccagcagcccagccctccttccaccaagcccagaagacgcactacccagcgcagcagggggag
taccagacccaccagcctgtgtaccacaagatccagggggatgactgggagccccggcccctgcgggcggcatcc
ccgttcaggtcatctgtccagggtgcatcgagccgggagggctcaccagccaggagcagcacgccactccactcc
ccctcgcccatccgtgtgcacaccgtggtcgacaggcctcagcagcccatgacccatcgagaaactgcacctgtt
tcccagcctgaaaacaaaccagaaagtaagccaggcccagttggaccagaactccctcctggacacatcccaatt
caagtgatccgcaaagaggtggattctaaacctgtttcccagaagcccccacctccctctgagaaggtagaggtg
aaagttccccctgctccagttccttgtcctcctcccagccctggcccttctgctgtcccctcttcccccaagagt
gtggctacagaagagagggcagcccccagcactgcccctgcagaagctacacctccaaaaccaggagaagccgag
gctcccccaaaacatccaggagtgctgaaagtggaagccatcctggagaaggtacaggggctggagcaggctgta
gacaactttgaaggcaagaagactgacaaaaagtacctgatgatcgaagagtatttgaccaaagagctgctggcc
ctggattcagtggaccccgagggacgagccgatgtgcgtcaggccaggagagacggtgtcaggaaggttcagacc
atcttggaaaaacttgaacagaaagccattgatgtcccaggtcaagtccaggtctatgaactccagcccagcaac
cttgaagcagatcagccactgcaggcaatcatggagatgggtgccgtggcagcagacaagggcaagaaaaatgct
ggaaatgcagaagatccccacacagaaacccagcagccagaagccacagcagcagcgacttcaaaccccagcagc
atgacagacacccctggtaacccagcagcaccgTAGtcaacctctggattacaaaatttgtgaaagattgactgg
tattcttaactatgttgctccttttacgctatgtggatacgctgctttaatgcctttgtatcatgctattgcttc
ccgtatggctttcattttctcctccttgtataaatcctggttgctgtctctttatgaggagttgtggcccgttgt
caggcaacgtggcgtggtgtgcactgtgtttgctgacgcaacccccactggttggggcattgccaccacctgtca
gctcctttccgggactttcgctttccccctccctattgccacggcggaactcatcgccgcctgccttgcccgctg
ctggacaggggctcggctgttgggcactgacaattccgtggtgttgtcggggaaatcatcgtcctttccttggct
gctcgcctgtgttgccacctggattctgcgcgggacgtccttctgctacgtcccttcggccctcaatccagcgga
ccttccttcccgcggcctgctgccggctctgcggcctcttccgcgtcttcgccttcgccctcagacgagtcggat
ctccctttgggccgcctccccgcactgcccgggtggcatccctgtgacccctccccagtgcctctcctggccctg
gaagttgccactccagtgcccaccagccttgtcctaataaaattaagttgcatcattttgtctgactaggtgtcc
ttctataatattatggggtggaggggggtggtatggagcaaggggcccaagttgggaagaaacctgtagggcctg
ccctaaggaggaacccctagtgatggagttggccactccctctctgcgcgctcgctcgctcactgaggccgggcg
accaaaggtcgcccgacgcccgggctttgcccgggcggcctcagtgagcgagcgagcgcgcagagagggagtggc
caa

In each case the optionally WPRE element may be present or absent.

### Adeno-Associated Virus Vector

AAV vectors useful in the practice of the present invention can be packaged into AAV virions (viral particles) using various systems including adenovirus-based and helper-free systems. Standard methods in AAV biology include those described in Kwon and Schaffer. Pharm Res. (2008) 25(3):489-99; Wu et al. Mol. Ther. (2006) 14(3):316-27. Burger et al. Mol. Ther. (2004) 10(2):302-17; Grimm et al. Curr Gene Ther. (2003) 3(4):281-304; Deyle D R, Russell D W. Curr Opin Mol Ther. (2009) 11(4):442-447; McCarty et al. Gene Ther. (2001) 8(16):1248-54; and Duan et al. Mol Ther. (2001) 4(4):383-91. Helper-free systems included those described in U.S. Pat. Nos. 6,004,797; 7,588,772; and 7,094,604;

AAV DNA in the rAAV genomes may be from any AAV variant or serotype for which a recombinant virus can be derived including, but not limited to, AAV variants or serotypes AAV-1, AAV-2, AAV-3, AAV-4, AAV-5, AAV-6, AAV-7, AAV-8, AAV-9, AAV-10, AAV-11, AAV-12, AAV-13, AAVrh.74, and AAVrh10. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promoter efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6kb), rAAV comprising a self-complementary genome can only hold about half of that amount (≈2.4kb).

In other cases, the rAAV vector comprises a single stranded genome. As defined herein, a “single standard” genome refers to a genome that is not self-complementary. In most cases, non-recombinant AAVs have singled stranded DNA genomes. There have been some indications that rAAVs should be scAAVs to achieve efficient transduction of cells. The present disclosure contemplates, however, rAAV vectors that maybe have singled stranded genomes, rather than self-complementary genomes, with the understanding that other genetic modifications of the rAAV vector may be beneficial to obtain optimal gene transcription in target cells.

In some cases, the rAAV vector is of the serotype AAV1, AAV2, AAV4, AAV5, AAV6, AAV7, AAV8, AAV9, AAV10, AAV 11, AAV12, AAV13, AAVrh10, or AAVrh74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). In certain embodiments, the rAAV vector is of serotype AAVrh74. In some cases, the rAAV vector is of the serotype AAV9. In some embodiments, said rAAV vector is of serotype AAV9 and comprises a single stranded genome. In some embodiments, said rAAV vector is of serotype AAV9 and comprises a self-complementary genome. In some embodiments, a rAAV vector comprises the inverted terminal repeat (ITR) sequences of AAV2. In some embodiments, the rAAV vector comprises an AAV2 genome, such that the rAAV vector is an AAV-2/9 vector, an AAV-2/6 vector, or an AAV-2/8 vector.

Full-length sequences and sequences for capsid genes for most known AAVs are provided in U.S. Pat. No. 8,524,446, which is incorporated herein in its entirety.

AAV vectors may comprise wild-type AAV sequence or they may comprise one or more modifications to a wild-type AAV sequence. In certain embodiments, an AAV vector comprises one or more amino acid modifications, optionally substitutions, deletions, or insertions, within a capsid protein, optionally VP1, VP2 and/or VP3. In particular embodiments, the modification provides for reduced immunogenicity when the AAV vector is provided to a subject.

Capsid proteins of a rAAV may be modified so that the rAAV is targeted to a particular target tissue of interest such as cardiomyocytes. In some embodiments, the rAAV is directly injected into the intracerebroventricular space of the subject.

In some embodiments, the rAAV virion is an AAVrh.74 rAAV virion. The capsid many be an AAVrh.74 capsid or functional variant thereof. In some embodiments, the AAVrh.74 capsid shares at least 98%, 99%, or 100% identity to a reference AAVrh.74 capsid, e.g., SEQ ID NO: 100. In particular embodiments, the rAAVrh.74 rAAV virion is preferred for targeting adults with DCM and has greater safety and/or efficacy than other AAV serotypes.

In some embodiments, the rAAV virion is an AAV2 rAAV virion. The capsid many be an AAV2 capsid or functional variant thereof. In some embodiments, the AAV2 capsid shares at least 98%, 99%, or 100% identity to a reference AAV2 capsid, e.g., SEQ ID NO: 96.

SEQ ID NO: 96
Met Ala Ala Asp Gly Tyr Leu Pro Asp Trp Leu Glu Asp Thr Leu Ser
Glu Gly Ile Arg Gln Trp Trp Lys Leu Lys Pro Gly Pro Pro Pro Pro
Lys Pro Ala Glu Arg His Lys Asp Asp Ser Arg Gly Leu Val Leu Pro
Gly Tyr Lys Tyr Leu Gly Pro Phe Asn Gly Leu Asp Lys Gly Glu Pro
Val Asn Glu Ala Asp Ala Ala Ala Leu Glu His Asp Lys Ala Tyr Asp
Arg Gln Leu Asp Ser Gly Asp Asn Pro Tyr Leu Lys Tyr Asn His Ala
Asp Ala Glu Phe Gln Glu Arg Leu Lys Glu Asp Thr Ser Phe Gly Gly
Asn Leu Gly Arg Ala Val Phe Gln Ala Lys Lys Arg Val Leu Glu Pro
Leu Gly Leu Val Glu Glu Pro Val Lys Thr Ala Pro Gly Lys Lys Arg
Pro Val Glu His Ser Pro Val Glu Pro Asp Ser Ser Ser Gly Thr Gly
Lys Ala Gly Gln Gln Pro Ala Arg Lys Arg Leu Asn Phe Gly Gln Thr
Gly Asp Ala Asp Ser Val Pro Asp Pro Gln Pro Leu Gly Gln Pro Pro
Ala Ala Pro Ser Gly Leu Gly Thr Asn Thr Met Ala Thr Gly Ser Gly
Ala Pro Met Ala Asp Asn Asn Glu Gly Ala Asp Gly Val Gly Asn Ser
Ser Gly Asn Trp His Cys Asp Ser Thr Trp Met Gly Asp Arg Val Ile
Thr Thr Ser Thr Arg Thr Trp Ala Leu Pro Thr Tyr Asn Asn His Leu
Tyr Lys Gln Ile Ser Ser Gln Ser Gly Ala Ser Asn Asp Asn His Tyr
Phe Gly Tyr Ser Thr Pro Trp Gly Tyr Phe Asp Phe Asn Arg Phe His
Cys His Phe Ser Pro Arg Asp Trp Gln Arg Leu Ile Asn Asn Asn Trp
Gly Phe Arg Pro Lys Arg Leu Asn Phe Lys Leu Phe Asn Ile Gln Val
Lys Glu Val Thr Gln Asn Asp Gly Thr Thr Thr Ile Ala Asn Asn Leu
Thr Ser Thr Val Gln Val Phe Thr Asp Ser Glu Tyr Gln Leu Pro Tyr
Val Leu Gly Ser Ala His Gln Gly Cys Leu Pro Pro Phe Pro Ala Asn
Val Phe Met Val Pro Gln Tyr Gly Tyr Leu Thr Leu Asn Asn Gly Ser
Gln Ala Val Gly Arg Ser Ser Phe Tyr Cys Leu Glu Tyr Phe Pro Ser
Gln Met Leu Arg Thr Gly Asn Asn Phe Thr Phe Ser Tyr Thr Phe Glu
Asp Val Pro Phe His Ser Ser Tyr Ala His Ser Gln Ser Leu Asp Arg
Leu Met Asn Pro Leu Ile Asp Gln Tyr Leu Tyr Tyr Leu Ser Arg Thr
Asn Thr Pro Ser Gly Thr Thr Thr Gln Ser Arg Leu Gln Phe Ser Gln
Ala Gly Ala Ser Asp Ile Arg Asp Gln Ser Arg Asn Trp Leu Pro Gly
Pro Cys Tyr Arg Gln Gln Arg Val Ser Lys Thr Ser Ala Asp Asn Asn
Asn Ser Glu Tyr Ser Trp Thr Gly Ala Thr Lys Tyr His Leu Asn Gly
Arg Asp Ser Leu Val Asn Pro Gly Pro Ala Met Ala Ser His Lys Asp
Asp Glu Glu Lys Phe Phe Pro Gln Ser Gly Val Leu Ile Phe Gly Lys
Gln Gly Ser Glu Lys Thr Asn Val Asp Ile Glu Lys Val Met Ile Thr
Asp Glu Glu Glu Ile Arg Thr Thr Asn Pro Val Ala Thr Glu Gln Tyr
Gly Ser Val Ser Thr Asn Leu Gln Arg Gly Asn Arg Gln Ala Ala Thr
Ala Asp Val Asn Thr Gln Gly Val Leu Pro Gly Met Val Trp Gln Asp
Arg Asp Val Tyr Leu Gln Gly Pro Ile Trp Ala Lys Ile Pro His Thr
Asp Gly His Phe His Pro Ser Pro Leu Met Gly Gly Phe Gly Leu Lys
His Pro Pro Pro Gln Ile Leu Ile Lys Asn Thr Pro Val Pro Ala Asn
Pro Ser Thr Thr Phe Ser Ala Ala Lys Phe Ala Ser Phe Ile Thr Gln
Tyr Ser Thr Gly Gln Val Ser Val Glu Ile Glu Trp Glu Leu Gln Lys
Glu Asn Ser Lys Arg Trp Asn Pro Glu Ile Gln Tyr Thr Ser Asn Tyr
Asn Lys Ser Val Asn Val Asp Phe Thr Val Asp Thr Asn Gly Val Tyr
Ser Glu Pro Arg Pro Ile Gly Thr Arg Tyr Leu Thr Arg Asn Leu

In some embodiments, the rAAV virion is an AAV9 rAAV virion. The capsid may be an AAV9 capsid or functional variant thereof. In some embodiments, the AAV9 capsid shares at least 98%, 99%, or 100% identity to a reference AAV9 capsid, e.g., SEQ ID NO: 97.

SEQ ID NO: 97
Met Ala Ala Asp Gly Tyr Leu Pro Asp Trp Leu Glu Asp Asn Leu Ser
Glu Gly Ile Arg Glu Trp Trp Ala Leu Lys Pro Gly Ala Pro Gln Pro
Lys Ala Asn Gln Gln His Gln Asp Asn Ala Arg Gly Leu Val Leu Pro
Gly Tyr Lys Tyr Leu Gly Pro Gly Asn Gly Leu Asp Lys Gly Glu Pro
Val Asn Ala Ala Asp Ala Ala Ala Leu Glu His Asp Lys Ala Tyr Asp
Gln Gln Leu Lys Ala Gly Asp Asn Pro Tyr Leu Lys Tyr Asn His Ala
Asp Ala Glu Phe Gln Glu Arg Leu Lys Glu Asp Thr Ser Phe Gly Gly
Asn Leu Gly Arg Ala Val Phe Gln Ala Lys Lys Arg Leu Leu Glu Pro
Leu Gly Leu Val Glu Glu Ala Ala Lys Thr Ala Pro Gly Lys Lys Arg
Pro Val Glu Gln Ser Pro Gln Glu Pro Asp Ser Ser Ala Gly Ile Gly
Lys Ser Gly Ala Gln Pro Ala Lys Lys Arg Leu Asn Phe Gly Gln Thr
Gly Asp Thr Glu Ser Val Pro Asp Pro Gln Pro Ile Gly Glu Pro Pro
Ala Ala Pro Ser Gly Val Gly Ser Leu Thr Met Ala Ser Gly Gly Gly
Ala Pro Val Ala Asp Asn Asn Glu Gly Ala Asp Gly Val Gly Ser Ser
Ser Gly Asn Trp His Cys Asp Ser Gln Trp Leu Gly Asp Arg Val Ile
Thr Thr Ser Thr Arg Thr Trp Ala Leu Pro Thr Tyr Asn Asn His Leu
Tyr Lys Gln Ile Ser Asn Ser Thr Ser Gly Gly Ser Ser Asn Asp Asn
Ala Tyr Phe Gly Tyr Ser Thr Pro Trp Gly Tyr Phe Asp Phe Asn Arg
Phe His Cys His Phe Ser Pro Arg Asp Trp Gln Arg Leu Ile Asn Asn
Asn Trp Gly Phe Arg Pro Lys Arg Leu Asn Phe Lys Leu Phe Asn Ile
Gln Val Lys Glu Val Thr Asp Asn Asn Gly Val Lys Thr Ile Ala Asn
Asn Leu Thr Ser Thr Val Gln Val Phe Thr Asp Ser Asp Tyr Gln Leu
Pro Tyr Val Leu Gly Ser Ala His Glu Gly Cys Leu Pro Pro Phe Pro
Ala Asp Val Phe Met Ile Pro Gln Tyr Gly Tyr Leu Thr Leu Asn Asp
Gly Ser Gln Ala Val Gly Arg Ser Ser Phe Tyr Cys Leu Glu Tyr Phe
Pro Ser Gln Met Leu Arg Thr Gly Asn Asn Phe Gln Phe Ser Tyr Glu
Phe Glu Asn Val Pro Phe His Ser Ser Tyr Ala His Ser Gln Ser Leu
Asp Arg Leu Met Asn Pro Leu Ile Asp Gln Tyr Leu Tyr Tyr Leu Ser
Lys Thr Ile Asn Gly Ser Gly Gln Asn Gln Gln Thr Leu Lys Phe Ser
Val Ala Gly Pro Ser Asn Met Ala Val Gln Gly Arg Asn Tyr Ile Pro
Gly Pro Ser Tyr Arg Gln Gln Arg Val Ser Thr Thr Val Thr Gln Asn
Asn Asn Ser Glu Phe Ala Trp Pro Gly Ala Ser Ser Trp Ala Leu Asn
Gly Arg Asn Ser Leu Met Asn Pro Gly Pro Ala Met Ala Ser His Lys
Glu Gly Glu Asp Arg Phe Phe Pro Leu Ser Gly Ser Leu Ile Phe Gly
Lys Gln Gly Thr Gly Arg Asp Asn Val Asp Ala Asp Lys Val Met Ile
Thr Asn Glu Glu Glu Ile Lys Thr Thr Asn Pro Val Ala Thr Glu Ser
Tyr Gly Gln Val Ala Thr Asn His Gln Ser Ala Gln Ala Gln Ala Gln
Thr Gly Trp Val Gln Asn Gln Gly Ile Leu Pro Gly Met Val Trp Gln
Asp Arg Asp Val Tyr Leu Gln Gly Pro Ile Trp Ala Lys Ile Pro His
Thr Asp Gly Asn Phe His Pro Ser Pro Leu Met Gly Gly Phe Gly Met
Lys His Pro Pro Pro Gln Ile Leu Ile Lys Asn Thr Pro Val Pro Ala
Asp Pro Pro Thr Ala Phe Asn Lys Asp Lys Leu Asn Ser Phe Ile Thr
Gln Tyr Ser Thr Gly Gln Val Ser Val Glu Ile Glu Trp Glu Leu Gln
Lys Glu Asn Ser Lys Arg Trp Asn Pro Glu Ile Gln Tyr Thr Ser Asn
Tyr Tyr Lys Ser Asn Asn Val Glu Phe Ala Val Asn Thr Glu Gly Val
Tyr Ser Glu Pro Arg Pro Ile Gly Thr Arg Tyr Leu Thr Arg Asn Leu

In some embodiments, the rAAV virion is an AAV6 rAAV virion. The capsid may be an AAV6 capsid or functional variant thereof. In some embodiments, the AAV6 capsid shares at least 98%, 99%, or 100% identity to a reference AAV6 capsid, e.g., SEQ ID NO: 98.

SEQ ID NO: 98
Met Ala Ala Asp Gly Tyr Leu Pro Asp Trp Leu Glu Asp Asn Leu Ser
Glu Gly Ile Arg Glu Trp Trp Asp Leu Lys Pro Gly Ala Pro Lys Pro
Lys Ala Asn Gln Gln Lys Gln Asp Asp Gly Arg Gly Leu Val Leu Pro
Gly Tyr Lys Tyr Leu Gly Pro Phe Asn Gly Leu Asp Lys Gly Glu Pro
Val Asn Ala Ala Asp Ala Ala Ala Leu Glu His Asp Lys Ala Tyr Asp
Gln Gln Leu Lys Ala Gly Asp Asn Pro Tyr Leu Arg Tyr Asn His Ala
Asp Ala Glu Phe Gln Glu Arg Leu Gln Glu Asp Thr Ser Phe Gly Gly
Asn Leu Gly Arg Ala Val Phe Gln Ala Lys Lys Arg Val Leu Glu Pro
Phe Gly Leu Val Glu Glu Gly Ala Lys Thr Ala Pro Gly Lys Lys Arg
Pro Val Glu Gln Ser Pro Gln Glu Pro Asp Ser Ser Ser Gly Ile Gly
Lys Thr Gly Gln Gln Pro Ala Lys Lys Arg Leu Asn Phe Gly Gln Thr
Gly Asp Ser Glu Ser Val Pro Asp Pro Gln Pro Leu Gly Glu Pro Pro
Ala Thr Pro Ala Ala Val Gly Pro Thr Thr Met Ala Ser Gly Gly Gly
Ala Pro Met Ala Asp Asn Asn Glu Gly Ala Asp Gly Val Gly Asn Ala
Ser Gly Asn Trp His Cys Asp Ser Thr Trp Leu Gly Asp Arg Val Ile
Thr Thr Ser Thr Arg Thr Trp Ala Leu Pro Thr Tyr Asn Asn His Leu
Tyr Lys Gln Ile Ser Ser Ala Ser Thr Gly Ala Ser Asn Asp Asn His
Tyr Phe Gly Tyr Ser Thr Pro Trp Gly Tyr Phe Asp Phe Asn Arg Phe
His Cys His Phe Ser Pro Arg Asp Trp Gln Arg Leu Ile Asn Asn Asn
Trp Gly Phe Arg Pro Lys Arg Leu Asn Phe Lys Leu Phe Asn Ile Gln
Val Lys Glu Val Thr Thr Asn Asp Gly Val Thr Thr Ile Ala Asn Asn
Leu Thr Ser Thr Val Gln Val Phe Ser Asp Ser Glu Tyr Gln Leu Pro
Tyr Val Leu Gly Ser Ala His Gln Gly Cys Leu Pro Pro Phe Pro Ala
Asp Val Phe Met Ile Pro Gln Tyr Gly Tyr Leu Thr Leu Asn Asn Gly
Ser Gln Ala Val Gly Arg Ser Ser Phe Tyr Cys Leu Glu Tyr Phe Pro
Ser Gln Met Leu Arg Thr Gly Asn Asn Phe Thr Phe Ser Tyr Thr Phe
Glu Asp Val Pro Phe His Ser Ser Tyr Ala His Ser Gln Ser Leu Asp
Arg Leu Met Asn Pro Leu Ile Asp Gln Tyr Leu Tyr Tyr Leu Asn Arg
Thr Gln Asn Gln Ser Gly Ser Ala Gln Asn Lys Asp Leu Leu Phe Ser
Arg Gly Ser Pro Ala Gly Met Ser Val Gln Pro Lys Asn Trp Leu Pro
Gly Pro Cys Tyr Arg Gln Gln Arg Val Ser Lys Thr Lys Thr Asp Asn
Asn Asn Ser Asn Phe Thr Trp Thr Gly Ala Ser Lys Tyr Asn Leu Asn
Gly Arg Glu Ser Ile Ile Asn Pro Gly Thr Ala Met Ala Ser His Lys
Asp Asp Lys Asp Lys Phe Phe Pro Met Ser Gly Val Met Ile Phe Gly
Lys Glu Ser Ala Gly Ala Ser Asn Thr Ala Leu Asp Asn Val Met Ile
Thr Asp Glu Glu Glu Ile Lys Ala Thr Asn Pro Val Ala Thr Glu Arg
Phe Gly Thr Val Ala Val Asn Leu Gln Ser Ser Ser Thr Asp Pro Ala
Thr Gly Asp Val His Val Met Gly Ala Leu Pro Gly Met Val Trp Gln
Asp Arg Asp Val Tyr Leu Gln Gly Pro Ile Trp Ala Lys Ile Pro His
Thr Asp Gly His Phe His Pro Ser Pro Leu Met Gly Gly Phe Gly Leu
Lys His Pro Pro Pro Gln Ile Leu Ile Lys Asn Thr Pro Val Pro Ala
Asn Pro Pro Ala Glu Phe Ser Ala Thr Lys Phe Ala Ser Phe Ile Thr
Gln Tyr Ser Thr Gly Gln Val Ser Val Glu Ile Glu Trp Glu Leu Gln
Lys Glu Asn Ser Lys Arg Trp Asn Pro Glu Val Gln Tyr Thr Ser Asn
Tyr Ala Lys Ser Ala Asn Val Asp Phe Thr Val Asp Asn Asn Gly Leu
Tyr Thr Glu Pro Arg Pro Ile Gly Thr Arg Tyr Leu Thr Arg Pro Leu

In some embodiments, the rAAV virion is an AAVrh.10 rAAV virion. The capsid may be an AAVrh.10 capsid or functional variant thereof. In some embodiments, the AAVrh.10 capsid shares at least 98%, 99%, or 100% identity to a reference AAVrh.10 capsid, e.g., SEQ ID NO: 99.

SEQ ID NO: 99
Met Ala Ala Asp Gly Tyr Leu Pro Asp Trp Leu Glu Asp Asn Leu Ser
Glu Gly Ile Arg Glu Trp Trp Asp Leu Lys Pro Gly Ala Pro Lys Pro
Lys Ala Asn Gln Gln Lys Gln Asp Asp Gly Arg Gly Leu Val Leu Pro
Gly Tyr Lys Tyr Leu Gly Pro Phe Asn Gly Leu Asp Lys Gly Glu Pro
Val Asn Ala Ala Asp Ala Ala Ala Leu Glu His Asp Lys Ala Tyr Asp
Gln Gln Leu Lys Ala Gly Asp Asn Pro Tyr Leu Arg Tyr Asn His Ala
Asp Ala Glu Phe Gln Glu Arg Leu Gln Glu Asp Thr Ser Phe Gly Gly
Asn Leu Gly Arg Ala Val Phe Gln Ala Lys Lys Arg Val Leu Glu Pro
Leu Gly Leu Val Glu Glu Gly Ala Lys Thr Ala Pro Gly Lys Lys Arg
Pro Val Glu Pro Ser Pro Gln Arg Ser Pro Asp Ser Ser Thr Gly Ile
Gly Lys Lys Gly Gln Gln Pro Ala Lys Lys Arg Leu Asn Phe Gly Gln
Thr Gly Asp Ser Glu Ser Val Pro Asp Pro Gln Pro Ile Gly Glu Pro
Pro Ala Gly Pro Ser Gly Leu Gly Ser Gly Thr Met Ala Ala Gly Gly
Gly Ala Pro Met Ala Asp Asn Asn Glu Gly Ala Asp Gly Val Gly Ser
Ser Ser Gly Asn Trp His Cys Asp Ser Thr Trp Leu Gly Asp Arg Val
Ile Thr Thr Ser Thr Arg Thr Trp Ala Leu Pro Thr Tyr Asn Asn His
Leu Tyr Lys Gln Ile Ser Asn Gly Thr Ser Gly Gly Ser Thr Asn Asp
Asn Thr Tyr Phe Gly Tyr Ser Thr Pro Trp Gly Tyr Phe Asp Phe Asn
Arg Phe His Cys His Phe Ser Pro Arg Asp Trp Gln Arg Leu Ile Asn
Asn Asn Trp Gly Phe Arg Pro Lys Arg Leu Asn Phe Lys Leu Phe Asn
Ile Gln Val Lys Glu Val Thr Gln Asn Glu Gly Thr Lys Thr Ile Ala
Asn Asn Leu Thr Ser Thr Ile Gln Val Phe Thr Asp Ser Glu Tyr Gln
Leu Pro Tyr Val Leu Gly Ser Ala His Gln Gly Cys Leu Pro Pro Phe
Pro Ala Asp Val Phe Met Ile Pro Gln Tyr Gly Tyr Leu Thr Leu Asn
Asn Gly Ser Gln Ala Val Gly Arg Ser Ser Phe Tyr Cys Leu Glu Tyr
Phe Pro Ser Gln Met Leu Arg Thr Gly Asn Asn Phe Glu Phe Ser Tyr
Gln Phe Glu Asp Val Pro Phe His Ser Ser Tyr Ala His Ser Gln Ser
Leu Asp Arg Leu Met Asn Pro Leu Ile Asp Gln Tyr Leu Tyr Tyr Leu
Ser Arg Thr Gln Ser Thr Gly Gly Thr Ala Gly Thr Gln Gln Leu Leu
Phe Ser Gln Ala Gly Pro Asn Asn Met Ser Ala Gln Ala Lys Asn Trp
Leu Pro Gly Pro Cys Tyr Arg Gln Gln Arg Val Ser Thr Thr Leu Ser
Gln Asn Asn Asn Ser Asn Phe Ala Trp Thr Gly Ala Thr Lys Tyr His
Leu Asn Gly Arg Asp Ser Leu Val Asn Pro Gly Val Ala Met Ala Thr
His Lys Asp Asp Glu Glu Arg Phe Phe Pro Ser Ser Gly Val Leu Met
Phe Gly Lys Gln Gly Ala Gly Lys Asp Asn Val Asp Tyr Ser Ser Val
Met Leu Thr Ser Glu Glu Glu Ile Lys Thr Thr Asn Pro Val Ala Thr
Glu Gln Tyr Gly Val Val Ala Asp Asn Leu Gln Gln Gln Asn Ala Ala
Pro Ile Val Gly Ala Val Asn Ser Gln Gly Ala Leu Pro Gly Met Val
Trp Gln Asn Arg Asp Val Tyr Leu Gln Gly Pro Ile Trp Ala Lys Ile
Pro His Thr Asp Gly Asn Phe His Pro Ser Pro Leu Met Gly Gly Phe
Gly Leu Lys His Pro Pro Pro Gln Ile Leu Ile Lys Asn Thr Pro Val
Pro Ala Asp Pro Pro Thr Thr Phe Ser Gln Ala Lys Leu Ala Ser Phe
Ile Thr Gln Tyr Ser Thr Gly Gln Val Ser Val Glu Ile Glu Trp Glu
Leu Gln Lys Glu Asn Ser Lys Arg Trp Asn Pro Glu Ile Gln Tyr Thr
Ser Asn Tyr Tyr Lys Ser Thr Asn Val Asp Phe Ala Val Asn Thr Asp
Gly Thr Tyr Ser Glu Pro Arg Pro Ile Gly Thr Arg Tyr Leu Thr Arg
Asn Leu

In some embodiments, the capsid protein is encoded by a polynucleotide supplied on a plasmid in trans to the transfer plasmid. The polynucleotide sequence of wild-type AAVrh74 cap is provided as SEQ ID NO: 100.

| SEQ ID NO: 100 | |
|---|---|
| atggctgccg atggttatct tccagattgg ctcgaggaca acctctctga gggcattcgc | 6 |
| gagtggtggg acctgaaacc tggagccccg aaacccaaag ccaaccagca aaagcaggac | 120 |
| aacggccggg gtctggtgct tcctggctac aagtacctcg gacccttcaa cggactcgac | 180 |
| aagggggagc ccgtcaacgc ggcggacgca gcggccctcg agcacgacaa ggcctacgac | 240 |
| cagcagctcc aagcgggtga caatccgtac ctgcggtata atcacgccga cgccgagttt | 300 |
| caggagcgtc tgcaagaaga tacgtctttt gggggcaacc tcgggcgcgc agtcttccag | 360 |
| gccaaaaagc gggttctcga acctctgggc ctggttgaat cgccggttaa gacggctcct | 420 |
| ggaaagaaga gaccggtaga gccatcaccc cagcgctctc cagactcctc tacgggcatc | 480 |
| ggcaagaaag gccagcagcc cgcaaaaaag agactcaatt ttgggcagac tggcgactca | 540 |
| gagtcagtcc ccgaccctca accaatcgga gaaccaccag caggcccctc tggtctggga | 600 |
| tctggtacaa tggctgcagg cggtggcgct ccaatggcag acaataacga aggcgccgac | 660 |
| ggagtgggta gttcctcagg aaattggcat tgcgattcca catggctggg cgacagagtc | 720 |
| atcaccacca gcacccgcac ctgggccctg cccacctaca acaaccacct ctacaagcaa | 780 |
| atctccaacg ggacctcggg aggaagcacc aacgacaaca cctacttcgg ctacagcacc | 840 |
| ccctgggggt attttgactt caacagattc cactgccact tttcaccacg tgactggcag | 900 |
| cgactcatca acaacaactg gggattccgg cccaagaggc tcaacttcaa gctcttcaac | 960 |
| atccaagtca aggaggtcac gcagaatgaa ggcaccaaga ccatcgccaa taaccttacc | 1020 |
| agcacgattc aggtctttac ggactcggaa taccagctcc cgtacgtgct cggctcggcg | 1080 |
| caccagggct gcctgcctcc gttcccggcg gacgtcttca tgattcctca gtacgggtac | 1140 |
| ctgactctga acaatggcag tcaggctgtg ggccggtcgt ccttctactg cctggagtac | 1200 |
| tttccttctc aaatgctgag aacgggcaac aactttgaat tcagctacaa cttcgaggac | 1260 |
| gtgcccttcc acagcagcta cgcgcacagc cagagcctgg accggctgat gaaccctctc | 1320 |
| atcgaccagt acttgtacta cctgtcccgg actcaaagca cgggcggtac tgcaggaact | 1380 |
| cagcagttgc tattttctca ggccgggcct aacaacatgt cggctcaggc caagaactgg | 1440 |
| ctacccggtc cctgctaccg gcagcaacgc gtctccacga cactgtcgca gaacaacaac | 1500 |
| agcaactttg cctggacggg tgccaccaag tatcatctga atggcagaga ctctctggtg | 1560 |
| aatcctggcg ttgccatggc tacccacaag gacgacgaag agcgattttt tccatccagc | 1620 |
| ggagtcttaa tgtttgggaa acagggagct ggaaaagaca acgtggacta tagcagcgtg | 1680 |
| atgctaacca gcgaggaaga aataaagacc accaacccag tggccacaga acagtacggc | 1740 |
| gtggtggccg ataacctgca acagcaaaac gccgctccta ttgtaggggc cgtcaatagt | 1800 |
| caaggagcct tacctggcat ggtgtggcag aaccgggacg tgtacctgca gggtcccatc | 1860 |
| tgggccaaga ttcctcatac ggacggcaac tttcatccct cgccgctgat gggaggcttt | 1920 |
| ggactgaagc atccgcctcc tcagatcctg attaaaaaca cacctgttcc cgcggatcct | 1980 |
| ccgaccacct tcaatcaggc caagctggct tctttcatca cgcagtacag taccggccag | 2040 |
| gtcagcgtgg agatcgagtg ggagctgcag aaggagaaca gcaaacgctg gaacccagag | 2100 |
| attcagtaca cttccaacta ctacaaatct acaaatgtgg actttgctgt caatactgag | 2160 |
| ggtacttatt ccgagcctcg ccccattggc acccgttacc tcacccgtaa tctgtaa | 2217 |

The disclosure further provides protein sequences for AAVrh74 VP1, VP2, and VP3, including SEQ ID NOs: 101-103, and homologs or functional variants thereof.

SEQ ID NO: 101
MAADGYLPDWLEDNLSEGIREWWDLKPGAPKPKANQQKQDNGRGLVLPGYKYLGPFNGLDKGEPVNAADAAALEH
DKAYDQQLQAGDNPYLRYNHADAEFQERLQEDTSFGGNLGRAVFQAKKRVLEPLGLVESPVKTAPGKKRPVEPSP
QRSPDSSTGIGKKGQQPAKKRLNFGQTGDSESVPDPQPIGEPPAGPSGLGSGTMAAGGGAPMADNNEGADGVGSS
SGNWHCDSTWLGDRVITTSTRTWALPTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSEYQLPYVLGSAHQGCLPPFPADVFMI
PQYGYLTLNNGSQAVGRSSFYCLEYFPSQMLRTGNNFEFSYNFEDVPFHSSYAHSQSLDRLMNPLIDQYLYYLSR
TQSTGGTAGTQQLLFSQAGPNNMSAQAKNWLPGPCYRQQRVSTTLSQNNNSNFAWTGATKYHLNGRDSLVNPGVA
MATHKDDEERFFPSSGVLMFGKQGAGKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQILIKNTPVPADPPTTFNQAKLASFITQ
YSTGQVSVEIEWELQKENSKRWNPEIQYTSNYYKSTNVDFAVNTEGTYSEPRPIGTRYLTRNL
SEQ ID NO: 102
TAPGKKRPVEPSPQRSPDSSTGIGKKGQQPAKKRLNFGQTGDSESVPDPQPIGEPPAGPSGLGSGTMAAGGGAPM
ADNNEGADGVGSSSGNWHCDSTWLGDRVITTSTRTWALPTYNNHLYKQISNGTSGGSTNDNTYFGYSTPWGYFDF
NRFHCHFSPRDWQRLINNNWGFRPKRLNFKLFNIQVKEVTQNEGTKTIANNLTSTIQVFTDSEYQLPYVLGSAHQ
GCLPPFPADVFMIPQYGYLTLNNGSQAVGRSSFYCLEYFPSQMLRTGNNFEFSYNFEDVPFHSSYAHSQSLDRLM
NPLIDQYLYYLSRTQSTGGTAGTQQLLFSQAGPNNMSAQAKNWLPGPCYRQQRVSTTLSQNNNSNFAWTGATKYH
LNGRDSLVNPGVAMATHKDDEERFFPSSGVLMFGKQGAGKDNVDYSSVMLTSEEEIKTTNPVATEQYGVVADNLQ
QQNAAPIVGAVNSQGALPGMVWQNRDVYLQGPIWAKIPHTDGNFHPSPLMGGFGLKHPPPQILIKNTPVPADPPT
TFNQAKLASFITQYSTGQVSVEIEWELQKENSKRWNPEIQYTSNYYKSTNVDFAVNTEGTYSEPRPIGTRYLTRL
SEQ ID NO: 103
MAAGGGAPMADNNEGADGVGSSSGNWHCDSTWLGDRVITTSTRTWALPTYNNHLYKQISNGTSGGSTNDNTYFGY
STPWGYFDFNRFHCHFSPRDWQRLINNNWGFRPKRLNFKLFNIQVKEVTQNEGTKTIANNLTSTIQVFTDSEYQL
PYVLGSAHQGCLPPFPADVFMIPQYGYLTLNNGSQAVGRSSFYCLEYFPSQMLRTGNNFEFSYNFEDVPFHSSYA
HSQSLDRLMNPLIDQYLYYLSRTQSTGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKDNVDYSSVMLTSEEEIKTTNPVATEQ
YGVVADNLQQQNAAPIVGAVNSQGALPGMVWQNRDVYLQGPIWAKIPHTDGNFHPSPLMGGFGLKHPPPQILIKN
TPVPADPPTTFNQAKLASFITQYSTGQVSVEIEWELQKENSKRWNPEIQYTSNYYKSTNVDFAVNTEGTYSEPRP
IGTRYLTRNL

In certain cases, the AAVrh74 capsid comprises the amino acid sequence set forth in SEQ ID NO: 101. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP1 which is set forth in SEQ ID NO: 101. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP2 which is set forth in SEQ ID NO: 102. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP3 which is set forth in SEQ ID NO: 103.

In some embodiments, the rAAV virion is an AAV-PHP.B rAAV virion or a neutrotrophic variant thereof, such as, without limitation, those disclosed in Int'l Pat. Pub. Nos. WO 2015/038958 A1 and WO 2017/100671 A1. For example, the AAV capsid may comprise at least 4 contiguous amino acids from the sequence TLAVPFK (SEQ ID NO: 105) or KFPVALT (SEQ ID NO: 106), e.g., inserted between a sequence encoding for amino acids 588 and 589 of AAV9.

The capsid many be an AAV-PHP.B capsid or functional variant thereof. In some embodiments, the AAV-PHP.B capsid shares at least 98%, 99%, or 100% identity to a reference AAV-PHP.B capsid, e.g., SEQ ID NO: 104.

SEQ ID NO: 104
Met Ala Ala Asp Gly Tyr Leu Pro Asp Trp Leu Glu Asp Asn Leu Ser
Glu Gly Ile Arg Glu Trp Trp Ala Leu Lys Pro Gly Ala Pro Gln Pro
Lys Ala Asn Gln Gln His Gln Asp Asn Ala Arg Gly Leu Val Leu Pro
Gly Tyr Lys Tyr Leu Gly Pro Gly Asn Gly Leu Asp Lys Gly Glu Pro
Val Asn Ala Ala Asp Ala Ala Ala Leu Glu His Asp Lys Ala Tyr Asp
Gln Gln Leu Lys Ala Gly Asp Asn Pro Tyr Leu Lys Tyr Asn His Ala
Asp Ala Glu Phe Gln Glu Arg Leu Lys Glu Asp Thr Ser Phe Gly Gly
Asn Leu Gly Arg Ala Val Phe Gln Ala Lys Lys Arg Leu Leu Glu Pro
Leu Gly Leu Val Glu Glu Ala Ala Lys Thr Ala Pro Gly Lys Lys Arg
Pro Val Glu Gln Ser Pro Gln Glu Pro Asp Ser Ser Ala Gly Ile Gly
Lys Ser Gly Ala Gln Pro Ala Lys Lys Arg Leu Asn Phe Gly Gln Thr
Gly Asp Thr Glu Ser Val Pro Asp Pro Gln Pro Ile Gly Glu Pro Pro
Ala Ala Pro Ser Gly Val Gly Ser Leu Thr Met Ala Ser Gly Gly Gly
Ala Pro Val Ala Asp Asn Asn Glu Gly Ala Asp Gly Val Gly Ser Ser
Ser Gly Asn Trp His Cys Asp Ser Gln Trp Leu Gly Asp Arg Val Ile
Thr Thr Ser Thr Arg Thr Trp Ala Leu Pro Thr Tyr Asn Asn His Leu
Tyr Lys Gln Ile Ser Asn Ser Thr Ser Gly Gly Ser Ser Asn Asp Asn
Ala Tyr Phe Gly Tyr Ser Thr Pro Trp Gly Tyr Phe Asp Phe Asn Arg
Phe His Cys His Phe Ser Pro Arg Asp Trp Gln Arg Leu Ile Asn Asn
Asn Trp Gly Phe Arg Pro Lys Arg Leu Asn Phe Lys Leu Phe Asn Ile
Gln Val Lys Glu Val Thr Asp Asn Asn Gly Val Lys Thr Ile Ala Asn
Asn Leu Thr Ser Thr Val Gln Val Phe Thr Asp Ser Asp Tyr Gln Leu
Pro Tyr Val Leu Gly Ser Ala His Glu Gly Cys Leu Pro Pro Phe Pro
Ala Asp Val Phe Met Ile Pro Gln Tyr Gly Tyr Leu Thr Leu Asn Asp
Gly Ser Gln Ala Val Gly Arg Ser Ser Phe Tyr Cys Leu Glu Tyr Phe
Pro Ser Gln Met Leu Arg Thr Gly Asn Asn Phe Gln Phe Ser Tyr Glu
Phe Glu Asn Val Pro Phe His Ser Ser Tyr Ala His Ser Gln Ser Leu
Asp Arg Leu Met Asn Pro Leu Ile Asp Gln Tyr Leu Tyr Tyr Leu Ser
Arg Thr Ile Asn Gly Ser Gly Gln Asn Gln Gln Thr Leu Lys Phe Ser
Val Ala Gly Pro Ser Asn Met Ala Val Gln Gly Arg Asn Tyr Ile Pro
Gly Pro Ser Tyr Arg Gln Gln Arg Val Ser Thr Thr Val Thr Gln Asn
Asn Asn Ser Glu Phe Ala Trp Pro Gly Ala Ser Ser Trp Ala Leu Asn
Gly Arg Asn Ser Leu Met Asn Pro Gly Pro Ala Met Ala Ser His Lys
Glu Gly Glu Asp Arg Phe Phe Pro Leu Ser Gly Ser Leu Ile Phe Gly
Lys Gln Gly Thr Gly Arg Asp Asn Val Asp Ala Asp Lys Val Met Ile
Thr Asn Glu Glu Glu Ile Lys Thr Thr Asn Pro Val Ala Thr Glu Ser
Tyr Gly Gln Val Ala Thr Asn His Gln Ser Ala Gln Thr Leu Ala Val
Pro Phe Lys Ala Gln Ala Gln Thr Gly Trp Val Gln Asn Gln Gly Ile
Leu Pro Gly Met Val Trp Gln Asp Arg Asp Val Tyr Leu Gln Gly Pro
Ile Trp Ala Lys Ile Pro His Thr Asp Gly Asn Phe His Pro Ser Pro
Leu Met Gly Gly Phe Gly Met Lys His Pro Pro Pro Gln Ile Leu Ile
Lys Asn Thr Pro Val Pro Ala Asp Pro Pro Thr Ala Phe Asn Lys Asp
Lys Leu Asn Ser Phe Ile Thr Gln Tyr Ser Thr Gly Gln Val Ser Val
Glu Ile Glu Trp Glu Leu Gln Lys Glu Asn Ser Lys Arg Trp Asn Pro
Glu Ile Gln Tyr Thr Ser Asn Tyr Tyr Lys Ser Asn Asn Val Glu Phe
Ala Val Asn Thr Glu Gly Val Tyr Ser Glu Pro Arg Pro Ile Gly Thr
Arg Tyr Leu Thr Arg Asn Leu

Further AAV capsids used in the rAAV virions of the disclosure include those disclosed in Pat. Pub. Nos. WO 2009/012176 A2 and WO 2015/168666 A2.

Without being bound by theory, the present inventors have determined that an AAVrh.74, AAV9 vector, or an AAVrh.10 vector will confer desirable cardiac tropism on the vector. Without being bound by theory, the present inventors have further determined that an AAVrh.74, AAV9 vector, or an AAVrh.10 vector may provide desired specificity to cardiac cells.

In an aspect, the disclosure provides pharmaceutical compositions comprising the rAAV virion of the disclosure and one or more pharmaceutically acceptable carriers, diluents, or excipients.

For purposes of administration, optionally by injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Poloxamer 188, e.g., at 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the certain methods of preparation are vacuum drying and the freeze-drying technique that yield a powder of the active ingredient plus any additional desired ingredient from the previously sterile-filtered solution thereof.

In another aspect, the disclosure comprises a kit comprising an rAAV virion of the disclosure and instructions for use.

In an aspect, the disclosure provides a method of increasing BAG3 activity in a cell, comprising contacting the cell with an rAAV of the disclosure. In another aspect, the disclosure provides a method of increasing BAG3 activity in a subject, comprising administering to the subject an rAAV of the disclosure. In some embodiments, the cell and/or subject is deficient in BAG3 messenger RNA or BAG3 protein expression levels and/or activity and/or comprises a loss-of-function mutation in BAG3. In some embodiments, the cell and/or subject is deficient in BAG3 messenger RNA or BAG3 protein expression levels and/or activity. The cell may be a cardiac cell, e.g. a cardiomyocyte cell. In particular embodiments, the subject is a mammal, e.g., a human.

In some embodiments, the method promotes survival of cardiac cell, e.g. a cardiomyocyte cell, in cell culture and/or in vivo. In some embodiments, the method promotes and/or restores function of the heart.

In another aspect, the disclosure provides a method of treating a disease or disorder in a subject in need thereof, comprising administering to the subject an effective amount of an rAAV virion of the disclosure. In some embodiments, the disease or disorder is a cardiac disease or disorder. Illustrative cardiac disorders include heart failure, dilated cardiomyopathy (DCM), such as such as BAG3-related dilated cardiomyopathy, BAG3-related myofibrillar myopathy, familial isolated dilated cardiomyopathy, or cardiomyopathy, dilated, 1hh (CMD1HH), hypertrophic cardiomyopathy, atrial fibrillation, arrhythmia, sinus node disease, hypertensive heart disease, cardiac hypertrophy, atrial fibrosis, myocardial infarction, symptomatic sick sinus syndrome, atrial disease, and myocardial infarction. In certain embodiments, the subject suffers from or is at risk for DCM. In particular embodiments, the subject has a cardiomyopathy e.g., dilated cardiomyopathy (DCM), such as BAG3-related dilated cardiomyopathy, BAG3-related myofibrillar myopathy, familial isolated dilated cardiomyopathy, or cardiomyopathy, dilated, 1hh (CMD1HH). In particular embodiments, the subject is a mammal, e.g., a human, having a loss-of-function mutation in a BAG3 gene. In particular embodiments, the subject is a mammal, e.g., a human, having a mutation in BAG3; e.g., E455K. In particular methods, treatment with the rAAV virion results in expression of the BAG3 protein encoded by the rAAV virion in the subject, e.g., in the subject's heart or cardiac tissue. In certain embodiments, treatment with the rAAV virion results in at least two-fold, at least five-fold, at least ten-fold, or more BAG3 protein levels detectable in the subject's heart.

The AAV-mediated delivery of BAG3 protein to the heart may increase life span, prevent or attenuate cardiac cell degeneration, heart failure, scarring, reduced ejection fraction, arrythmia, angina, exercise intolerance, angina (chest pain), sudden cardiac death, exertional myalgias and cramps. The AAV-mediated delivery of BAG3 protein to the heart may show improvement from, or prevent normal disease course detected by use of echocardiography, pathological electrocardiogram, cardiac MRI, heart biopsy, decrease in paroxysmal ventricular arrhythmias, and/or decrease in sudden cardiac death.

The methods disclosed herein may provide efficient biodistribution in the heart. They may result in sustained expression in all, or a substantial fraction of, cardiac cells, e.g., cardiomyocytes. Notably, the methods disclosed herein may provide long-lasting expression of BAG3 protein throughout the life of the subject following AAV vector administration. In some embodiments, BAG3 protein expression in response to treatment lasts at least 1, 2, 3, 4, 5, 6, 7, 8, 9, 10, 15, 20, 25, 30, 35, or 40 years.

Combination therapies are also contemplated by the invention. Combinations of methods of the invention with standard medical treatments (e.g., corticosteroids or topical pressure reducing medications) are specifically contemplated, as are combinations with novel therapies. In some cases, a subject may be treated with a steroid and/or combination of immune suppressing agents to prevent or to reduce an immune response to administration of a rAAV described herein.

In some embodiments, the AAV vector (e.g., AAV9, AAVrh.74, or AAVrh.10 vector) is administered at a dose of between about 1×10¹² and 5×10¹⁴ vector genomes (vg) or between about 1×10¹² and 6×10¹⁴ vg of the AAV vector per kilogram (vg) of total body mass of the subject (vg/kg). In some embodiments, the AAV vector is administered at a dose of between about 1×10¹³ and 5×10¹⁴ vg/kg. In some embodiments, the AAV vector is administered at a dose of between about 5×10¹³ and 3×10¹⁴ vg/kg. In some embodiments, the AAV vector is administered at a dose of between about 5×10¹³ and 1×10¹⁴ vg/kg. In some embodiments, the AAV vector is administered at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg. In certain embodiments, the AAV vector delivered at any of these doses is an AAV9 vector or an AAV rh74 vector. In some cases, it may be advantageous to use a higher dose for an AAV rh74 vector than for an AAV9 vector. In some embodiments, the AAV vector, e.g., an AVrh.74 vector, is administered at a dosage of at least about 5×10¹³ vg/kg, at least about 6×10¹³ vg/kg, at least about 7×10¹³ vg/kg, at least about 8×10¹³ vg/kg, at least about 9×10¹³ vg/kg, at least about 1×10¹⁴ vg/kg, at least about 2×10¹⁴ vg/kg, at least about 3×10¹⁴ vg/kg, at least about 4×10¹⁴ vg/kg, at least about 5×10¹⁴ vg/kg, at least about 6×10¹⁴ vg/kg, or at least about 7×10¹⁴ vg/kg.

In some embodiments, the AAV vector (e.g., AAV9, AAVrh.74, or AAVrh.10 vector) is administered at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg. In certain embodiments, the AAV vector delivered at any of these doses is an AAV9 vector or an AAV rh74 vector.

In some embodiments, the AAV vector (e.g., AAV9, AAVrh.74, or AAVrh.10 vector) is administered at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg. In certain embodiments, the AAV vector delivered at any of these doses is an AAV9 vector or an AAV rh74 vector.

In some embodiments, the AAV vector (e.g., AAV9, AAVrh.74, or AAVrh.10 vector) is administered systemically at a dose of between about 1×10¹² and 5×10¹⁴ vector genomes (vg) of the AAV vector per kilogram (vg) of total body mass of the subject (vg/kg). In some embodiments, the AAV vector is administered systemically at a dose of between about 1×10¹³ and 5×10¹⁴ vg/kg. In some embodiments, the AAV vector is administered systemically at a dose of between about 5×10¹³ and 3×10¹⁴ vg/kg. In some embodiments, the AAV vector is administered systemically at a dose of between about 5×10¹³ and 1×10¹⁴ vg/kg. In some embodiments, the AAV vector is administered systemically at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg. In some embodiments, the AAV vector, e.g., an AVrh.74 vector, is administered at a dosage of at least about 5×10¹³ vg/kg, at least about 6×10¹³ vg/kg, at least about 7×10¹³ vg/kg, at least about 8×10¹³ vg/kg, at least about 9×10¹³ vg/kg, at least about 1×10¹⁴ vg/kg, at least about 2×10¹⁴ vg/kg, at least about 3×10¹⁴ vg/kg, at least about 4×10¹⁴ vg/kg, at least about 5×10¹⁴ vg/kg, at least about 6×10¹⁴ vg/kg, or at least about 7×10¹⁴ vg/kg. In certain embodiments, the AAV vector delivered at any of these doses is an AAV9 vector or an AAV rh74 vector.

In some embodiments, the AAV vector (e.g., AAV9, AAVrh.74, or AAVrh.10 vector) is administered systemically at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹⁵ vg/kg, about 3×10¹⁵ vg/kg, about 5×10¹⁵ vg/kg, or about 7×10¹⁵ vg/kg. In certain embodiments, the AAV vector delivered at any of these doses is an AAV9 vector or an AAV rh74 vector.

In some embodiments, the AAV vector (e.g., AAV9, AAVrh.74, or AAVrh.10 vector) is administered systemically at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10⁵ vg/kg, 5×10¹⁵ vg/kg, or 7×10¹⁵ vg/kg. In certain embodiments, the AAV vector delivered at any of these doses is an AAV9 vector or an AAV rh74 vector.

In some embodiments, the AAV vector (e.g., AAV9, AAVrh.74, or AAVrh.10 vector) is administered intravenously at a dose of between about 1×10¹² and 5×10¹⁴ vector genomes (vg) of the AAV vector per kilogram (vg) of total body mass of the subject (vg/kg). In some embodiments, the AAV vector is administered intravenously at a dose of between about 1×10¹³ and 5×10¹⁴ vg/kg. In some embodiments, the AAV vector is administered intravenously at a dose of between about 5×10¹³ and 3×10¹⁴ vg/kg. In some embodiments, the AAV vector is administered intravenously at a dose of between about 5×10¹³ and 1×10¹⁴ vg/kg. In some embodiments, the AAV vector is administered intravenously at a dose of less than about 1×10¹² vg/kg, less than about 3×10¹² vg/kg, less than about 5×10¹² vg/kg, less than about 7×10¹² vg/kg, less than about 1×10¹³ vg/kg, less than about 3×10¹³ vg/kg, less than about 5×10¹³ vg/kg, less than about 7×10¹³ vg/kg, less than about 1×10¹⁴ vg/kg, less than about 3×10¹⁴ vg/kg, less than about 5×10¹⁴ vg/kg, less than about 7×10¹⁴ vg/kg, less than about 1×10¹⁵ vg/kg, less than about 3×10¹⁵ vg/kg, less than about 5×10¹⁵ vg/kg, or less than about 7×10¹⁵ vg/kg. In some embodiments, the AAV vector, e.g., an AVrh.74 vector, is administered at a dosage of at least about 5×10¹³ vg/kg, at least about 6×10¹³ vg/kg, at least about 7×10¹³ vg/kg, at least about 8×10¹³ vg/kg, at least about 9×10¹³ vg/kg, at least about 1×10¹⁴ vg/kg, at least about 2×10¹⁴ vg/kg, at least about 3×10¹⁴ vg/kg, at least about 4×10¹⁴ vg/kg, at least about 5×10¹⁴ vg/kg, at least about 6×10¹⁴ vg/kg, or at least about 7×10¹⁴ vg/kg. In certain embodiments, the AAV vector delivered at any of these doses is an AAV9 vector or an AAV rh74 vector.

In some embodiments, the AAV vector (e.g., AAV9, AAVrh.74, or AAVrh.10 vector) is administered intravenously at a dose of about 1×10¹² vg/kg, about 3×10¹² vg/kg, about 5×10¹² vg/kg, about 7×10¹² vg/kg, about 1×10¹³ vg/kg, about 3×10¹³ vg/kg, about 5×10¹³ vg/kg, about 7×10¹³ vg/kg, about 1×10¹⁴ vg/kg, about 3×10¹⁴ vg/kg, about 5×10¹⁴ vg/kg, about 7×10¹⁴ vg/kg, about 1×10¹³ vg/kg, about 3×10¹⁵ vg/kg, about 5×10¹⁵ vg/kg, or about 7×10¹⁵ vg/kg.

In some embodiments, the AAV vector (e.g., AAV9, AAVrh.74, or AAVrh.10 vector) is administered intravenously at a dose of 1×10¹² vg/kg, 3×10¹² vg/kg, 5×10¹² vg/kg, 7×10¹² vg/kg, 1×10¹³ vg/kg, 3×10¹³ vg/kg, 5×10¹³ vg/kg, 7×10¹³ vg/kg, 1×10¹⁴ vg/kg, 3×10¹⁴ vg/kg, 5×10¹⁴ vg/kg, 7×10¹⁴ vg/kg, 1×10¹⁵ vg/kg, 3×10¹⁵ vg/kg, 5×10¹⁵ vg/kg, or 7×10¹⁵ vg/kg. In certain embodiments, the AAV vector delivered at any of these doses is an AAV9 vector or an AAV rh74 vector.

Evidence of functional improvement, clinical benefit or efficacy in patients may be revealed by improvements in New York Heart Association functional classification (NYHA Class), echocardiography (stabilized or improved cardiac output, left ventricle ejection fraction, fractional shortening, left ventricular outflow tract obstruction, left ventricular wall thickness, left or right ventricular volumes, velocity time integral, time constant of ventricular relaxation, regurgitant volume, and decrease in left ventricular end diastolic diameter, LVEDD and decreased right ventricular area), electrocardiography (stabilized or improved ST-segment alterations, T-wave inversion, Q waves, atrial fibrillation, and/or supraventricular tachycardia), cardiac MRI, heart biopsy, decrease in paroxysmal ventricular arrhythmias, decrease in sudden cardiac death, and/or decrease in or lack of further development myocardial disarray.

Administration of an effective dose of the compositions may be by routes standard in the art including, but not limited to, systemic, local, direct injection, intravenous, intracardiac administration. In some cases, administration comprises systemic, local, direct injection, intravenous, intracardiac injection. Administration may be performed by cardiac catheterization.

In some embodiments, the disclosure provides for local administration and systemic administration of an effective dose of rAAV and compositions of the invention. For example, systemic administration may be administration into the circulatory system so that the entire body is affected. Systemic administration includes parental administration through injection, infusion or implantation. Routes of administration for the compositions disclosed herein include intravenous (“IV”) administration, intraperitoneal (“IP”) administration, intramuscular (“IM”) administration, intralesional administration, or subcutaneous (“SC”) administration, or the implantation of a slow-release device, e.g., a mini-osmotic pump, a depot formulation, etc. In some embodiments, the methods of the disclosure comprise administering an AAV vector of the disclosure, or pharmaceutical composition thereof by intravenous, intramuscular, intraarterial, intrarenal, intraurethral, intracardiac, intracoronary, intramyocardial, intradermal, epidural, subcutaneous, intraperitoneal, intraventricular, or ionophoretic administration.

In particular, administration of rAAV of the present invention may be accomplished by using any physical method that will transport the rAAV recombinant vector into the target tissue of an animal. Administration includes, but is not limited to, injection into the heart. In some embodiments, the methods include administering the rAAV into a blood vessel of the coronary circulation in vivo, such as retrograde coronary sinus infusion.

In some embodiments, the methods of the disclosure comprise intracardiac delivery. Infusion may be performed using specialized cannula, catheter, syringe/needle using an infusion pump. Administration may comprise delivery of an effective amount of the rAAV virion, or a pharmaceutical composition comprising the rAAV virion, to the heart. These may be achieved, e.g., via intravenous, intramuscular, intraarterial, intrarenal, intraurethral, intracardiac, intracoronary, intramyocardial, intradermal, epidural, subcutaneous, intraperitoneal, intraventricular, or ionophoretic administration. The compositions of the disclosure may further be administered intravenously.

Effects of rAAV Administration

In some embodiments, administration of rAAV of the present disclosure has beneficial effects for the subject.

### Lifespan

In some embodiments, administration of rAAV of the present disclosure may increase lifespan of the subject compared to a subject that is not administered the rAAV of the present disclosure or to baseline.

In some embodiments, administration of rAAV of the present disclosure increases lifespan by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, or at least about 500% compared to a subject that is not administered the rAAV of the present disclosure or to baseline.

In some embodiments, administration of rAAV of the present disclosure increases lifespan by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 200%, about 200% to about 300%, about 300% to about 400%, or by about 400% to about 500% compared to a subject that is not administered the rAAV of the present disclosure or to baseline.

### Ejection Fraction

In some embodiments, administration of rAAV of the present disclosure limits a decrease in, restores, and/or increases the ejection fraction in a subject compared to a subject that is not administered the rAAV of the present disclosure or to baseline. In some embodiments, administration of rAAV of the present disclosure limits a decrease in, restores, and/or increases the ejection fraction in a subject over time.

In some embodiments, administration of rAAV of the present disclosure restores and/or increases the ejection fraction by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the ejection fraction to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the ejection fraction by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the ejection fraction to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the ejection fraction by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the ejection fraction by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the ejection fraction to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% compared to a subject that is not administered the rAAV of the present disclosure or to baseline.

In some embodiments, administration of rAAV of the present disclosure limits a decrease in the ejection fraction to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% in a subject over time.

### Left Ventricular Ejection Fraction (LVEF)

In some embodiments, administration of rAAV of the present disclosure prevents a decrease in, restores, and/or increases in the LVEF in a subject compared to a subject that is not administered the rAAV of the present disclosure or to baseline. In some embodiments, administration of rAAV of the present disclosure limits a decrease in, restores, and/or increases in the LVEF in a subject over time.

In some embodiments, administration of rAAV of the present disclosure restores and/or increases the LVEF by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the LVEF to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the LVEF by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the LVEF to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the LVEF by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the LVEF by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the LVEF to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% compared to a subject that is not administered the rAAV of the present disclosure or to baseline.

In some embodiments, administration of rAAV of the present disclosure limits a decrease in the LVEF to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% in a subject over time.

### Right Ventricular Ejection Fraction (RVEF)

In some embodiments, administration of rAAV of the present disclosure prevents a decrease in, restores, and/or increases in the RVEF in a subject compared to a subject that is not administered the rAAV of the present disclosure or to baseline. In some embodiments, administration of rAAV of the present disclosure limits a decrease in, restores, and/or increases in the LVEF in a subject over time.

In some embodiments, administration of rAAV of the present disclosure restores and/or increases the RVEF by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the RVEF to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the RVEF by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the RVEF to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the RVEF by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the RVEF by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the RVEF to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% compared to a subject that is not administered the rAAV of the present disclosure or to baseline.

In some embodiments, administration of rAAV of the present disclosure limits a decrease in the RVEF to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% in a subject over time.

### Right Ventricle (RV) Area

In some embodiments, administration of rAAV of the present disclosure prevents an increase in, restores, and/or decreases the RV area in a subject compared to a subject that is not administered the rAAV of the present disclosure or to baseline. In some embodiments, administration of rAAV of the present disclosure prevents an increase in, restores, and/or decreases the RV area in a subject over time.

In some embodiments, administration of rAAV of the present disclosure prevents an increase in, restores, and/or decreases the RV area by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in, restores, and/or decreases the RV area by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in, restores, and/or decreases the RV area by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in, restores, and/or decreases the RV area by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Left Ventricle (LV) Area

In some embodiments, administration of rAAV of the present disclosure prevents an increase in, restores, and/or decreases the LV area in a subject compared to a subject that is not administered the rAAV of the present disclosure or to baseline. In some embodiments, administration of rAAV of the present disclosure prevents an increase in, restores, and/or decreases the RV area in a subject over time.

In some embodiments, administration of rAAV of the present disclosure prevents an increase in, restores, and/or decreases the LV area by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in, restores, and/or decreases the LV area by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in, restores, and/or decreases the LV area by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in, restores, and/or decreases the LV area by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Right Ventricle (RV) Velocity Time Integral (VTI)

In some embodiments, administration of rAAV of the present disclosure limits a decrease in, restores, and/or increases in the RV VTI in a subject compared to a subject that is not administered the rAAV of the present disclosure or to baseline. In some embodiments, administration of rAAV of the present disclosure limits a decrease in, restores, and/or increases in the RV VTI in a subject over time.

In some embodiments, administration of rAAV of the present disclosure restores and/or increases the RV VTI by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the RV VTI to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the RV VTI by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the RV VTI to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the RV VTI by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the RV VTI by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the RV VTI to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% compared to a subject that is not administered the rAAV of the present disclosure or to baseline.

In some embodiments, administration of rAAV of the present disclosure limits a decrease in the RV VTI to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% in a subject over time.

### Left Ventricle (LV) Velocity Time Integral (VTI)

In some embodiments, administration of rAAV of the present disclosure limits a decrease in, restores, and/or increases in the LV VTI in a subject compared to a subject that is not administered the rAAV of the present disclosure or to baseline. In some embodiments, administration of rAAV of the present disclosure limits a decrease in, restores, and/or increases in the LV VTI in a subject over time.

In some embodiments, administration of rAAV of the present disclosure restores and/or increases the LV VTI by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure limits a decrease in the LV VTI to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% compared to a subject that is not administered the rAAV of the present disclosure or to baseline.

In some embodiments, administration of rAAV of the present disclosure restores and/or increases the LV VTI by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure limits a decrease in the LV VTI to less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, at least about 75%, less than about 80%, less than about 85%, less than about 90%, less than about 95%, or less than about 100% in a subject over time.

In some embodiments, administration of rAAV of the present disclosure restores and/or increases the LV VTI by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure restores and/or increases the LV VTI by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

In some embodiments, administration of rAAV of the present disclosure limits a decrease in the LV VTI to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% compared to a subject that is not administered the rAAV of the present disclosure or to baseline.

In some embodiments, administration of rAAV of the present disclosure limits a decrease in the LV VTI to less than about 1% to less than about 90%, less than about 20% to less than about 80%, less than about 30% to less than about 80%, less than about 40% to less than about 80%, less than about 50% to less than about 80%, less than about 1% to less than about 2%, less than about 2% to less than about 3%, less than about 3% to less than about 4%, less than about 4% to less than about 5%, less than about 5% to less than about 6%, less than about 6% to less than about 7%, less than about 7% to less than about 8%, less than about 8% to less than about 9%, less than about 9% to 10%, less than about 10% to less than about 15%, less than about 15% to less than about 20%, less than about 20% to less than about 35%, less than about 25% to less than about 30%, less than about 30% to less than about 35%, less than about 35% to less than about 40%, less than about 40% to less than about 45%, less than about 45% to less than about 50%, less than about 50% to less than about 55%, less than about 55% to less than about 60%, less than about 60% to less than about 65%, less than about 65% to less than about 70%, less than about 70% to less than about 75%, less than about 75% to less than about 80%, less than about 80% to less than about 85%, less than about 85% to less than about 90%, less than about 90% to less than about 95%, or by less than about 95% to about 100% in a subject over time.

### Left Ventricle (LV) Fibrosis

In some embodiments, administration of rAAV of the present disclosure prevents an increase in and/or decreases the LV fibrosis in a subject compared to a subject that is not administered the rAAV of the present disclosure or to baseline. In some embodiments, administration of rAAV of the present disclosure prevents an increase in and/or decreases the LV fibrosis in a subject over time.

In some embodiments, administration of rAAV of the present disclosure prevents an increase in and/or decreases the LV fibrosis by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases the LV fibrosis by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in and/or decreases the LV fibrosis by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases the LV fibrosis by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Right Ventricle (RV) Fibrosis

In some embodiments, administration of rAAV of the present disclosure prevents an increase in and/or decreases the RV fibrosis in a subject compared to a subject that is not administered the rAAV of the present disclosure or to baseline. In some embodiments, administration of rAAV of the present disclosure prevents an increase in and/or decreases the RV fibrosis in a subject over time.

In some embodiments, administration of rAAV of the present disclosure prevents an increase in and/or decreases the RV fibrosis by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases the RV fibrosis by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in and/or decreases the RV fibrosis by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases the RV fibrosis by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Premature Ventricular Contractions (PVC)

In some embodiments, administration of rAAV of the present disclosure prevents an increase in and/or decreases PVC in a subject compared to a subject that is not administered the rAAV of the present disclosure or to baseline. In some embodiments, administration of rAAV of the present disclosure prevents an increase in and/or decreases PVC in a subject over time.

In some embodiments, administration of rAAV of the present disclosure prevents an increase in and/or decreases PVC by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases PVC by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in and/or decreases PVC by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases PVC by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Non-Sustained Ventricular Tachycardia (NSVT)

In some embodiments, administration of rAAV of the present disclosure prevents an increase in and/or decreases NSVT in a subject compared to a subject that is not administered the rAAV of the present disclosure or to baseline. In some embodiments, administration of rAAV of the present disclosure prevents an increase in and/or decreases NSVT in a subject over time.

In some embodiments, administration of rAAV of the present disclosure prevents an increase in and/or decreases NSVT by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases NSVT by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in and/or decreases NSVT by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases NSVT by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### Ectopic Beats

In some embodiments, administration of rAAV of the present disclosure prevents an increase in and/or decreases ectopic beats in a subject compared to a subject that is not administered the rAAV of the present disclosure or to baseline. In some embodiments, administration of rAAV of the present disclosure prevents an increase in and/or decreases ectopic beats in a subject over time.

In some embodiments, administration of rAAV of the present disclosure prevents an increase in and/or decreases ectopic beats by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to a subject that is not administered the rAAV of the present disclosure or to baseline.

In some embodiments, administration of rAAV of the present disclosure prevents an increase in and/or decreases ectopic beats by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% compared to a subject that is not administered the rAAV of the present disclosure or to baseline.

In some embodiments, administration of rAAV of the present disclosure prevents an increase in and/or decreases ectopic beats by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in a subject over time.

In some embodiments, administration of rAAV of the present disclosure prevents an increase in and/or decreases ectopic beats by about 1% to about 90%, about 20% to about 80%, about 30% to about 80%, about 40% to about 80%, about 50% to about 80%, about 1% to about 2%, about 2% to about 3%, about 3% to about 4%, about 4% to about 5%, about 5% to about 6%, about 6% to about 7%, about 7% to about 8%, about 8% to about 9%, about 9% to 10%, about 10% to about 15%, about 15% to about 20%, about 20% to about 35%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or by about 95% to about 100% in a subject over time.

### NUMBERED EMBODIMENTS

Embodiment 1. A polynucleotide, comprising an expression cassette and optionally flanking adeno-associated virus (AAV) inverted terminal repeats (ITRs), wherein the polynucleotide comprises a polynucleotide sequence encoding a B-cell Lymphoma 2-Associated Anthanogene 3 (BAG3), or a functional variant thereof, operatively linked to a promoter, optionally wherein the promoter is a heterologous promoter.

Embodiment 2. The polynucleotide of embodiment 1, wherein the promoter is a cardiac-specific promoter.

Embodiment 3. The polynucleotide of embodiment 1 or embodiment 2, wherein the promoter is a muscle-specific promoter.

Embodiment 4. The polynucleotide of any one of embodiments 1 to 3, wherein the promoter is a cardiomyocyte-specific promoter.

Embodiment 5. The polynucleotide of any one of embodiments 1 to 4, wherein the promoter is a Myosin Heavy-chain Creatine Kinase 7 (MHCK7) promoter.

Embodiment 6. The polynucleotide of embodiment 5, wherein the MHCK7 promoter shares at least 75%, 80%, 85%, 90%, 95%, 96%, 97%, 98%, 99% or 100% identity with SEQ ID NO: 31.

Embodiment 7. The polynucleotide of any one of embodiments 1 to 4, wherein the promoter is a cardiac troponin T (hTNNT2) promoter.

Embodiment 8. The polynucleotide of embodiment 7, wherein the hTNNT2 promoter shares at least 75%, 80%, 85%, 90%, 95%, 96%, 97%, 98%, 99% or 100% identity with SEQ ID NO: 32.

Embodiment 9. The polynucleotide of any one of embodiments 1 to 4, wherein the promoter is a human heat shock factor 70 (hHSP70) promoter.

Embodiment 10. The polynucleotide of embodiment 9, wherein the hHSP70 promoter shares at least 75%, 80%, 85%, 90%, 95%, 96%, 97%, 98%, 99% or 100% identity with SEQ ID NO: 115.

Embodiment 11. The polynucleotide of any one of embodiments 1 to 10, wherein the expression cassette comprises exon 1 of the cardiac troponin T (hTNNT2) gene, wherein optionally the hTNNT2 promoter and exon 1 together share at least 75%, 80%, 85%, 90%, 95%, 96%, 97%, 98%, 99% or 100% identity with SEQ ID NO: 32.

Embodiment 12. The polynucleotide of any one of embodiments 1 to 4, wherein the promoter is a ubiquitous promoter, optionally a CMV promoter or a CAG promoter or a chicken β-globin promoter or a UBC promoter (SEQ ID NO: 116).

Embodiment 13. The polynucleotide of any one of embodiments 1 to 12, wherein the expression cassette comprises a polyA signal.

Embodiment 14. The polynucleotide of embodiment 13, wherein the polyA signal is a human growth hormone (hGH) polyA.

Embodiment 15. The polynucleotide of any one of embodiments 1 to 14, wherein the expression cassette comprises a Woodchuck Hepatitis Virus Posttranscriptional Regulatory Element (WPRE), optionally a mutant or modified WPRE (WPRE(x)), optionally WPRE Mut6 (SEQ ID NO:63).

Embodiment 16. The polynucleotide of any one of embodiments 1 to 15, wherein the expression cassette comprises a polynucleotide sequence encoding a Green Fluorescence Protein (GFP).

Embodiment 17. The polynucleotide of any one of embodiments 1 to 16, wherein the B-cell Lymphoma 2-Associated Anthanogene 3 (BAG3) or functional fragment or variant thereof is a full length or wild-type BAG3.

Embodiment 18. The polynucleotide of any one of embodiments 1-17, wherein the BAG3 is a human BAG3.

Embodiment 19. The polynucleotide of any one of embodiments 1 to 18, wherein the polynucleotide sequence encoding BAG3 shares at least 75%, 80%, 85%, 90%, 95%, 96%, 97%, 98%, 99% or 100% identity with SEQ ID NO: 2.

Embodiment 20. The polynucleotide of any one of embodiments 1 to 19, wherein the polynucleotide sequence encoding BAG3 is a human BAG3 polynucleotide.

Embodiment 21. The polynucleotide of any one of embodiments 1 to 20, wherein the polynucleotide comprises at least about 3.0 kb, at least about 3.2 kb, at least about 3.4 kb, at least about 3.5 kb, at least about 3.7 kb, at least about 4.0 kb, at least about 4.1 kb, at least about 4.2 kb, at least about 4.3 kb, at least about 4.4 kb, at least about 4.5 kb, at least about 4.6 kb, at least about 4.7 kb, at least about 4.8 kb, or at least about 5.0 kb.

Embodiment 22. The polynucleotide of any one of embodiments 1 to 21, wherein the polynucleotide comprises at most about 3.1 kb, at most about 3.3 kb, at most about 3.5 kb, at most about 3.7 kb, at most about 3.9 kb, at most about 4.1 kb, at most about 4.2 kb, at most about 4.3 kb, at most about 4.4 kb, at most about 4.5 kb, at most about 4.6 kb, at most about 4.7 kb, at most about 4.8 kb, at most about 4.9 kb, or at most about 5.0 kb.

Embodiment 23. The polynucleotide of any one of embodiments 1 to 22, wherein the polynucleotide comprises 4.4 kb to 5.0 kb, 4.4 kb to 4.9 kb, or 4.4 kb to 4.8 kb, wherein the polynucleotide comprises 4.0 kb to 4.6 kb, 4.0 kb to 4.5 kb, or 4.0 kb to 4.4 kb, wherein the polynucleotide comprises 4.0 kb to 4.3 kb, 4.0 kb to 4.2 kb, or 4.0 kb to 4.1 kb, or wherein the polynucleotide comprises 3.0 kb to 3.9 kb, 3.0 kb to 3.8 kb, or 3.0 kb to 3.7 kb.

Embodiment 24. The polynucleotide of any one of embodiments 1 to 23, wherein the expression cassette is flanked by 5′ and 3′ inverted terminal repeats (ITRs).

Embodiment 25. The polynucleotide of embodiment 24, wherein the ITRs are AAV2 ITRs and/or the ITRs share at least 75%, 80%, 85%, 90%, 95%, 96%, 97%, 98%, 99% or 100% identity with any one of SEQ ID NO: 15-21.

Embodiment 26. A gene therapy vector, comprising the polynucleotide of any one of embodiments 1 to 25.

Embodiment 27. The vector of embodiment 26, wherein the gene therapy vector is a recombinant adeno-associated virus (rAAV) vector.

Embodiment 28. The vector of embodiment 27, wherein the rAAV vector is an AAVrh74 or a functional variant thereof.

Embodiment 29. The vector of embodiment 28, wherein the rAAV vector comprises a capsid protein that shares 90%, 91%, 92%, 93%, 94%, 95%, 96%, 97%, 98%, 99% or 100% identity to SEQ ID NO: 100.

Embodiment 30. The vector of embodiment 27, wherein the rAAV vector is an AAV9 or a functional variant thereof.

Embodiment 31. The vector of embodiment 30, wherein the rAAV vector comprises a capsid protein that shares 90%, 91%, 92%, 93%, 94%, 95%, 96%, 97%, 98%, 99% or 100% identity to any one of SEQ ID NO: 97.

Embodiment 32. The vector of embodiment 27, wherein the rAAV vector is an AAV6 or a functional variant thereof.

Embodiment 33. The vector of embodiment 32, wherein the rAAV vector comprises a capsid protein that shares 90%, 91%, 92%, 93%, 94%, 95%, 96%, 97%, 98%, 99% or 100% identity to SEQ ID NO: 98.

Embodiment 34. The vector of embodiment 27, wherein the rAAV vector is an AAVrh10 or a functional variant thereof.

Embodiment 35. The vector of embodiment 34, wherein the rAAV vector comprises a capsid protein that shares 90%, 91%, 92%, 93%, 94%, 95%, 96%, 97%, 98%, 99% or 100% identity to SEQ ID NO: 99.

Embodiment 36. A method of treating and/or preventing a disease or disorder in a subject in need thereof, comprising administering the vector of any one of embodiments 26-35 to the subject.

Embodiment 37. The method of embodiment 36, wherein the disease or disorder is a cardiac disorder.

Embodiment 38. The method of embodiment 37, wherein the cardiac disorder is a cardiomyopathy, optionally BAG3-related dilated cardiomyopathy.

Embodiment 39. The method of embodiment 38, wherein the cardiomyopathy is a hypertrophic cardiomyopathy (HCM) (hypertrophic).

Embodiment 40. The method of embodiment 38, wherein the cardiomyopathy is a dilated cardiomyopathy (DCM).

Embodiment 41. The method of embodiment 37, wherein the disease or disorder is arrhythmia, optionally atrial fibrillation or sinus node disease.

Embodiment 42. The method of embodiment 37, wherein the disease or disorder is heart failure.

Embodiment 43. The method of any one of embodiments 36 to 42, wherein the subject is a mammal.

Embodiment 44. The method of embodiment 43, wherein the subject is a primate.

Embodiment 45. The method of embodiment 44, wherein the subject is a human.

Embodiment 46. The method of any one of embodiments 36 to 45, wherein the subject has a mutation in a BAG3 gene and/or reduced expression of BAG3 as compared to a healthy subject.

Embodiment 47. The method of any one of embodiment 36 to 46, wherein the vector is administered by intravenous injection, intracardiac injection, intracardiac infusion, and/or cardiac catheterization.

Embodiment 48. The method of any one of embodiments 36 to 47, wherein the administration increases BAG3 expression by at least about 5%.

Embodiment 49. The method of any one of embodiments 36 to 47, wherein the administration increases BAG3 expression by at least about 30%.

Embodiment 50. The method of any one of embodiments 36 to 47, wherein the administration increases BAG3 expression by at least about 70%.

Embodiment 51. The method of any one of embodiments 36 to 47, wherein the administration increases BAG3 expression by about 5% to about 10%.

Embodiment 52. The method of any one of embodiments 36 to 47, wherein the administration increases BAG3 expression by about 30% to about 50%.

Embodiment 53. The method of any one of embodiments 36 to 47, wherein the administration increases BAG3 expression by about 50% to about 70%.

Embodiment 54. The method of any one of embodiments 36 to 47, wherein the administration increases BAG3 expression by about 70% to about 100%.

Embodiment 55. The method of any one of embodiments 36 to 54, wherein the method treats and/or prevents the disease or disorder.

Embodiment 56. The method of any one of embodiments 36 to 55, wherein the method comprises administering an effective amount of the vector.

Embodiment 57. The method of any one of embodiments 36 to 56, wherein the disease or disorder is related to or caused by truncation of BAG3 in the subject.

Embodiment 58. The method of any one of embodiments 36 to 57, wherein the method comprises administering a pharmaceutical composition comprising an effective amount of the vector.

Embodiment 59. The method of any one of embodiments 36 to 58, wherein the method comprises administering between about 1×10¹¹ vector genomes and about 1×10¹³ vector genomes of the vector to the subject, administering between about 1×10¹² vector genomes and about 1×10¹⁴ vector genomes of the vector to the subject, or administering between about 1×10¹³ vector genomes and about 1×10¹⁵ vector genomes of the vector to the subject.

Embodiment 60. A pharmaceutical composition comprising the vector of any one of embodiments 26 to 35.

Embodiment 61. A kit comprising the vector of any one of embodiments 26 to 35 or the pharmaceutical composition of embodiment 60 and optionally instructions for use.

Embodiment 62. Use of the vector of any one of embodiments 26 to 35 in treating a disease or disorder, optionally according to the method of any one of embodiments 39 to 63.

Embodiment 63. A vector according to any one of embodiments 29 to 38 for use in treating a disease or disorder, optionally according to the method of any one of embodiments 36 to 59.

Embodiment 64. A polynucleotide, comprising a polynucleotide sequences that shares at least 90%, 91%, 92%, 93%, 94%, 95%, 96%, 97%, 98%, 99%, or 100% identity to any one of SEQ ID NOs: 107-116.

Embodiment 65. The polynucleotide of embodiment 64, wherein the promoter is a MHCK7 promoter.

Embodiment 66. The polynucleotide of embodiment 65, wherein the MHCK7 promoter shares at least 75%, 80%, 85%, 90%, 95%, 96%, 97%, 98%, 99% or 100% identity with SEQ ID NO: 31.

Embodiment 67. The polynucleotide of embodiment 64, wherein the BAG3 is a human BAG3.

Embodiment 68. A gene therapy vector, comprising the polynucleotide of any one of embodiments 64 to 67.

Embodiment 69. The vector of embodiment 68, wherein the gene therapy vector is a recombinant adeno-associated virus (rAAV) vector.

Embodiment 70. The vector of embodiment 69, wherein the rAAV vector is an AArh74 vector.

Embodiment 71. The vector of embodiment 69, wherein the rAAV vector is an AAV9 vector.

Embodiment 72. A method of treating and/or preventing a cardiac disorder in a subject identified as having a mutation or truncation in BAG3, comprising administering the vector of any one of embodiments 68 to 71 to the subject.

Embodiment 73. The method of embodiment 72, wherein the disease or disorder is a cardiomyopathy, optionally a dilated cardiomyopathy (DCM).

Embodiment 74. The method of embodiment 72, wherein the cardiac disorder is arrhythmia, optionally atrial fibrillation or sinus node disease, or BAG3-related dilated cardiomyopathy.

Embodiment 75. The method of embodiment 72, wherein the cardiac disorder is heart failure.

Embodiment 76. The method of any one of embodiments 72 to 75, wherein the subject is a mammal.

Embodiment 77. The method of any one of embodiments 72 to 76, wherein the vector is administered by intravenous injection, intracardiac injection, intracardiac infusion, and/or cardiac catheterization.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the present disclosure have been described herein for purposes of illustration, various modifications may be made without deviating from the spirit and scope of the present disclosure.

### EXAMPLES

### Example 1: Pre-Clinical Transgene Expression

Vectors illustrated in FIGS. 1-8 are tested. AAV vectors or respective expression cassettes are tested in vitro using cultured cardiomyocytes (e.g., induced pluripotent stem cell cardiomyocytes, iPSC-CMs, AC16, HL-1, C2C12, H9C2) or other cells amenable to transfection or transduction with these constructs. Expression of BAG3 transgene protein is assessed by ELISA, immunofluorescence, immunohistochemistry, and Western blot. Vector DNA is detected by PCR and BAG3 transgene mRNA is detected by qRT-PCR.

Cell-based studies employing patient iPSC-derived cardiomyocytes can be very challenging given the difficulty of effectively maintaining the BAG3 KO cardiomyocytes in culture, presumably due to high levels of spontaneous apoptosis. To reveal benefit of expression of BAG3 transgene (either following AAV vector transduction and/or transfection with vector plasmids) cell models using C2C12, AC16 and H9C2 cells and an shRNA targeting BAG3 are used with the goal to mitigate/reduce the apoptosis levels (measured by conventional methods, e.g., TUNEL) of these cells after cardiomyocyte differentiation.

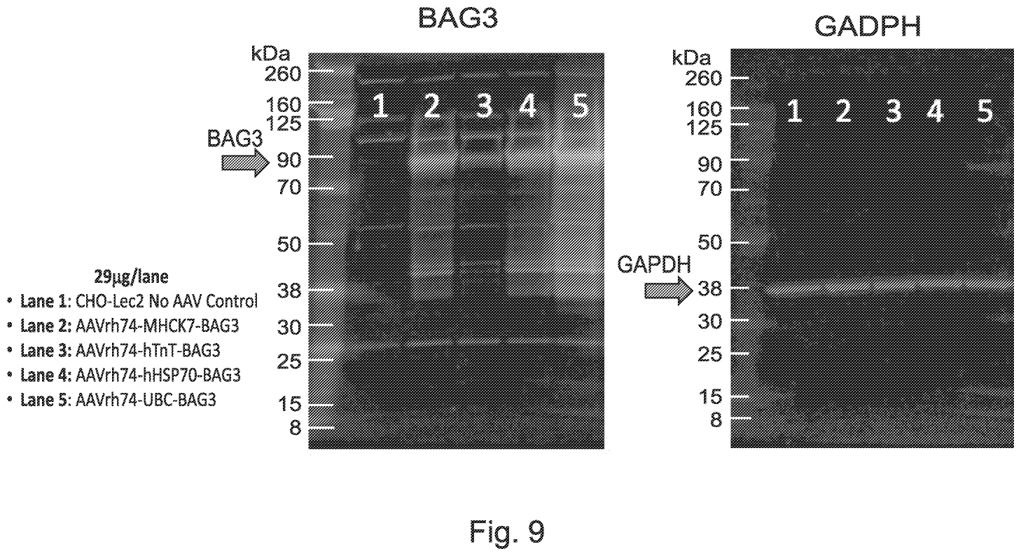

Expression cassettes illustrated in FIG. 1 to 3 and FIG. 8 were tested following packaging into an AAV.rh74 vector. The resulting AAV vectors were tested in vitro using cultured CHO-Lec2 (standard mutant cells that have a 70-90% deficiency of sialic acid in their glycoproteins and gangliosides that make this cell more susceptible to AAV transduction). Subsequent expression of BAG3 protein in transduced CHO-Lec2 cells was assessed by Western blot. Data illustrating AAVrh.74-BAG3 mediated expression of the transgene protein (BAG3) are presented (FIG. 9).

The UBC promoter produced the highest expression levels of BAG3 by WB in the CHO-Lec2 cells, followed by the hHSP70 and MHCK7. The hTnnT2 promoter (“hTnT”) was found to drive lower levels of BAG3 protein expression in these conditions. Based on these results, it can be concluded that AAVrh.74 vectors can effectively express BAG3 protein.

### Example 2: Pre-Clinical Efficacy In Vivo in the Bag3 KO and Bag3 CKO Mouse

Selected vectors are tested in vivo using mutant mouse models of cardiomyopathy. A BAG3−/− knock-out mouse model exhibits severe DCM phenotype and presents one or more DCM elements of human disease. The BAG3−/− knock-out mouse (described in, e.g., Homma et al., Am J Pathol, 2006) has s single retrovirus insertion that selectively disrupted the mouse bag3 gene. Histological analysis of the tissues of BAG3−/− mice revealed abnormalities in skeletal and cardiac tissue but not elsewhere, suggesting the possibility of myopathy. The BAG3−/− knock-out mice were indistinguishable from their wild-type (+/+) and heterozygous (+/−) littermates during the 1st week of life. The cardiac muscle from BAG3-null mice showed degenerative changes, with the atrium affected to a greater degree than the ventricle. The KO mice ceased to gain weight after day 12 and appeared dwarfed relative to littermates and all BAG3−/− animals are dead by day 25. After 20 days of age, myofiber degeneration was found throughout all muscles sampled in BAG3−/− mice. H&E-stained fresh-frozen sections of the muscle of BAG3−/− animals showed a marked variation in myofiber size, with evidence of atrophic fibers and intracellular accumulations of basophilic material. Inflammation, myonecrosis or other dystrophic pathological abnormalities were not observed in BAG3−/− muscle.

A Cre inducible heart-specific BAG3−/− knock-out (also termed Bis-iCKO) is also tested as a mouse model of moderately severe DCM phenotype. This mouse model exhibits one or more DCM elements of human disease and is characterized by a cardiomyocyte-specific, tamoxifen-activated, BAG3 knockout and is referred to as “Bis-iCKO” (described in, e.g., Yun et al, Int J Mol Sci 2021). This mouse model allows control of the onset of BAG3 loss of expression, limits loss of BAG3 to adult myocytes, and initiates a progression of molecular and functional events leading to dilated cardiomyopathy. Bis-iCKO mice were viable at birth; however, they had a striking susceptibility to premature death consequent to DCM and heart failure. While sixty percent of the Bis-iCKO mice survived until 28 days of life, morphological and histological analysis of Bis-iCKO hearts revealed marked cardiac enlargement in these mice. Echocardiography revealed an age-dependent decrease in left ventricular (LV) systolic function (percentage of fractional shortening [FS]) in mice deficient for BAG3.

Benefit of AAV-mediated BAG3 expression in either the above or similar models would be evidenced by an increase in survival, an increase in body weight gain, mitigation of the normal progression of cardiomyopathy observed on echocardiograms (e.g., LVESD, LVEDD), mitigation of enlarged size of right and/or left ventricle and/or mitigation of typical decrease in left ventricular ejection fraction and/or fractional shortening. Histological analyses would reveal benefit by diminished appearance of disease-related myofiber disarray, attenuated ventricular dilation, reduction in thinning of ventricular wall, reduction in number of apoptotic cells, reduction of DNA damage and reduction in disease-related change in absolute size of heart.

### Example 3: Rescue of Heart Failure In Vivo after Transverse Aortic Constriction (Tac)

AAV-BAG3 gene therapy with select AAV vectors described above is performed essentially as described in Knezevic et al. (JACC, 2016). AAV expression cassettes are packaged and delivered in vivo using different capsid serotypes such as AAVrh.74 and/or AAV9.

Mouse TAC Model: Transaortic constriction (TAC) in the mouse is an experimentally induced cardiac hypertrophy due to pressure overload with subsequent heart failure. Compared to other experimental mouse models of heart failure, the TAC model results in more reproducible cardiac hypertrophy and a gradual time course of development of heart failure. Following TAC in the mouse, a progressive decrease in ejection fraction and other measures of heart function are paralleled by a progressive decrease of cardiac BAG3 levels. Male C57BI/6J mice (approximately 4 months of age) are anesthetized and the aortic arch is visualized by performing an anterior thoracotomy to the level of the third intercostal space. Constriction is performed by tying a silk suture against a 28-gauge needle between the first and second trunk of the aortic arch. For consistency, constriction levels are quantified by measuring alterations in Doppler velocities of the right and left carotid arteries 7 days post-surgery. Right-to-left carotid peak velocity ratios may range from 5.0 to 6.5 and 2-week post TAC ejection fractions may range from 40%-50%.

Functional Evidence of Efficacy by Echocardiography: Evidence of bioactivity and efficacy for cardiac benefit in the TAC model is evaluated using transthoracic echocardiography at predefined timepoints including baseline and various intervals after TAC. To screen animals with sufficient heart failure suitable for this mouse model, Doppler ratios of right carotid to left carotid peak velocity (RC/LC) are determined 1-week post-TAC and those that do not meet criteria (RC/LC of 5.0-6.5) are excluded from study. Additionally, echocardiography at 2 weeks post-TAC is performed and animals outside the range of 40-50% ejection fraction (EF) are also excluded. Mice with appropriate Doppler RC/EV and EF by echocardiogram are then injected (either intra-venously or intra-retro-orbitally) at week 8 post-TAC with AAV constructs overexpressing BAG3 protein or with formulation buffer (FB; vehicle control). Efficacy will be evident in AAV-BAG3 treated animals by significantly increased EF and FS compared to the FB control group across time. Echocardiography will reveal that FB injected mice will be found to have an EF and FS that declines progressively across time. In contrast, AAV-BAG3 injected animals will be found to have an EF and FS returning to the control levels during the weeks following treatment. Histological analyses will reveal attenuation of myocardial fiber disarray.

1. A polynucleotide, comprising an expression cassette and optionally flanking adeno-associated virus (AAV) inverted terminal repeats (ITRs), wherein the polynucleotide comprises a polynucleotide sequence encoding a B-cell Lymphoma 2-Associated Anthanogene 3 (BAG3), or a functional variant thereof, operatively linked to a promoter, optionally wherein the promoter is a heterologous promoter.

2. The polynucleotide of claim 1, wherein the promoter is a cardiac-specific promoter.

3. The polynucleotide of claim 1 or claim 2, wherein the promoter is a muscle-specific promoter.

4. The polynucleotide of any one of claims 1 to 3, wherein the promoter is a cardiomyocyte-specific promoter.

5. The polynucleotide of any one of claims 1 to 4, wherein the promoter is a Myosin Heavy-chain Creatine Kinase 7 (MHCK7) promoter.

6. The polynucleotide of claim 5, wherein the MHCK7 promoter shares at least 75%, 80%, 85%, 90%, 95%, 96%, 97%, 98%, 99% or 100% identity with SEQ ID NO: 31.

7. The polynucleotide of any one of claims 1 to 4, wherein the promoter is a cardiac troponin T (hTNNT2) promoter.

8. The polynucleotide of claim 7, wherein the hTNNT2 promoter shares at least 75%, 80%, 85%, 90%, 95%, 96%, 97%, 98%, 99% or 100% identity with SEQ ID NO: 32.

9. The polynucleotide of any one of claims 1 to 4, wherein the promoter is a human heat shock factor 70 (hHSP70) promoter.

10. The polynucleotide of claim 9, wherein the hHSP70 promoter shares at least 75%, 80%, 85%, 90%, 95%, 96%, 97%, 98%, 99% or 100% identity with SEQ ID NO: 115.

11. The polynucleotide of any one of claims 1 to 10, wherein the expression cassette comprises exon 1 of the cardiac troponin T (hTNNT2) gene, wherein optionally the hTNNT2 promoter and exon 1 together share at least 75%, 80%, 85%, 90%, 95%, 96%, 97%, 98%, 99% or 100% identity with SEQ ID NO: 32.

12. The polynucleotide of any one of claims 1 to 4, wherein the promoter is a ubiquitous promoter, optionally a CMV promoter or a CAG promoter or a chicken b-globin promoter or a UBC promoter (SEQ ID NO: 116).

13. The polynucleotide of any one of claims 1 to 12, wherein the expression cassette comprises a polyA signal.

14. The polynucleotide of claim 13, wherein the polyA signal is a human growth hormone (hGH) polyA.

15. The polynucleotide of any one of claims 1 to 14, wherein the expression cassette comprises a Woodchuck Hepatitis Virus Posttranscriptional Regulatory Element (WPRE), optionally a mutant or modified WPRE (WPRE(x)), optionally WPRE Mut6 (SEQ ID NO:63).

16. The polynucleotide of any one of claims 1 to 15, wherein the expression cassette comprises a polynucleotide sequence encoding a Green Fluorescence Protein (GFP).

17. The polynucleotide of any one of claims 1 to 16, wherein the B-cell Lymphoma 2-Associated Anthanogene 3 (BAG3) or functional fragment or variant thereof is a full length or wild-type BAG3.

18. The polynucleotide of any one of claims 1 to 17, wherein the BAG3 is a human BAG3.

19. The polynucleotide of any one of claims 1 to 18, wherein the polynucleotide sequence encoding BAG3 shares at least 75%, 80%, 85%, 90%, 95%, 96%, 97%, 98%, 99% or 100% identity with SEQ ID NO: 2.

20. The polynucleotide of any one of claims 1 to 19, wherein the polynucleotide sequence encoding BAG3 is a human BAG3 polynucleotide.

21. The polynucleotide of any one of claims 1 to 20, wherein the polynucleotide comprises at least about 3.0 kb, at least about 3.2 kb, at least about 3.4 kb, at least about 3.5 kb, at least about 3.7 kb, at least about 4.0 kb, at least about 4.1 kb, at least about 4.2 kb, at least about 4.3 kb, at least about 4.4 kb, at least about 4.5 kb, at least about 4.6 kb, at least about 4.7 kb, at least about 4.8 kb, or at least about 5.0 kb.

22. The polynucleotide of any one of claims 1 to 21, wherein the polynucleotide comprises at most about 3.1 kb, at most about 3.3 kb, at most about 3.5 kb, at most about 3.7 kb, at most about 3.9 kb, at most about 4.1 kb, at most about 4.2 kb, at most about 4.3 kb, at most about 4.4 kb, at most about 4.5 kb, at most about 4.6 kb, at most about 4.7 kb, at most about 4.8 kb, at most about 4.9 kb, or at most about 5.0 kb.

23. The polynucleotide of any one of claims 1 to 22, wherein the polynucleotide comprises 4.4 kb to 5.0 kb, 4.4 kb to 4.9 kb, or 4.4 kb to 4.8 kb, wherein the polynucleotide comprises 4.0 kb to 4.6 kb, 4.0 kb to 4.5 kb, or 4.0 kb to 4.4 kb, wherein the polynucleotide comprises 4.0 kb to 4.3 kb, 4.0 kb to 4.2 kb, or 4.0 kb to 4.1 kb, or wherein the polynucleotide comprises 3.0 kb to 3.9 kb, 3.0 kb to 3.8 kb, or 3.0 kb to 3.7 kb.

24. The polynucleotide of any one of claims 1 to 23, wherein the expression cassette is flanked by 5′ and 3′ inverted terminal repeats (ITRs).

25. The polynucleotide of claim 24, wherein the ITRs are AAV2 ITRs and/or the ITRs share at least 75%, 80%, 85%, 90%, 95%, 96%, 97%, 98%, 99% or 100% identity with any one of SEQ ID NO: 15-21.

26. A gene therapy vector, comprising the polynucleotide of any one of claims 1 to 25.

27. The vector of claim 26, wherein the gene therapy vector is a recombinant adeno-associated virus (rAAV) vector.

28. The vector of claim 27, wherein the rAAV vector is an AAVrh74 or a functional variant thereof.

29. The vector of claim 28, wherein the rAAV vector comprises a capsid protein that shares 90%, 91%, 92%, 93%, 94%, 95%, 96%, 97%, 98%, 99% or 100% identity to SEQ ID NO: 100.

30. The vector of claim 27, wherein the rAAV vector is an AAV9 or a functional variant thereof.

31. The vector of claim 30, wherein the rAAV vector comprises a capsid protein that shares 90%, 91%, 92%, 93%, 94%, 95%, 96%, 97%, 98%, 99% or 100% identity to any one of SEQ ID NO: 97.

32. The vector of claim 27, wherein the rAAV vector is an AAV6 or a functional variant thereof.

33. The vector of claim 32, wherein the rAAV vector comprises a capsid protein that shares 90%, 91%, 92%, 93%, 94%, 95%, 96%, 97%, 98%, 99% or 100% identity to SEQ ID NO: 98.

34. The vector of claim 27, wherein the rAAV vector is an AAVrh10 or a functional variant thereof.

35. The vector of claim 34, wherein the rAAV vector comprises a capsid protein that shares 90%, 91%, 92%, 93%, 94%, 95%, 96%, 97%, 98%, 99% or 100% identity to SEQ ID NO: 99.

36. A method of treating and/or preventing a disease or disorder in a subject in need thereof, comprising administering the vector of any one of claims 26-35 to the subject.

37. The method of claim 36, wherein the disease or disorder is a cardiac disorder.

38. The method of claim 37, wherein the cardiac disorder is a cardiomyopathy, optionally BAG3-related dilated cardiomyopathy.

39. The method of claim 38, wherein the cardiomyopathy is a hypertrophic cardiomyopathy (HCM) (hypertrophic).

40. The method of claim 38, wherein the cardiomyopathy is a dilated cardiomyopathy (DCM).

41. The method of claim 37, wherein the disease or disorder is arrhythmia, optionally atrial fibrillation or sinus node disease.

42. The method of claim 37, wherein the disease or disorder is heart failure.

43. The method of any one of claims 36 to 42, wherein the subject is a mammal.

44. The method of claim 43, wherein the subject is a primate.

45. The method of claim 44, wherein the subject is a human.

46. The method of any one of claims 36 to 45, wherein the subject has a mutation in a BAG3 gene and/or reduced expression of BAG3 as compared to a healthy subject.

47. The method of any one of claim 36 to 46, wherein the vector is administered by intravenous injection, intracardiac injection, intracardiac infusion, and/or cardiac catheterization.

48. The method of any one of claims 36 to 47, wherein the administration increases BAG3 expression by at least about 5%.

49. The method of any one of claims 36 to 47, wherein the administration increases BAG3 expression by at least about 30%.

50. The method of any one of claims 36 to 47, wherein the administration increases BAG3 expression by at least about 70%.

51. The method of any one of claims 36 to 47, wherein the administration increases BAG3 expression by about 5% to about 10%.

52. The method of any one of claims 36 to 47, wherein the administration increases BAG3 expression by about 30% to about 50%.

53. The method of any one of claims 36 to 47, wherein the administration increases BAG3 expression by about 50% to about 70%.

54. The method of any one of claims 36 to 47, wherein the administration increases BAG3 expression by about 70% to about 100%.

55. The method of any one of claims 36 to 54, wherein the method treats and/or prevents the disease or disorder.

56. The method of any one of claims 36 to 55, wherein the method comprises administering an effective amount of the vector.

57. The method of any one of claims 36 to 56, wherein the disease or disorder is related to or caused by truncation of BAG3 in the subject.

58. The method of any one of claims 36 to 57, wherein the method comprises administering a pharmaceutical composition comprising an effective amount of the vector.

59. The method of any one of claims 36 to 58, wherein the method comprises administering between about 1×10¹¹ vector genomes and about 1×10¹³ vector genomes of the vector to the subject, administering between about 1×10¹² vector genomes and about 1×10¹⁴ vector genomes of the vector to the subject, or administering between about 1×10¹³ vector genomes and about 1×10¹⁵ vector genomes of the vector to the subject.

60. A pharmaceutical composition comprising the vector of any one of claims 26 to 35.

61. A kit comprising the vector of any one of claims 26 to 35 or the pharmaceutical composition of claim 60 and optionally instructions for use.

62. Use of the vector of any one of claims 26 to 35 in treating a disease or disorder, optionally according to the method of any one of claims 39 to 63.

63. A vector according to any one of claims 29 to 38 for use in treating a disease or disorder, optionally according to the method of any one of claims 36 to 59.

64. A polynucleotide, comprising a polynucleotide sequences that shares at least 90%, 91%, 92%, 93%, 94%, 95%, 96%, 97%, 98%, 99%, or 100% identity to any one of SEQ ID NOs: 107-116.

65. The polynucleotide of claim 64, wherein the promoter is a MHCK7 promoter.

66. The polynucleotide of claim 65, wherein the MHCK7 promoter shares at least 75%, 80%, 85%, 90%, 95%, 96%, 97%, 98%, 99% or 100% identity with SEQ ID NO: 31.

67. The polynucleotide of claim 64, wherein the BAG3 is a human BAG3.

68. A gene therapy vector, comprising the polynucleotide of any one of claims 64 to 67.

69. The vector of claim 68, wherein the gene therapy vector is a recombinant adeno-associated virus (rAAV) vector.

70. The vector of claim 69, wherein the rAAV vector is an AArh74 vector.

71. The vector of claim 69, wherein the rAAV vector is an AAV9 vector.

72. A method of treating and/or preventing a cardiac disorder in a subject identified as having a mutation or truncation in BAG3, comprising administering the vector of any one of claims 68 to 71 to the subject.

73. The method of claim 72, wherein the disease or disorder is a cardiomyopathy, optionally a dilated cardiomyopathy (DCM).

74. The method of claim 72, wherein the cardiac disorder is arrhythmia, optionally atrial fibrillation or sinus node disease, or BAG3-related dilated cardiomyopathy.

75. The method of claim 72, wherein the cardiac disorder is heart failure.

76. The method of any one of claims 72 to 75, wherein the subject is a mammal.

77. The method of any one of claims 72 to 76, wherein the vector is administered by intravenous injection, intracardiac injection, intracardiac infusion, and/or cardiac catheterization.
